SEQUENCE LISTING (64 sequences)

SEQ ID NO: 1 - DNA, 381 bp, Homo sapiens
caggtccaactggtacagtctggggctgaggtgaagaagcctggggcctcagtgaaggtctcctgcaaggtttccggatacagcctcactgaattatccatacactgggtgcgacaggctcctggaaaagggcttgaatggatgggaggttttgatcctgaagatggtgaaataatctttgcacagaagttccagggcagagtcaccctgaccgaggacatatctctagacacagcctacatggaggtgagcagcctgagatctgaggacacggccgtgtattactgtgcaacagaggggcatgactccggtgactacgtaggctattactactacggtttggacgtctggggccaagggaccacggtcaccgtctcctca

SEQ ID NO: 2 - protein, 127 aa, Homo Sapiens
QVQLVQSGAEVKKPGASVKVSCKVSGYSLTELSIHWVRQAPGKGLEWMGGFDPEDGEIIFAQKFQGRVTLTEDISLDTAYMEVSSLRSEDTAVYYCATEGHDSGDYVGYYYYGLDVWGQGTTVTVSS

SEQ ID NO: 3 - DNA, 321 bp, Homo Sapiens
gacatccagatgacccagtctccatcctccctgtctgcatctgtaggagacagagtcaccatcacttgccgggcgagtcaggacattagaaattttttagcctggtatcagcagaaaccagggaaacttcctaagctcctgatctatgctgcatccactttgcaatcaggggtcccatctcggttcagtggcagtggatctgggacagatttcactctcaccatcagcagcctgcagcctgaagatgttgcaagttattactgtcaaagccataacagtgtccccttcactttcggccctgggaccaaagtggatatcaga

SEQ ID NO: 4 - protein, 107 aa, Homo Sapiens
DIQMTQSPSSLSASVGDRVTITCRASQDIRNFLAWYQQKPGKLPKLLIYAASTLQSGVPSRFSGSGSGTDFTLTISSLQPEDVASYYCQSHNSVPFTFGPGTKVDIR

SEQ ID NO: 5 - DNA, 369 bp, Homo Sapiens
caggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatcttcgcacagaagttccagggcagagtcaccatgaccgaggacacatctacagacacagcctacatggagctgagcagcctgagatctgaggacacggccgtgtattactgtgcaacgggattacgatttttggagtggttcctatggggatgggacgtctggggccaagggaccacggtcaccgtctcctca

SEQ ID NO: 6 - protein, 123 aa, Homo Sapiens
QVQLVQSGAEVKKPGASVKVSCKVSGYTLTELSMHWVRQAPGKGLEWMGGFDPEDGETIFAQKFQGRVTMTEDTSTDTAYMELSSLRSEDTAVYYCATGLRFLEWFLWGWDVWGQGTTVTVSS

SEQ ID NO: 7 - DNA, 321 bp, Homo Sapiens
gacatccagatgacccagtctccatcttccgtgtctgcatctgtgggagacagagtcaccatcacttgtcgggcgagtcagggtattagcaactggttagcctggtatcagcagacaccggggaaagcccctcagctcctgatctatggtgcatccagtttgcaaagtggggtcccatcaaggttcagcggcagtggatctgggacagatttcactctcaccatcagcagcctgcagcctgaagattttgcaacttactattgtcaacaggctgacagtttccctccgacgttcggccaagggaccaaggtggagatcaaa

SEQ ID NO: 8 - protein, 107 aa, Homo Sapiens
DIQMTQSPSSVSASVGDRVTITCRASQGISNWLAWYQQTPGKAPQLLIYGASSLQSGVPSRFSGSGSGTDFTLTISSLQPEDFATYYCQQADSFPPTFGQGTKVEIK

SEQ ID NO: 9 - DNA, 381 bp, Homo Sapiens
caggtccagctggtacagtccggggctgaggtgaagaagcctggggcctcagtgaaggtctcctgcaaggtttccggagacaccctcactgaattatccatgcactgggtgcgacagactcctggaaaagggcttgagtggatgggaggttttgatcctgaagatggtgaaacaatctacgcacagaagttccagggcagagtcaccatgaccggggacacatctacagacacagcctacatggagctgagcagcctgagatctgaggacacggccgtgtattactgtgcaacagagggggactacggtgattcagaggagtactactactactacggtatggacgtctggggccaagggaccacggtcaccgtctcctca

SEQ ID NO: 10 - protein, 127 aa, Homo Sapiens
QVQLVQSGAEVKKPGASVKVSCKVSGDTLTELSMHWVRQTPGKGLEWMGGFDPEDGETIYAQKFQGRVTMTGDTSTDTAYMELSSLRSEDTAVYYCATEGDYGDSEEYYYYYGMDVWGQGTTVTVSS

SEQ ID NO: 11 - DNA, 321 bp, Homo Sapiens
gacatccagatgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataaccgtgccccattcactttcggccctgggaccaaagtggatatcaaa

SEQ ID NO: 12 - protein, 107 aa, Homo Sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNRAPFTFGPGTKVDIK

SEQ ID NO: 13 - DNA, 369 bp, Homo Sapiens
caggtgcagctggtggagtctgggggaggcgtggtccagcctgggaggtccctgagactctcctgtgcagcgtctggattcaccttcagtagttatggcatgcactgggtccgccaggctccaggcaaggggctggagtgggtggcagttatatggtatgatggaagtaataaatactatgcagactccgtgaagggccgattcaccatctccagagacaattccaagaacacgctgaatctgcaaatgaacagcctgagagccgaggacacggctgtgtattactgtgggagagatcctgggacctaccactactactatcacggtatggacgtctggggccaagggaccacggtcaccgtctcctca

SEQ ID NO: 14 - protein, 123 aa, Homo Sapiens
QVQLVESGGGVVQPGRSLRLSCAASGFTFSSYGMHWVRQAPGKGLEWVAVIWYDGSNKYYADSVKGRFTISRDNSKNTLNLQMNSLRAEDTAVYYCGRDPGTYHYYYHGMDVWGQGTTVTVSS

SEQ ID NO: 15 - DNA, 336 bp, Homo Sapiens
tatattgtgatgactcagtctccactctccctgcccgtcacccctggagagccggcctccatctcctgcaggtctagtcagagcctcctgcatagtaatggatacaactttttggattggtacctgcagaagccagggcagtctccacagctcctgatctatttgggttctaatcgggcctccggggtccctgacaggttcagtggcagtggatcaggcacagattttacactgaaaatcaacagagtggaggctgaggatgttggggtttattactgcatgcaagctctacaaactcccatcaccttcggccaagggacacgactggagattaaa

SEQ ID NO: 16 - protein, 112 aa, Homo Sapiens
YIVMTQSPLSLPVTPGEPASISCRSSQSLLHSNGYNFLDWYLQKPGQSPQLLIYLGSNRASGVPDRFSGSGSGTDFTLKINRVEAEDVGVYYCMQALQTPITFGQGTRLEIK

SEQ ID NO: 17 - DNA, 369 bp, Homo Sapiens; identical to SEQ ID NO: 13

SEQ ID NO: 18 - protein, 123 aa, Homo Sapiens; identical to SEQ ID NO: 14

SEQ ID NO: 19 - DNA, 321 bp, Homo Sapiens
caaatagtgatgacgcagtctccagccaccttgtctgtgtctccaggggaaagagccaccctctcctgcagggccagtcagagtgttagcagaaacttagcctggtaccagcagaaacctggccagtctcccaggctccttatctatggtgcatccaccagggccactggtttcccagccaggatcagtggcagtgggtctgggacagagttcactctcaccatcagcagccttcagtctgaagattttgcagtttattactgtcagcagtataatcactggcctcagactttcggcggagggaccgaggtggagatcaaa

SEQ ID NO: 20 - protein, 107 aa, Homo Sapiens
QIVMTQSPATLSVSPGERATLSCRASQSVSRNLAWYQQKPGQSPRLLIYGASTRATGFPARISGSGSGTEFTLTISSLQSEDFAVYYCQQYNHWPQTFGGGTEVEIK

SEQ ID NO: 21 - DNA, 366 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagaaggggccccgtatagcagctccgatgcttttgatatctggggccaagggacaatggtcaccgtctcttca

SEQ ID NO: 22 - protein, 122 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREGAPYSSSDAFDIWGQGTMVTVSS

SEQ ID NO: 23 - DNA, 333 bp, Homo Sapiens
cagtctgtgctgacgcagccgccctcagtgtctggggccccagggcagagggtcaccatctcctgcactgggagcagctccaacatcggggcaaattatgatgtacactggtaccagcagcttccaggaacagcccccaaactcctcatctatggtgacaacaatcggccctcaggggtccctgaccgattctctggctccaagtctggcacctcagcctccctggccatcactgggctccaggctgaggatgaggctgattattactgccagtcctatgacagcagcctgagtggttcggtattcggcggagggaccaagctgaccgtccta

SEQ ID NO: 24 - protein, 111 aa, Homo Sapiens
QSVLTQPPSVSGAPGQRVTISCTGSSSNIGANYDVHWYQQLPGTAPKLLIYGDNNRPSGVPDRFSGSKSGTSASLAITGLQAEDEADYYCQSYDSSLSGSVFGGGTKLTVL

SEQ ID NO: 25 - DNA, 369 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaagactatgcagactccgtgaagggccgattcaccatctccagagacaattccaagaacacgctgtatctgcaaatgaacagcctgagagccgaggacacggctgtgtattactgtgcgagagagggtagcaccagctggacggactactacggtatggacgtctggggccaagggaccacggtcaccgtctcctca

SEQ ID NO: 26 - protein, 123 aa, Homo Sapiens
QVQLVESGGGVVQPGRSLRLSCAASGFTFSSYGMHWVRQAPGKGLEWVAVIWYDGSNKDYADSVKGRFTISRDNSKNTLYLQMNSLRAEDTAVYYCAREGSTSWTDYYGMDVWGQGTTVTVSS

SEQ ID NO: 27 - DNA, 324 bp, Homo Sapiens
tcctatgagctgacacagccaccctcggtgtcagtgtccccaggacaaacggccaggatcacctgctctggagatgcattgccaaaaagatatgcttattggtaccagcagaagtcaggccaggcccttgtgctggtcatctatgaggacagcaaacgaccctccgggatccctgagagattctctggctccagctcagggacaatggccaccttgactatcagtggggcccaggtggaggatgaagctgactactactgttactcaatagacagcagtggtaatcatggggtgttcggcggagggaccaagctgaccgtccta

SEQ ID NO: 28 - protein, 108 aa, Homo Sapiens
SYELTQPPSVSVSPGQTARITCSGDALPKRYAYWYQQKSGQALVLVIYEDSKRPSGIPERFSGSSSGTMATLTISGAQVEDEADYYCYSIDSSGNHGVFGGGTKLTVL

SEQ ID NO: 29 - DNA, 366 bp, Homo Sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggttcatctattacagtgggagcaccttctacaacccgtccctcaagagtcgaattaccatatcagttgacacgtctaagaaccagttctccctgaagctgagttctgtgactgccgcggacacggccgtgtattactgtgcgagaccgtatagcagcagctggtacggtgcttttgatatctggggccaagggacaatggtcaccgtctcttca

SEQ ID NO: 30 - protein, 122 aa, Homo Sapiens
QVQLQESGPGLVKPSQTLSLTCTVSGGSISSGGYYWSWIRQHPGKGLEWIGFIYYSGSTFYNPSLKSRITISVDTSKNQFSLKLSSVTAADTAVYYCARPYSSSWYGAFDIWGQGTMVTVSS

SEQ ID NO: 31 - DNA, 318 bp, Homo Sapiens
tcctatgagctgactcagccaccctcagtgtccgtgtccccaggacggacagccagcatcacctgctctgaagataaattggggaataaatatgcttactggtatcagcagaagccaggccagtcccctgtgctggtcatctatcaagatagcgagcggccctcaggaatccctgagcgattctctggctccaactctgggaacacagccactctgaccatcagcgggacccaggctatggatgaggctgactattactgtcaggcgtgggacagcagcactgtggtattcggcggagggaccaagctgaccgtccta

SEQ ID NO: 32 - protein, 106 aa, Homo Sapiens
SYELTQPPSVSVSPGRTASITCSEDKLGNKYAYWYQQKPGQSPVLVIYQDSERPSGIPERFSGSNSGNTATLTISGTQAMDEADYYCQAWDSSTVVFGGGTKLTVL

SEQ ID NO: 33 - protein, 123 aa, Homo Sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDYGDYYYYYYGMDVWGQGTTVTVSS

SEQ ID NO: 34 - protein, 122 aa, Homo Sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDYGDYYYYYGMDVWGQGTTVTVSS

SEQ ID NO: 35 - protein, 123 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDYGDYYYYYYGMDVWGQGTTVTVSS

SEQ ID NO: 36 - protein, 121 aa, Homo Sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATLRFLEWLLGMDVWGQGTTVTVSS

SEQ ID NO: 37 - protein, 117 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYSSSAFDIWGQGTMVTVSS

SEQ ID NO: 38 - protein, 117 aa, Homo Sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARYSSAFDIWGQGTMVTVSS

SEQ ID NO: 39 - protein, 119 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARSSSWYYGMDVWGQGTTVTVSS

SEQ ID NO: 40 - protein, 107 aa, Homo Sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPFTFGPGTKVDIK

SEQ ID NO: 41 - protein, 112 aa, Homo Sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGQGTRLEIK

SEQ ID NO: 42 - protein, 107 aa, Homo Sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 43 - protein, 107 aa, Homo Sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPWTFGQGTKVEIK

SEQ ID NO: 44 - protein, 111 aa, Homo Sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGVVFGGGTKLTVL

SEQ ID NO: 45 - protein, 107 aa, Homo Sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTAVVFGGGTKLTVL

SEQ ID NO: 46 - protein, 108 aa, Homo Sapiens
SYELTQPPSVSVSPGQTARITCSGDALPKKYAYWYQQKSGQAPVLVIYEDSKRPSGIPERFSGSSSGTMATLTISGAQVEDEADYYCYSTDSSGNHVVFGGGTKLTVL

SEQ ID NO: 47 - protein, 5 aa, Homo sapiens
ELSIH

SEQ ID NO: 48 - protein, 17 aa, Homo sapiens
GFDPEDGEIIFAQKFQG

SEQ ID NO: 49 - protein, 18 aa, Homo sapiens
EGHDSGDYVGYYYYGLDV

SEQ ID NO: 50 - protein, 5 aa, Homo sapiens
ELSMH

SEQ ID NO: 51 - protein, 17 aa, Homo sapiens
GFDPEDGETIYAQKFQG

SEQ ID NO: 52 - protein, 18 aa, Homo sapiens
EGDYGDSEEYYYYYGMDV

SEQ ID NO: 53 - protein, 5 aa, Homo sapiens
ELSMH

SEQ ID NO: 54 - protein, 17 aa, Homo sapiens
GFDPEDGETIFAQKFQG

SEQ ID NO: 55 - protein, 14 aa, Homo sapiens
GLRFLEWFLWGWDV

SEQ ID NO: 56 - protein, 11 aa, Homo sapiens
RASQDIRNFLA

SEQ ID NO: 57 - protein, 7 aa, Homo sapiens
AASTLQS

SEQ ID NO: 58 - protein, 9 aa, Homo sapiens
QSHNSVPFT

SEQ ID NO: 59 - protein, 11 aa, Homo sapiens
RASQGISNYLA

SEQ ID NO: 60 - protein, 7 aa, Homo sapiens
AASTLQS

SEQ ID NO: 61 - protein, 9 aa, Homo sapiens
QKYNRAPFT

SEQ ID NO: 62 - protein, 11 aa, Homo sapiens
RASQGISNWLA

SEQ ID NO: 63 - protein, 7 aa, Homo sapiens
GASSLQS

SEQ ID NO: 64 - protein, 9 aa, Homo sapiens
QQADSFPPT

(19) United States
(12) Patent Application Publication
(10) Pub. No.: US 2010/0233154 A1
(43) Pub. Date: Sep. 16, 2010

(54) TARGETED BINDING AGENTS DIRECTED TO HEPARANASE AND USES THEREOF 463

(75) Inventors: Paul Elvin, Cheshire (GB); Chadwick King, North Vancouver (CA); Palaniswami Rathanaswami, Burnaby (CA)

Correspondence Address: Medimmune, Limited, One Medimmune Way, Gaithersburg, MD, 20878, US

(73) Assignee: MedImmune Limited, Cambridgeshire (GB)

(21) Appl. No.: 12/575,267

(22) Filed: Oct. 7, 2009

Related U.S. Application Data
(60) Provisional application No. 61/103,645, filed on Oct. 8, 2008.

(51) Int. Cl. A61K 39/395; C07K 16/40; C07K 16/00; C07H 21/04; A61P 35/00; A61P 9/10; A61P 19/02; C12N 15/63; C12N 5/07

(52) U.S. Cl. 424/130.1; 530/387.1; 530/388.26; 536/23.53; 530/388.15; 435/320.1; 435/325

(57) ABSTRACT

The invention relates to targeted binding agents that specifically bind to heparanase and inhibit the biological activity of heparanase and uses of such agents. More specifically the invention relates to fully human monoclonal antibodies directed to that specifically bind to heparanase and uses of these antibodies. Aspects of the invention also relate to hybridomas or other cell lines expressing such antibodies. The disclosed targeted binding agents and antibodies are useful as diagnostics and for the treatment of diseases associated with the activity and/or overexpression of heparanase.

This application claims the benefit under 35 U.S.C. §119(e) of U.S. Provisional Application Ser. No. 61/103,645 filed on Oct. 8, 2008, the entirety of which is hereby incorporated by reference.

### FIELD OF THE INVENTION

The invention relates to targeted binding agents against the target antigen heparanase 1 (Hpa1, referred to hereinafter as heparanase) and uses of such agents. In some embodiments, the invention relates to fully human monoclonal antibodies directed to heparanase and uses of these antibodies. Aspects of the invention also relate to hybridomas or other cell lines expressing such targeted binding agents or antibodies. The described targeted binding agents are useful as diagnostics and for the treatment of diseases associated with the activity and/or overproduction of heparanase.

### DESCRIPTION OF THE RELATED ART

Heparan sulfate proteoglycans (HSPGs) are widely distributed in tissues and have important regulatory and structural functions in the extracellular matrix and at the cell surface (for review see Bishop et al, Nature 446 1030-1037 2007). In the extracellular matrix (ECM) the major family of HSPG, perlecans, contribute to ECM structure through binding interactions with proteins such as fibronectin, laminin and collagens (Farach-Carson and Carson, Glycobiol 17 897-905 2007). In addition, ECM HSPG provide sites of sequestration for a number of heparin binding growth factors with important roles in tissue remodelling and cancer, including HGF, HB-EGF, FGF, TGFβ and VEGF. Cell surface HSPGs function as co-receptors for ligands such as FGF, aiding correct presentation to the cognate growth factor receptors. Two families of cell surface HSPG, the syndecans and glypicans, function as regulators of cell growth through interactions with cell adhesion receptors and other signalling pathways. Both syndecans (Fears et al, Matrix Biol 25 443-456 2006) and glypicans (Filmus, Glycobiol 11 19-23 2001) have also been implicated in the regulation of tumour growth. Modification of HSPG in the tumour microenvironment by sulfatase and heparanase activity, resulting in the removal of O-sulfation or fragmentation of heparan sulfate (HS) respectively, can thus have far reaching effects on tumour cell behaviour, tumour growth and progression (Sanderson et al, J. Cellular Biochem 96 897-905 2005).

Although a number of proteins were originally described as having heparanase-like activity (Kosir et al, J Surg Res 103 100-108 2002; Hoogewerf et al, J Biol Chem 270 3268-3277 1995), later experiments to characterise the activity identified a contaminating enzyme in cellular lysates (Castor et al J Rheumatol 29 2337-2344 2002) which led to the identification of mammalian heparanase (Kussie et al, Biochem Biophys Res Comm 261 183-187 1999). The cloning of human heparanase (Vlodaysky et al, Nature Med 5 793-802 1999; Hulet et al, Nature Med 5 803-809 1999) confirmed a single heparanase gene and a role for heparanase in tumour metastasis. The role of heparanase in disease progression in cancer and other pathologies has been the subject of many reviews (e.g. Vlodaysky et al, Curr Pharm Design 13 2057-2073 2007; McKenzie, Br J Pharmacol 151 1-14 2007).

Human heparanase is a 543 amino acid endoglycosidase that cleaves the glycosidic bonds within low sulfation domains found in HSPG to yield characteristic 5-7 kDa fragments. Heparanase is secreted via the ER/golgi pathway as an inactive 65 kDa protein that is efficiently taken up into the cell. The low-density lipoprotein receptor (LRP), mannose-6-phosphate receptor (M6R) (Vreys et al, J Biol Chem 280 33141 2005) and cell surface HS (Gingis-Velitski et al, J Biol Chem 279 44084 2004) have all been implicated as the cellular uptake partner. However, in vitro experiments using cell lines deficient in each of the aforementioned receptors suggests that an alternative mechanism may account for uptake, and an unidentified component of cell surface lipid rafts was suggested to be responsible for heparanase uptake (Ben-Zaken et al, Biochem Biophys Res Comm 361 829-834 2007).

Once in the lysosomal compartment, the 65 kDa form of heparanase is converted to the active heterodimeric form (Zester et al, J Cell Science 117 2249 2003) by proteolytic cleavage at two sites, to generate 8 and 50 kDa subunits that comprise the active heterodimeric form of the enzyme (Abboud-Jarrous et al J Biol Chem 280 13568 2005; Levy-Adam et al, Biochem Biophys Res Comm 308 885-891 2003), most likely through the activity of cathepsin L (Abboud-Jarrous et al, J Biol Chem 283 18167 2008). Active site residues, Glu225 and Glu343, and heparin binding domains reside within the 50 kDa subunit (Levy-Adam et al., J Biol Chem 280 20457-20466 2005), and the 8 kDa sub-unit is required to confer activity. Immunohistochemical and immunofluorescent localisation of heparanase in tissues and cells has shown a perinuclear distribution in lysosomal bodies where the enzyme is stable for at least 30 hrs (Gingis-Velitski et al, J Biol Chem 279 44084 2004) and degradation of internalised HSPG takes place within the endosome/lysosome compartment. Cell fractionation experiments have also identified a significant fraction of heparanase within the nucleus (Schubert et al, Lab Invest 84 535 2004) where heparanase modification of nuclear HS may regulate gene expression and differentiation (Nobuhisa et al, Cancer Sci 98 535 2007).

Degradation and modification of ECM or cell surface HSPG requires a secretory route for heparanase and activity outside of the lysosomal compartment. Although the secretory route for active heparanase to the cell surface has not been fully defined, a PKA/PKC dependent pathway has been suggested, based on the observation that activation of PY2 receptors in response to nucleoside stimulation was shown to lead to a rapid PKC dependent increase in cell surface heparanase activity (Shafat et al, J Biol Chem 281 23804-23811 2006). Alternatively, secretion of heparanase may be a result of Rab/ARF regulated endosomal recycling (Jones et al, Curr Opinion Cell Biol 18 549-557 2006), although there are no data to support this hypothesis. Once in the extracellular space, efficient uptake of the enzyme may serve to regulate activity against cell surface and ECM HSPG.

Cell surface localisation of heparanase has been demonstrated in tumour cells (Goldshmidt et al, PNAS 99 10031 2002) and macrophages (Sasaki et al, J Immunol 172 3830 2004), and heparanase is released by activated platelets (J Cellular Physiol 175 255 1998) and endothelial cells (Chen et al, Biochemistry 43 4971 2004), observations that are consistent with extracellular heparanase activity and the proposed role in tumour cell invasion and metastasis (Hulett et al, Nature Med 5 803 1999; Edovitsky et al, J Nat Cancer Inst 96 1219 2004). Heparanase activity has also been implicated in the regulation of angiogenesis (Elkin et al, FASEB J 15 1661-1663 2001), restenosis (Francis et al, Circulation Res 92 e70-e77 2003), hair growth (Zcharia et al, Am J Pathol 166 999-1008 2005), implantation (Revel et al, Fertility and Sterility 83 580-586 2004), tissue morphogenesis and kidney function (Zcharia et al, FASEB J 18 252-263 2004; van den Hoven et al, Kidney Int 70 2100-2108 2006; Holt et al, Kidney Int. 67 122-129 2005), bone formation (Kram et al, J Cell Physiol 207 784-792 2006) and inflammatory bowel disease (Waterman et al, Modern Pathol 20 8-14 2007).

Upregulation of heparanase activity may influence tumour growth and progression through mechanisms that reflect direct modification of extracellular matrix HSPG and thus facilitating local tumour extension and metastasis (Edovitsky et al, J Nat Cancer Inst 96 1219-1230 2004; Zhang et al, Biochem Biophys Res Comm 358 124-129 2007), and an indirect effect on cell proliferation as a consequence of the release of heparin binding growth factors such as bFGF (Ishai-Michaeli et al, Cell Reg 1 833-842 1990), HB-EGF, VEGF, TGFβ, and HGF from sites of HSPG sequestration. Modification of cell surface HSPG by heparanase may also regulate both the uptake and presentation of heparin binding growth factors to their cognate receptors, as has been demonstrated for bFGF (Roghani and Moscatelli, J Biol Chem 267 22156 1992; Richardson et al, J Biol Chem 274 13534 1999). Heparanase may also modify the response of tumour cells to regulation by cell surface HSPG, and has been implicated in the shedding of syndecan-1 (J Biol Chem 282 13326-13333 2007) that has a stimulatory effect on tumour cell proliferation. Independent of enzymatic activity, heparanase may also contribute to the repertoire of cell adhesion molecules, since a cell surface localised form of heparanase increased the adhesion of non-adhesive murine Eb lymphoma cells (Goldschmidt et al, FASEB J 17 1015-1025 2003). Heparanase has also been shown to have a direct effect on the expression of VEGF, phosphorylation of Akt and p38 (Zester at al, Cancer Res 66 1455-1463 2006; Ben-Zaken et al, Biochem Biophys Res Comm 361 829-834 2007), all of which contribute to tumour progression.

Elevated expression of heparanase has been associated with disease progression and poor survival in a number of human cancers including breast (Maxheimer et al., Surgery 132 326-333 2002; Maxheimer et al, J Am Coll Surg 200 328-335 2005), colon (Nobuhisa et al, J Cancer Res Clin Oncol 131 229-237 2005), head and neck (Doweck et al, Neoplasia 8 1055-1061 2006), pancreatic (Schoppmeyer et al, Pancreatology 5 570-575 2005), ovarian (Davidson et al, Gynaecol Oncol 104 311-319 2007), NSCLC (Takahashi et al, Lung Cancer 45 207-214 2004), bladder (Gohji et al, J Urol 166 1286-1290 2001), and gastric cancers (Tang et al, Mod Pathol 15 593-598 2002). Generally, heparanase expression, as measured by both mRNA and immunohistochemical methods, is correlated with increasing tumour size, stage of disease and metastatic spread.

The clinical evidence associating heparanase expression and activity with tumour progression is supported by mechanistic studies in vitro and in vivo. Antisense heparanase, although having no effect on in vitro proliferation of A549 lung cancer cells, inhibited the growth of tumours in vivo (Uno et al, Cancer Res 61 7855-7860 2001) and similarly inhibited the growth of hepatocellular tumours (Zhang et al, Biochem Biophys Res Comm 358 124-129 2007). In contrast, overexpression of heparanase in MCF-7 breast tumour cells increased tumour growth and vascularity in vivo (Cohen et al, Int J Cancer 118 1609-1617 2006), and increased expression of heparanase in myeloma cells resulted in increased primary tumour growth and metastasis to bone (Yang et al, Blood 105 1303-1309 2005). The response of tumour cells to increased heparanase expression/activity may depend on the cellular background and context. High levels of heparanase expression were growth inhibitory while relatively modest increases in heparanase expression promoted the growth of U87 glioma in vivo (Zester et al, Cancer Res 63 7733-7741 2003).

The importance of heparanase to tumour progression and angiogenesis has led to the search for pharmacologically useful inhibitors of heparanase activity. Substrate mimetics such as PI-88 and analogues (Karoli et al, J Med Chem 48 8229-8236 2005); oligomannurarate JG3 (Zhao et al, Cancer Res 66 8779-8787 2006); and small molecule inhibitors (Ishida et al, Mol Cancer Therap 3 1069-1077 2004; J Org Chem 70 8884-8889 2005; Xu et al, Bioorg Med Chem Lett 16 404-408 2006; Pan et al, Bioorg Med Chem Lett 16 409-412 2006) have demonstrated selective inhibition of heparanase activity and heparanase mediated responses such as cell invasion, proliferation and angiogenesis. PI-88 is the most clinically advanced heparanase inhibitor having successfully completed a Phase II trial in hepatocellular cancer. However, PI-88 has a complex mode of action inhibiting both heparanase activity and the binding of growth factors to HS whereas other small molecule inhibitors target the enzyme activity alone.

In addition to small molecular weight inhibitors and molecular targeting approaches, anti-heparanase antibodies that inhibit heparanase activity and subsequent cellular responses have been described. For example, a heparanase neutralising rabbit polyclonal antibody inhibited both enzyme activity and in vitro invasion through matrigel (He et al, Cancer Res 64 3928-3933 2004) in an ovarian tumour cell line. In a second example a heparanase neutralising antibody, raised in rabbits to a peptide spanning the active site of heparanase, was shown to inhibit neointimal thickening in a balloon catheter arterial injury model in vivo (Myler et al, J Biochem 139 339-345 2006). An antibody raised in rabbits to a peptide sequence from the amino terminus of the 50 kDa subunit of heparanase was also shown to inhibit heparanase activity in vitro (Zester et al, J Cell Science 117 2248 2003), suggesting that in addition to interactions between the large and small heparanase sub-units, a number of epitopes contribute to the active conformation of heparanase. Rabbit polyclonal antibodies binding to human heparanase were also described in WO99/43830 (Pharmacia & Upjohn) although no associated activities were reported. Anti-heparanase IgM monoclonal antibody 10E5 (ATCC-HB11403) was raised against murine melanoma heparanase and has been shown to recognize human heparanase (Mollinedo et al (1997) Biochem J. 327, 917-923). Murine monoclonal antibodies raised against recombinant human heparanase, having neutralising activity in a heparan sulfate degradation assay were described by Insight in WO00/25817. Murine antibody HP-130 disclosed in WO00/25817 which cross reacts with chicken heparanase and with the 65 kDa precursor of human heparanase (pro heparanase), inhibited the activity of recombinant human heparanase, or purified human placental heparanase, at a molar ratio of approximately 1:10 or 1:20 enzyme:antibody respectively. More recently, Gingis-Velitski et al (FASEB J 21 3986-3993 2007) in an attempt to isolate antibodies with heparanase inhibitory activity, described the isolation of a murine antibody (6F8) that stimulated the activity of heparanase. The antibody, 6F8, stimulated cell invasion in vitro and wound healing in vivo, further supporting the evidence that heparanase plays a key role in regulating biological responses that involve modification of HSPG and the activity of heparin-binding growth factors. In addition to murine antibodies, CellTech (WO2004043989) have described the isolation of transgenic human antibodies from KM mice to heparanase that inhibited heparanase activity (antibody 22D9 reported therein to have an affinity of K_{D} 2.5 nM).

### SUMMARY OF THE INVENTION

The present invention relates to targeted binding agents that specifically bind to heparanase and inhibit the biological activity of heparanase. Further embodiments of the invention relate to targeted binding agents that specifically bind to heparanase and thereby inhibit heparanase enzyme activity. Further embodiments of the invention relate to targeted binding agents that specifically bind to heparanase and thereby inhibit the degradation of heparan sulfate proteoglycans (HSPGs). Further embodiments of the invention relate to targeted binding agents that specifically bind to heparanase and thereby inhibit phosphorylation of ERK1/2. Further embodiments of the invention relate to targeted binding agents that specifically bind to heparanase and thereby inhibit cell proliferation (for example tumour or endothelial cell proliferation). Further embodiments of the invention relate to targeted binding agents that specifically bind to heparanase and thereby inhibit the release of bFGF from heparan sulfate. Further embodiments of the invention relate to targeted binding agents that specifically bind to heparanase and inhibit heparanase enzyme activity, and also bind to and inhibit the heparanase enzyme activity of heparanase from other species, including, but not limited to murine and cynomolgus heparanase.

Further embodiments of the invention relate to targeted binding agents that specifically bind to heparanase and thereby inhibit the binding of heparanase to its substrates.

Further embodiments of the invention relate to targeted binding agents that specifically bind to pro-heparanase and thereby inhibit the activation of pro-heparanase to heparanase.

Further embodiments of the invention relate to targeted binding agents that specifically bind to pro-heparanase and thereby inhibit the internalisation and activation of pro-heparanase to a fully active form.

Further embodiments of the invention relate to targeted binding agents that specifically bind to heparanase and thereby reduce the availability of heparin binding growth factors by preventing their dissociation from the ECM.

Further embodiments of the invention relate to targeted binding agents that specifically bind to heparanase and thereby inhibit the adhesion of cells to the extracellular matrix (ECM).

Further embodiments of the invention relate to targeted binding agents that specifically bind to heparanase and thereby inhibit the modification or shedding of cell surface HSPGs.

Further embodiments of the invention relate to targeted binding agents that specifically bind to heparanase and thereby inhibit the degradation of HSPGs in the ECM thus preventing tumour cell invasion.

Further embodiments of the invention relate to targeted binding agents that specifically bind to heparanase and thereby inhibit established tumour growth (tumours greater than or equal to 0.2 cm³).

Embodiments of the invention relate to targeted binding agents that specifically bind to heparanase and inhibit the biological activity of heparanase. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the biological activity that would occur in the absence of the targeted binding agent.

Embodiments of the invention relate to targeted binding agents that specifically bind to heparanase and thereby inhibit heparanase enzyme activity. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of heparanase enzyme activity that would occur in the absence of the targeted binding agent.

Embodiments of the invention relate to targeted binding agents that specifically bind to heparanase and thereby inhibit human heparanase enzyme activity. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of human heparanase enzyme activity that would occur in the absence of the targeted binding agent.

Further embodiments of the invention relate to targeted binding agents that specifically bind to heparanase and inhibit heparanase enzyme activity, and also bind to and inhibit heparanase enzyme activity from other species, including, but not limited to murine and cynomolgus heparanase. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the mouse and/or cynomolgus heparanase enzyme activity that would occur in the absence of the targeted binding agent.

Embodiments of the invention relate to targeted binding agents that specifically bind to heparanase and thereby inhibit >90% of heparanase activity at a molar ratio of approximately 1:30 of enzyme:antibody. Further embodiments of the invention relate to targeted binding agents that specifically bind to heparanase and inhibit >90% of heparanase activity that would occur in the absence of the targeted binding agent at a molar ratio of approximately 1:30 of enzyme:antibody. Further embodiments of the invention relate to targeted binding agents that specifically bind to heparanase and inhibit >90% of heparanase activity that would occur in the absence of the targeted binding agent at a molar ratio of approximately 1:20 or 1:10 of enzyme:antibody.

Further embodiments of the invention relate to targeted binding agents that specifically bind to heparanase and thereby inhibit the degradation of heparan sulfate proteoglycans. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the degradation of HSPGs that would occur in the absence of the targeted binding agent.

Further embodiments of the invention relate to targeted binding agents that specifically bind to heparanase and thereby inhibit the degradation of HSPGs in the ECM. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the degradation of HSPGs in the ECM that would occur in the absence of the targeted binding agent.

Further embodiments of the invention relate to targeted binding agents that specifically bind to heparanase and thereby inhibit phosphorylation of ERK1/2. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the phosphorylation of ERK1/2 that would occur in the absence of the targeted binding agent.

Further embodiments of the invention relate to targeted binding agents that specifically bind to heparanase and thereby inhibit cell proliferation. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of cell proliferation that would occur in the absence of the targeted binding agent.

Further embodiments of the invention relate to targeted binding agents that specifically bind to heparanase and thereby inhibit the release of bFGF from heparan sulfate. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the release of bFGF from heparan sulfate that would occur in the absence of the targeted binding agent.

Further embodiments of the invention relate to targeted binding agents that specifically bind to heparanase and thereby inhibit the binding of heparanase to its substrates. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the binding of heparanase to its substrates that would occur in the absence of the targeted binding agent.

Further embodiments of the invention relate to targeted binding agents that specifically bind to pro-heparanase and thereby inhibit the internalisation and activation of pro-heparanase to a fully active form. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the internalisation and activation of pro-heparanase to a fully active form that would occur in the absence of the targeted binding agent.

Further embodiments of the invention relate to targeted binding agents that specifically bind to pro-heparanase and thereby inhibit the internalisation and activation of pro-heparanase to heparanase. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the internalisation and activation of pro-heparanase to heparanase that would occur in the absence of the targeted binding agent.

Further embodiments of the invention relate to targeted binding agents that specifically bind to heparanase and thereby reduce the availability of heparin binding growth factors by preventing their dissociation from the ECM. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of availability of heparin binding growth factors that would occur in the absence of the targeted binding agent.

Further embodiments of the invention relate to targeted binding agents that specifically bind to heparanase and thereby inhibit the adhesion of cells to the ECM. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the adhesion of cells to the ECM that would occur in the absence of the targeted binding agent.

Further embodiments of the invention relate to targeted binding agents that specifically bind to heparanase and thereby inhibit the modification or shedding of cell surface HSPG. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the modification or shedding of cell surface HSPG that would occur in the absence of the targeted binding agent.

Further embodiments of the invention relate to targeted binding agents that specifically bind to heparanase and thereby inhibit the degradation of HSPGs in the ECM thus preventing tumour cell invasion. In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the degradation of HSPGs in the ECM that would occur in the absence of the targeted binding agent.

Further embodiments of the invention relate to targeted binding agents that specifically bind to heparanase and thereby inhibit established tumour growth (tumours greater than or equal to 0.2 cm³). In one embodiment the targeted binding agent inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of established tumour growth that would occur in the absence of the targeted binding agent.

The targeted binding agents also inhibit tumour cell adhesion, motility, invasion, and cellular metastasis and in addition, the targeted binding agents are useful for reducing tumour growth and angiogenesis. Mechanisms by which this can be achieved can include, and are not limited to, inhibiting heparanase activity, inhibiting heparanase enzyme activity, inhibiting the binding of heparanase to its substrates, inhibiting the internalisation and activation of pro-heparanase to a fully active form, inhibiting efficient uptake of the pro-heparanase into the cell and thereby inhibiting subsequent processing and secretion of the active form of heparanase, reducing the availability of heparin binding growth factors by preventing their dissociation from the ECM, inhibiting the adhesion of cells to the ECM, inhibiting the phosphorylation of ERK1/2, inhibiting the release of bFGF from heparan sulfate, inhibiting the release of other heparin-binding growth factors from heparan sulfate, inhibiting the modification or shedding of cell surface HSPG, modification of gene expression and inhibiting the degradation of HSPGs in the ECM thus preventing tumour cell invasion.

In one embodiment of the invention, the targeted binding agent is an antibody. In one embodiment of the invention, the targeted binding agent is a monoclonal antibody. In one embodiment of the invention, the targeted binding agent is a fully human monoclonal antibody. Such monoclonal antibodies may be referred to herein as anti-heparanase antibodies or antibodies of the invention.

Inhibition of the biological activity of heparanase may lead to a modification of cell surface heparan sulfate molecules that serve as co-receptors for certain heparin binding growth factors such as bFGF, HGF. Inhibition of the biological activity of heparanase may inhibit cell proliferation or other responses arising as a consequence of receptor tyrosine kinase activity from receptors such as FGFR, c-met. Inhibition of the biological activity of heparanase may similarly inhibit the release of heparin binding growth factors from the ECM as a result of inhibiting the activity of heparanase. Inhibition of the biological activity of heparanase may prevent the shedding of cell surface molecules such as syndecans, thereby having an inhibitory effect on biological responses mediated by cell surface or shed syndecan fragments. Without wishing to be bound by any particular theoretical considerations, mechanisms by which inhibition of the biological activity of heparanase may impact on tumour progression can arise, but are not limited to, direct modification of heparan sulfate, HSPGs, the release of heparin binding growth factors from ECM or cell surface heparan sulfate binding sites, the modification of cell surface co-receptor activity, and modification or degradation of cell surface HSPGs that have a signalling role and determine cellular proliferative, adhesive and migratory phenotypes.

Antibodies, monoclonal antibodies and human monoclonal antibodies include the antibodies of the IgG1, IgG2, IgG3 and IgG4 isotypes, for example IgG2. In one embodiment of the invention, the targeted binding agent is a fully human monoclonal antibody of the IgG2 isotype. This isotype has reduced potential to elicit effector function in comparison with other isotypes, which may lead to reduced toxicity. In another embodiment of the invention, the targeted binding agent is a fully human monoclonal antibody of the IgG1 isotype. The IgG1 isotype has increased potential to elicit ADCC in comparison with other isotypes, which may lead to improved efficacy. The IgG1 isotype has improved stability in comparison with other isotypes, e.g. IgG4, which may lead to improved bioavailability/ease of manufacture/longer half-life. In one embodiment, the fully human monoclonal antibody of the IgG1 isotype is of the z, za or f allotype. In one embodiment of the invention, the targeted binding agent has desirable therapeutic properties, selected from one or more of high binding affinity for heparanase, the ability to inhibit heparanase activity in vitro and in vivo, and the ability to inhibit heparanase-induced cell adhesion, proliferation, motility, invasion, metastasis, tumour growth and angiogenesis.

In one embodiment, the invention includes antibodies that specifically bind to heparanase with very high affinities (Kd). In some embodiments of the invention, the targeted binding agent binds heparanase with a binding affinity (Kd) of less than 5 nanomolar (nM). In other embodiments, the targeted binding agent binds with a Kd of less than 4 nM, 3 nM, 2.5 nM, 2 nM or 1 nM. In some embodiments of the invention, the targeted binding agent binds heparanase with a Kd of less than 950 picomolar (pM). In some embodiments of the invention, the targeted binding agent binds heparanase with a Kd of less than 900 pM. In other embodiments, the targeted binding agent binds heparanase with a Kd of less than 800 pM, 700 pM or 600 pM. In some embodiments of the invention, the targeted binding agent binds heparanase with a Kd of less than 500 pM. In other embodiments, the targeted binding agent binds heparanase with a Kd of less than 400 pM. In still other embodiments, the targeted binding agent binds heparanase with a Kd of less than 300 pM. In some other embodiments, the targeted binding agent binds heparanase with a Kd of less than 200 pM. In some other embodiments, the targeted binding agent binds heparanase with a Kd of less than 100 pM. In some other embodiments, the targeted binding agent binds heparanase with a Kd of less than 90 pM, 80 pM, 70 pM, 60 pM, 55pM or 50pM. In some other embodiments, the targeted binding agent binds heparanase with a Kd of less than 60 pM. In some other embodiments, the targeted binding agent binds heparanase with a Kd of less than 55 pM. The Kd may be assessed using a method described herein or known to one of skill in the art (e.g., a BIAcore assay, ELISA) (Biacore International AB, Uppsala, Sweden). Targeted binding agents of the invention have considerably improved binding affinities for heparanase in comparison with the antibodies reported in the prior art.

In one embodiment, the invention includes antibodies that specifically bind to mouse or cynomolgus heparanase with very high affinities (Kd). In some embodiments of the invention, the targeted binding agent binds mouse or cynomolgus heparanase with a binding affinity (Kd) of less than 5 nanomolar (nM). In other embodiments, the targeted binding agent binds with a Kd of less than 4 nM, 3 nM, 2.5 nM, 2 nM or 1 nM. In some embodiments of the invention, the targeted binding agent binds mouse or cynomolgus heparanase with a Kd of less than 950 picomolar (pM). In some embodiments of the invention, the targeted binding agent binds mouse or cynomolgus heparanase with a Kd of less than 900 pM. In other embodiments, the targeted binding agent binds mouse or cynomolgus heparanase with a Kd of less than 800 pM, 700 pM or 600 pM. In some embodiments of the invention, the targeted binding agent binds mouse or cynomolgus heparanase with a Kd of less than 500 pM. In other embodiments, the targeted binding agent binds mouse or cynomolgus heparanase with a Kd of less than 400 pM. In still other embodiments, the targeted binding agent binds mouse or cynomolgus heparanase with a Kd of less than 300 pM. In some other embodiments, the targeted binding agent binds mouse or cynomolgus heparanase with a Kd of less than 200 pM. In some other embodiments, the targeted binding agent binds mouse or cynomolgus heparanase with a Kd of less than 100 pM. In some other embodiments, the targeted binding agent binds mouse or cynomolgus heparanase with a Kd of less than 90 pM, N pM, 70 pM, 60 pM, 55 pM or 50 pM. In some other embodiments, the targeted binding agent binds mouse or cynomolgus heparanase with a Kd of less than 60 pM. In some other embodiments, the targeted binding agent binds mouse or cynomolgus heparanase with a Kd of less than 55 pM.

The binding properties of the targeted binding agent or antibody of the invention may also be measured by reference to the dissociation or association rates (k_{off} and kₒₙ respectively).

In one embodiment of the invention, a targeted binding agent or an antibody may have an kₒₙ rate (antibody (Ab)+antigen (Ag)^{k}^{ₒₙ}→Ab−Ag) of at least 10⁴ M⁻¹s⁻¹, at least 5×10⁴ M⁻¹s⁻¹, at least 10⁵ M⁻¹s⁻¹, at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment of the invention, targeted binding agent or an antibody may have a k_{off} rate ((Ab−Ag)^{k}^{_{off}}→antibody (Ab)+antigen (Ag)) of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, less than 5×10⁻⁴ s⁻¹, less than 10⁻⁴ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰ s⁻¹.

In another embodiment of the invention, the targeted binding agent at a concentration of 1000 μg/ml, or less, inhibits the degradation of 24 nM heparan sulfate-cryptate (HS-cryptate) mediated by 0.2 nM heparanase, by at least 40%. In another embodiment of the invention, the targeted binding agent at a concentration of 100 μg/ml, or less, inhibits the degradation of 24 nM HS-cryptate mediated by 0.2 nM heparanase, by at least 40%. In another embodiment of the invention, the targeted binding agent at a concentration of 10 μg/ml, or less, inhibits the degradation of 24 nM HS-cryptate mediated by 0.2 nM heparanase, by at least 40%. In another embodiment of the invention, the targeted binding agent at a concentration of 1 μg/ml, or less, inhibits the degradation of 24 nM HS-cryptate mediated by 0.2 nM heparanase, by at least 40%. In another embodiment of the invention, the targeted binding agent at a concentration of 0.1 μg/ml, or less, inhibits the degradation of 24 nM HS-cryptate mediated by 0.2 nM heparanase, by at least 40%.

In another embodiment of the invention, the targeted binding agent at a concentration of 1000 μg/ml, or less, inhibits the degradation of 24 nM HS-cryptate to mediated by 0.2 nM heparanase, by at least 50%. In another embodiment of the invention, the targeted binding agent at a concentration of 100 μg/ml, or less, inhibits the degradation of 24 nM HS-cryptate mediated by 0.2 nM heparanase, by at least 50%. In another embodiment of the invention, the targeted binding agent at a concentration of 10 μg/ml, or less, inhibits the degradation of 24 nM HS-cryptate mediated by 0.2 nM heparanase, by at least 50%. In another embodiment of the invention, the targeted binding agent at a concentration of 1 μg/ml, or less, inhibits the degradation of 24 nM HS-cryptate mediated by 0.2 nM heparanase, by at least 50%. In another embodiment of the invention, the targeted binding agent at a concentration of 0.1 μg/ml, or less, inhibits the degradation of 24 nM HS-cryptate mediated by 0.2 nM heparanase, by at least 50%.

In another embodiment of the invention, the targeted binding agent at a concentration of 1000 μg/ml, or less, inhibits the degradation of 24 nM HS-cryptate mediated by 0.2 nM heparanase, by at least 90%. In another embodiment of the invention, the targeted binding agent at a concentration of 100 μg/ml, or less, inhibits the degradation of 24 nM HS-cryptate mediated by 0.2 nM heparanase, by at least 90%. In another embodiment of the invention, the targeted binding agent at a concentration of 10 μg/ml, or less, inhibits the degradation of 24 nM HS-cryptate mediated by 0.2 nM heparanase, by at least 90%. In another embodiment of the invention, the targeted binding agent at a concentration of 1 μg/ml, or less, inhibits the degradation of 24 nM HS-cryptate mediated by 0.2 nM heparanase, by at least 90%. In another embodiment of the invention, the targeted binding agent at a concentration of 0.1 μg/ml, or less, inhibits the degradation of 24 nM HS-cryptate mediated by 0.2 nM heparanase, by at least 90%.

In one embodiment of the invention, the targeted binding agent has an IC50 of less than 1 μg/ml in inhibiting the degradation of 24 nM HS-cryptate mediated by 0.2 nM heparanase. In one embodiment of the invention, the targeted binding agent has an IC50 of less than 0.5 μg/ml, 0.4 μg/ml, 0.3 μg/ml, 0.2 μg/ml, 0.1 μg/ml, 0.05 μg/ml or 0.0.3 μg/ml in inhibiting the degradation of 24 nM HS-cryptate mediated by 0.2 nM heparanase.

In one embodiment of the invention, the targeted binding agent has an IC90 of less than 10 μg/ml in inhibiting the degradation of 24 nM HS-cryptate mediated by 0.2 nM heparanase. In one embodiment of the invention, the targeted binding agent has an IC90 of less than 5 μg/ml, 4 μg/ml, 3 μg/ml, 2 μg/ml, 1 μg/ml or 0.05 μg/ml in inhibiting the degradation of 24 nM HS-cryptate mediated by 0.2 nM heparanase.

In another embodiment of the invention, culturing of cells in the presence of the targeted binding agent at a concentration of 250 μg/ml, or less, increases the accumulation of precusor heparanase by at least 20%, at least 50% or at least 80% in comparison with cells cultured in the presence of an IgG isotype matched control. In another embodiment of the invention, culturing of cells in the presence of the targeted binding agent at a concentration of 25 μg/ml, or less, increases the accumulation of pro-heparanase by at least 20%, at least 50% or at least 80% in comparison with cells cultured in the presence of an IgG isotype matched control.

A further embodiment is a targeted binding agent or an antibody that specifically binds to heparanase and comprises a sequence comprising any one of the complementarity determining regions (CDR) sequences shown in Table 5. Embodiments of the invention include a targeted binding agent or antibody comprising a sequence comprising: any one of a CDR1, a CDR2 or a CDR3 sequence as shown in Table 5. A further embodiment is a targeted binding agent or an antibody that specifically binds to heparanase and comprises a sequence comprising any two of the CDR sequences shown in Table 5. In another embodiment the targeted binding agent or antibody comprises a sequence comprising a CDR1, a CDR2 and a CDR3 sequence as shown in Table 5. In another embodiment the targeted binding agent or antibody comprises a sequence comprising any one of the CDR sequences shown in Table 6. Embodiments of the invention include a targeted binding agent or antibody comprising a sequence comprising: any one of a CDR1, a CDR2 or a CDR3 sequence as shown in Table 6. In another embodiment the targeted binding agent or antibody comprises a sequence comprising any two of the CDR sequences shown in Table 6. In another embodiment the targeted binding agent or antibody comprises a sequence comprising a CDR1, a CDR2 and a CDR3 sequence as shown in Table 6. In another embodiment the targeted binding agent or antibody may comprise a sequence comprising a CDR1, a CDR2 and a CDR3 sequence as shown in Table 5 and a CDR1, a CDR2 and a CDR3 sequence as shown in Table 6. A further embodiment is a targeted binding agent or an antibody that specifically binds to heparanase and comprises a sequence comprising one of the CDR3 sequences shown in Table 5. A further embodiment is a targeted binding agent or an antibody that specifically binds to heparanase and comprises a sequence comprising one of the CDR3 sequences shown in Table 6. In another embodiment the targeted binding agent or antibody may comprise a sequence comprising a CDR3 sequence as shown in Table 5 and a CDR3 sequence as shown in Table 6.

A further embodiment is a targeted binding agent or an antibody that specifically binds to heparanase and comprises a sequence comprising one of the CDR3 sequences shown in Table 5. In a further embodiment the targeted binding agent or antibody further comprises a sequence comprising a CDR3 sequence as shown in Table 6. In a further embodiment the targeted binding agent or antibody further comprises a sequence comprising a CDR2 and a CDR3 sequence as shown in Table 6. In a further embodiment the targeted binding agent or antibody further comprises a sequence comprising: a CDR1, a CDR2 and a CDR3 sequence as shown in Table 6.

A further embodiment is a targeted binding agent or an antibody that specifically binds to heparanase and comprises a sequence comprising one of the CDR2 and one of the CDR3 sequences shown in Table 5. In a further embodiment the targeted binding agent or antibody further comprises a sequence comprising: a CDR3 sequence as shown in Table 6. In a further embodiment the targeted binding agent or antibody further comprises a sequence comprising: a CDR2 and a CDR3 sequence as shown in Table 6. In a further embodiment the targeted binding agent or antibody further comprises a sequence comprising: a CDR1, a CDR2 and a CDR3 sequence as shown in Table 6.

A further embodiment is a targeted binding agent or an antibody that specifically binds to heparanase and comprises a sequence comprising one of the CDR3 sequences shown in Table 6. In a further embodiment the targeted binding agent or antibody further comprises a sequence comprising a CDR3 sequence as shown in Table 5. In a further embodiment the targeted binding agent or antibody further comprises a sequence comprising a CDR2 and a CDR3 sequence as shown in Table 5. In a further embodiment the targeted binding agent or antibody further comprises a sequence comprising a CDR1, a CDR2 and a CDR3 sequence as shown in Table 5.

A further embodiment is a targeted binding agent or an antibody that specifically binds to heparanase and comprises a sequence comprising one of the CDR2 and one of the CDR3 sequences shown in Table 6. In a further embodiment the targeted binding agent or antibody further comprises a sequence comprising a CDR3 sequence as shown in Table 5. In a further embodiment the targeted binding agent or antibody further comprises a sequence comprising a CDR2 and a CDR3 sequence as shown in Table 5. In a further embodiment the targeted binding agent or antibody further comprises a sequence comprising a CDR1, a CDR2 and a CDR3 sequence as shown in Table 5.

It is noted that those of ordinary skill in the art can readily accomplish CDR determinations. See for example, Kabat et al., Sequences of Proteins of Immunological Interest, Fifth Edition, NIH Publication 91-3242, Bethesda Md. (1991), vols. 1-3. Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (presently available from the Kabat Database Website; see also Nucleic Acids Research, 2000, 28(1), 214-218). Any immunoglobulin sequence can be numbered according to Kabat by performing an alignment with the Kabat reference sequence. Accordingly, the Kabat numbering system provides a uniform system for numbering immunoglobulin chains.

In one embodiment, the targeted binding agent or antibody comprises a sequence comprising any one of the heavy chain sequences (VH) shown in Table 5. In another embodiment the targeted binding agent or antibody comprises a sequence comprising any one of the heavy chain sequences of antibodies 15A1.2, 15A11.1, 10E9.1, 7B9.1, 16G1.1, 16G1.2, 14H12.3 or 16E1.1. Light-chain promiscuity is well established in the art, thus, a targeted binding agent or antibody comprising a sequence comprising any one of the heavy chain sequences of antibodies 15A1.2, 15A11.1, 10E9.1, 7B9.1, 16G1.1, 16G1.2, 14H12.3 or 16E1.1 or another antibody as disclosed herein, may further comprise any one of the light chain sequences (VL) shown in Table 6 or of antibodies 15A1.2, 15A11.1, 10E9.1, 7B9.1, 16G1.1, 16G1.2, 14H12.3 or 16E1.1, or other antibody as disclosed herein. In another embodiment the targeted binding agent or antibody comprises a sequence comprising any one of the heavy chain sequences of antibodies 15A1.2, 15A11.1, 10E9.1, 7B9.1, 16G1.1, 16G1.2, 14H12.3 or 16E1.1 and further comprising the corresponding light chain sequence of antibody 15A1.2, 15A11.1, 10E9.1, 7B9.1, 16G1.1, 16G1.2, 14H12.3 or 16E1.1.

In one embodiment, the targeted binding agent or antibody comprises a sequence comprising any one of the light chain sequences shown in Table 6. In another embodiment, the targeted binding agent or antibody comprises a sequence comprising any one of the light chain sequences of antibodies 15A1.2, 15A11.1, 10E9.1, 7B9.1, 16G1.1, 16G1.2, 14H12.3 or 16E1.1. In another embodiment the targeted binding agent or antibody comprises a sequence comprising the heavy chain sequence of antibody 15A1.2 and further comprising the light chain sequence of antibody 15A1.2. In another embodiment the targeted binding agent or antibody comprises a sequence comprising the heavy chain sequence of antibody 15A11.1 and further comprising the light chain sequence of antibody 15A11.1. In another embodiment the targeted binding agent or antibody comprises a sequence comprising the heavy chain sequence of antibody 10E9.1 and further comprising the light chain sequence of antibody 10E9.1.

In some embodiments, the targeting binding agent is any one of the monoclonal antibodies as shown in Table 1. In some embodiments, the targeting binding agent is a monoclonal antibody selected from the group consisting of: 15A1.2, 15A11.1 and 10E9.1. In one embodiment, the targeted binding agent comprises one or more of fully human monoclonal antibodies 15A1.2, 15A11.1 or 10E9.1. In certain embodiments, the targeting binding agent is monoclonal antibody 15A1.2. In certain other embodiments, the targeting binding agent is monoclonal antibody 15A11.1. In still other embodiments, the targeting binding agent is monoclonal antibody 10E9.1. In additional embodiments, the targeted binding agent is derivable from any of the foregoing monoclonal antibodies.

In another embodiment, the targeted binding agent comprises one, two, three, four, five, or all six of the CDRs of monoclonal antibody 15A1.2 (Hyb 50-013 Heparanase 15A1.2 deposited with the American Type Culture Collection 10801 University Boulevard, Manassas, Va., 20110-2209 (“ATCC”) on Sep. 11, 2008, ATCC Patent Deposit Designation: PTA-9491).

In another embodiment, the targeted binding agent comprises one, two, three, four, five, or all six of the CDRs of monoclonal antibody 15A11.1 (Hybridoma 50-013 Heparanase 15A11.1 deposited with the American Type Culture Collection (“ATCC”) on Sep. 11, 2008, ATCC Patent Deposit Designation: PTA-9490).

In another embodiment, the targeted binding agent comprises one, two, three, four, five, or all six of the CDRs of monoclonal antibody 10E9.1 (Hyb 50-013 Heparanase 10E9 deposited with the American Type Culture Collection (“ATCC”) on Sep. 11, 2008, ATCC Patent Deposit Designation: PTA-9489).

In one embodiment a targeted binding agent or an antibody may comprise a sequence comprising a heavy chain CDR1 (HCDR1), heavy chain CDR2 (HCDR2) and heavy chain CDR3 (HCDR3) selected from any one of the sequences shown in Table 5. In one embodiment a targeted binding agent or an antibody may comprise a sequence comprising a light chain CDR1 (LCDR1), light chain CDR2 (LCDR2) and light chain CDR3 (LCDR3) selected from any one of the sequences shown in Table 6. In one embodiment a targeted binding agent or an antibody may comprise a sequence comprising a HCDR1, HCDR2 and HCDR3 selected from any one of the sequences shown in Table 5 and a LCDR1, LCDR2 and LCDR3 selected from any one of the sequences shown in Table 6. In one embodiment a targeted binding agent or an antibody may comprise a sequence comprising a HCDR1, HCDR2 and HCDR3 selected from any one of the CDRs of antibodies 15A1.2, 15A11.1, 10E9.1, 7B9.1, 16G1.1, 16G1.2, 14H12.3 or 16E1.1. In one embodiment a targeted binding agent or an antibody may comprise a sequence comprising a LCDR1, LCDR2 and LCDR3 selected from any one of the CDRs of antibodies 15A1.2, 15A11.1, 10E9.1, 7B9.1, 16G1.1, 16G1.2, 14H12.3 or 16E1.1. In one embodiment a targeted binding agent or an antibody may comprise a sequence comprising a HCDR1, HCDR2 and HCDR3 from any one of antibodies 15A1.2, 15A11.1, 10E9.1, 7B9.1, 16G1.1, 16G1.2, 14H12.3 or 16E1.1. In one embodiment a targeted binding agent or an antibody may comprise a sequence comprising a LCDR1, LCDR2 and LCDR3 from any one of antibodies 15A1.2, 15A11.1, 10E9.1, 7B9.1, 16G1.1, 16G1.2, 14H12.3 or 16E1.1. In one embodiment a targeted binding agent or an antibody may comprise a sequence comprising a HCDR1, HCDR2 and HCDR3 from any one of antibodies 15A1.2, 15A11.1, 10E9.1, 7B9.1, 16G1.1, 16G1.2, 14H12.3 or 16E1.1 and a LCDR1, LCDR2 and LCDR3 from any one of antibodies 15A1.2, 15A11.1, 10E9.1, 7B9.1, 16G1.1, 16G1.2, 14H12.3 or 16E1.1.

In another embodiment the targeted binding agent or antibody may comprise a sequence comprising any one of a CDR1, a CDR2 or a CDR3 of any one of the fully human monoclonal antibodies 15A1.2, 15A11.1 or 10E9.1, as shown in Table 5. In another embodiment the targeted binding agent or antibody may comprise a sequence comprising any one of a CDR1, a CDR2 or a CDR3 of any one of the fully human monoclonal antibodies 15A1.2, 15A11.1 or 10E9.1, as shown in Table 6. In one embodiment the targeted binding agent or antibody may comprise a sequence comprising a CDR1, a CDR2 and a CDR3 of any one of fully human monoclonal antibodies 15A1.2, 15A11.1 or 10E9.1, as shown in Table 5. In another embodiment the targeted binding agent or antibody may comprise a sequence comprising a CDR1, a CDR2 and a CDR3 of any one of fully human monoclonal antibodies 15A1.2, 15A11.1 or 10E9.1, as shown in Table 6. In another embodiment the targeted binding agent or antibody may comprise a sequence comprising a CDR1, a CDR2 and a CDR3 of any one of fully human monoclonal antibodies 15A1.2, 15A11.1 or 10E9.1, as shown in Table 5, and a CDR1, a CDR2 and a CDR3 sequence of any one of fully human monoclonal antibodies 15A1.2, 15A11.1 or 10E9.1, as shown in Table 6.

In another embodiment the targeted binding agent or antibody comprises a sequence comprising the CDR1, CDR2 and CDR3 sequence of fully human monoclonal antibody 15A1.2 (SEQ ID NO.:53, SEQ ID NO.:54 and SEQ ID NO.:55, respectively) as shown in Table 5. In another embodiment the targeted binding agent or antibody comprises a sequence comprising the CDR1, CDR2 and CDR3 sequence of fully human monoclonal antibody 15A1.2 (SEQ ID NO.:62, SEQ ID NO.:63 and SEQ ID NO.:64, respectively) as shown in Table 6. In another embodiment the targeted binding agent or antibody comprises a sequence comprising the CDR1, CDR2 and CDR3 sequence of fully human monoclonal antibody 15A1.2 (SEQ ID NO.:53, SEQ ID NO.:54 and SEQ ID NO.:55, respectively) as shown in Table 5 and the CDR1, CDR2 and CDR3 sequence of fully human monoclonal antibody 15A1.2 (SEQ ID NO.:62, SEQ ID NO.:63 and SEQ ID NO.:64, respectively) as shown in Table 6.

In another embodiment the targeted binding agent or antibody comprises a sequence comprising the CDR1, CDR2 and CDR3 sequence of fully human monoclonal antibody 15A11.1 (SEQ ID NO.:50, SEQ ID NO.:51 and SEQ ID NO.:52, respectively) as shown in Table 5. In another embodiment the targeted binding agent or antibody comprises a sequence comprising the CDR1, CDR2 and CDR3 sequence of fully human monoclonal antibody 15A11.1 (SEQ ID NO.:59, SEQ ID NO.:60 and SEQ ID NO.:61, respectively) as shown in Table 6. In another embodiment the targeted binding agent or antibody comprises a sequence comprising the CDR1, CDR2 and CDR3 sequence of fully human monoclonal antibody 15A11.1 (SEQ ID NO.:50, SEQ ID NO.:51 and SEQ ID NO.:52, respectively) as shown in Table 5 and the CDR1, CDR2 and CDR3 sequence of fully human monoclonal antibody 15A11.1 (SEQ ID NO.:59, SEQ ID NO.:60 and SEQ ID NO.:61, respectively) as shown in Table 6.

In another embodiment the targeted binding agent or antibody comprises a sequence comprising the CDR1, CDR2 and CDR3 sequence of fully human monoclonal antibody 10E9.1 (SEQ ID NO.:47, SEQ ID NO.:48 and SEQ ID NO.:49, respectively) as shown in Table 5. In another embodiment the targeted binding agent or antibody comprises a sequence comprising the CDR1, CDR2 and CDR3 sequence of fully human monoclonal antibody 10E9.1 (SEQ ID NO.:56, SEQ ID NO.:57 and SEQ ID NO.:58, respectively) as shown in Table 6. In another embodiment the targeted binding agent or antibody comprises a sequence comprising the CDR1, CDR2 and CDR3 sequence of fully human monoclonal antibody 10E9.1 (SEQ ID NO.:47, SEQ ID NO.:48 and SEQ ID NO.:49, respectively) as shown in Table 5 and the CDR1, CDR2 and CDR3 sequence of fully human monoclonal antibody 10E9.1 (SEQ ID NO.:56, SEQ ID NO.:57 and SEQ ID NO.:58, respectively) as shown in Table 6.

A further embodiment of the invention is a targeted binding agent or antibody comprising a sequence comprising the contiguous sequence spanning the framework regions and CDRs, specifically from FR1 through FR4 or CDR1 through CDR3, of any one of the sequences as shown in Table 5 or Table 6. A further embodiment of the invention is a targeted binding agent or antibody comprising a sequence comprising the contiguous sequence spanning the framework regions and CDRs, specifically from FR1 through FR4 or CDR1 through CDR3, of any one of the sequences as shown in Table 5 and Table 6. In one embodiment the targeted binding agent or antibody comprises a sequence comprising the contiguous sequences spanning the framework regions and CDRs, specifically from FR1 through FR4 or CDR1 through CDR3, of any one of the sequences of monoclonal antibodies 15A1.2, 15A11.1 or 10E9.1, as shown in Table 5 or Table 6. A further embodiment of the invention is a targeted binding agent or antibody comprising a sequence comprising the contiguous sequence spanning the framework regions and CDRs, specifically from FR1 through FR4 or CDR1 through CDR3, of any one of the sequences of monoclonal antibodies 15A1.2, 15A11.1 or 10E9.1 as shown in Table 5 and Table 6.

One embodiment provides a targeted binding agent or antibody, or antigen-binding portion thereof, wherein the agent or antibody, or antigen-binding portion thereof, comprises a sequence comprising SEQ ID NO.:2, SEQ ID NO.:4, SEQ ID NO.:6, SEQ ID NO.:8, SEQ ID NO.:10 or SEQ ID NO.:12.

One embodiment provides a targeted binding agent or antibody, or antigen-binding portion thereof, wherein the agent or antibody, or antigen-binding portion thereof, comprises a heavy chain sequence comprising the sequence of SEQ ID NO.:6. In one embodiment, the targeted binding agent or antibody, or antigen-binding portion thereof, further comprises a light chain sequence comprising the sequence of SEQ ED NO.:8. In another embodiment the targeted binding agent or antibody, or antigen-binding portion thereof, comprises a heavy chain variable domain having at least 90% identity to the amino acid of SEQ ID NO:6 and comprises a light chain variable domain having at least 90% identity to the amino acid sequence of SEQ ID NO:8.

In another embodiment the targeted binding agent or antibody, or antigen-binding portion thereof, comprises a heavy chain sequence comprising the sequence of SEQ ID NO.:2. In one embodiment, the targeted binding agent or antibody, or antigen-binding portion thereof, further comprises a light chain sequence comprising the sequence of SEQ ID NO.:4.

In one embodiment the targeted binding agent or antibody, or antigen-binding portion thereof, comprises a heavy chain variable domain having at least 90% identity to the amino acid of SEQ ID NO:2 and comprises a light chain variable domain having at least 90% identity to the amino acid sequence of SEQ ID NO:4.

In another embodiment the targeted binding agent or antibody, or antigen-binding portion thereof, comprises a heavy chain sequence comprising the sequence of SEQ ID NO.:10. In another embodiment, the targeted binding agent or antibody, or antigen-binding portion thereof, further comprises a light chain sequence comprising the sequence of SEQ ID NO.:12.

In another embodiment the targeted binding agent or antibody, or antigen-binding portion thereof, comprises a heavy chain variable domain having at least 90% identity to the amino acid of SEQ ID NO:10 and comprises a light chain variable domain having at least 90% identity to the amino acid sequence of SEQ ID NO:12.

In one embodiment, the targeted binding agent or antibody comprises variants or derivatives of the CDRs disclosed herein, the contiguous sequences spanning the framework regions and CDRs (specifically from FR1 through FR4 or CDR1 through CDR3), the light or heavy chain sequences disclosed herein, or the antibodies disclosed herein. Variants include targeted binding agents or antibodies comprising sequences which have as many as twenty, sixteen, ten, nine or fewer, e.g. one, two, three, four, five or six amino acid additions, substitutions, deletions, and/or insertions in any of the CDR1, CDR2 or CDR3s as shown in Table 5 or Table 6, the contiguous sequences spanning the framework regions and CDRs (specifically from FR1 through FR4 or CDR1 through CDR3) as shown in Table 5 or Table 6, the light or heavy chain sequences disclosed herein, or with the monoclonal antibodies disclosed herein. Variants include targeted binding agents or antibodies comprising sequences which have one, two or three, amino acid additions, substitutions, deletions, and/or insertions in any of the CDR1, CDR2 or CDR3s as shown in Table 5 or Table 6, the contiguous sequences spanning the framework regions and CDRs (specifically from FR1 through FR4 or CDR1 through CDR3) as shown in Table 5 or Table 6, the light or heavy chain sequences disclosed herein, or with the monoclonal antibodies disclosed herein. Variants include targeted binding agents or antibodies comprising sequences which have at least about 60, 70, 80, 85, 90, 95, 98 or about 99% amino acid sequence identity with any of the CDR1, CDR2 or CDR3s as shown in Table 5 or Table 6, the contiguous sequences spanning the framework regions and CDRs (specifically from FR1 through FR4 or CDR1 through CDR3) as shown in Table 5 or Table 6, the light or heavy chain sequences disclosed herein, or with the monoclonal antibodies disclosed herein. The percent identity of two amino acid sequences can be determined by any method known to one skilled in the art, including, but not limited to, pairwise protein alignment. In one embodiment variants comprise changes in the CDR sequences or light or heavy chain sequences disclosed herein that are naturally occurring or are introduced by in vitro engineering of native sequences using recombinant DNA techniques or mutagenesis techniques. Naturally occurring variants include those which are generated in vivo in the corresponding germline nucleotide sequences during the generation of an antibody to a foreign antigen. In one embodiment the derivative may be a heteroantibody, that is an antibody in which two or more antibodies are linked together. Derivatives include antibodies which have been chemically modified. Examples include covalent attachment of one or more polymers, such as water-soluble polymers, N-linked, or O-linked carbohydrates, sugars, phosphates, and/or other such molecules. The derivatives are modified in a manner that is different from naturally occurring or starting antibody, either in the type or location of the molecules attached. Derivatives further include deletion of one or more chemical groups which are naturally present on the antibody.

In some embodiments of the invention, the targeted binding agent or antibody comprises a sequence comprising SEQ ID NO.: 2, wherein SEQ ID NO.: 2 comprises any one of the combinations of residues indicated by each row of Table 7a. In some embodiments of the invention, the targeted binding agent or antibody comprises a sequence comprising SEQ ID NO.: 2, wherein SEQ ID NO.: 2 comprises any one, any two, any three, any four or all five of the germline residues as indicated in Table 7a. In some embodiments of the invention, the targeted binding agent or antibody comprises a sequence comprising SEQ ID NO.: 2, wherein SEQ ID NO.: 2 comprises any one of the combinations of residues indicated by each row of Table 7b. In some embodiments of the invention, the targeted binding agent or antibody comprises a sequence comprising SEQ ID NO.: 2, wherein SEQ ID NO.: 2 comprises any one, any two, any three, any four, any five, any six, any seven, any eight, any nine or all ten of the germline residues as indicated in Table 7b. In some embodiments of the invention, the targeted binding agent or antibody comprises a sequence comprising SEQ ID NO.: 2.

In some embodiments of the invention, the targeted binding agent or antibody comprises a sequence comprising SEQ ID NO.: 2, wherein SEQ ID NO.: 2 comprises any one of the combinations of residues indicated by each row of Table 7a and any one of the combinations of residues indicated by each row of Table 7b. In some embodiments of the invention, the targeted binding agent or antibody comprises a sequence comprising SEQ ID NO.: 2, wherein SEQ ID NO.: 2 comprises any one, any two, any three, any four, any five, any six, any seven, any eight, any nine, any ten, any eleven, any twelve, any thirteen, any fourteen or all fifteen of the germline residues as indicated in Table 7a and Table 7b. In other embodiments, the targeted binding agent or antibody is derived from a germline sequence with VH1-24, D4-17 and JH6B domains, wherein one or more residues has been mutated to yield the corresponding germline residue at that position.

In some embodiments of the invention, the targeted binding agent or antibody comprises a sequence comprising SEQ ID NO.: 4, wherein SEQ ID NO.: 4 comprises any one of the combinations of residues indicated by each row of Table 8. In some embodiments of the invention, the targeted binding agent or antibody comprises a sequence comprising SEQ ID NO.: 4, wherein SEQ ID NO.: 4 comprises any one, any two, any three, any four, any five, any six, any seven, any eight or all nine of the germline residues as indicated in Table 8. In some embodiments of the invention, the targeted binding agent or antibody comprises a sequence comprising SEQ ID NO.: 4. In other embodiments, the targeted binding agent or antibody is derived from a germline sequence with A20 and JK3 domains, wherein one or more residues has been mutated to yield the corresponding germline residue at that position.

In some embodiments of the invention, the targeted binding agent or antibody comprises a sequence comprising SEQ ID NO.: 6, wherein SEQ ID NO.: 6 comprises any one of the combinations of residues indicated by each row of Table 9. In some embodiments of the invention, the targeted binding agent or antibody comprises a sequence comprising SEQ ID NO.: 6, wherein SEQ ID NO.: 6 comprises any one, any two, any three, any four or all five of the germline residues as indicated in Table 9. In some embodiments of the invention, the targeted binding agent or antibody comprises a sequence comprising SEQ ID NO.: 6. In other embodiments, the targeted binding agent or antibody is derived from a germline sequence with VH1-24, D3-3 and JH6B domains, wherein one or more residues has been mutated to yield the corresponding germline residue at that position.

In some embodiments of the invention, the targeted binding agent or antibody comprises a sequence comprising SEQ ID NO.: 8, wherein SEQ ID NO.: 8 comprises any one of the combinations of residues indicated by each row of Table 10. In some embodiments of the invention, the targeted binding agent or antibody comprises a sequence comprising SEQ ID NO.: 8, wherein SEQ ID NO.: 8 comprises any one, any two, any three, any four, any five or all six of the germline residues as indicated in Table 10. In some embodiments of the invention, the targeted binding agent or antibody comprises a sequence comprising SEQ ID NO.: 8. In other embodiments, the targeted binding agent or antibody is derived from a germline sequence with L5 and JK1 domains, wherein one or more residues has been mutated to yield the corresponding germline residue at that position.

In some embodiments of the invention, the targeted binding agent or antibody comprises a sequence comprising SEQ ID NO.: 10, wherein SEQ ID NO.: 10 comprises any one of the combinations of residues indicated by each row of Table 11. In some embodiments of the invention, the targeted binding agent or antibody comprises a sequence comprising SEQ ID NO.: 10, wherein SEQ ID NO.: 10 comprises any one, any two, any three, any four, any five, any six, any seven or all eight of the germline residues as indicated in Table 11. In some embodiments of the invention, the targeted binding agent or antibody comprises a sequence comprising SEQ ID NO.: 10. In other embodiments, the targeted binding agent or antibody is derived from a germline sequence with VH1-24, D4-17 and JH6B domains, wherein one or more residues has been mutated to yield the corresponding germline residue at that position.

In some embodiments of the invention, the targeted binding agent or antibody comprises a sequence comprising SEQ ID NO.: 12, wherein SEQ ID NO.: 12 comprises any one of the combinations of residues indicated by each row of Table 12. In some embodiments of the invention, the targeted binding agent or antibody comprises a sequence comprising SEQ ID NO.: 12, wherein SEQ ID NO.: 12 comprises one of the germline residues as indicated in Table 12. In some embodiments of the invention, the targeted binding agent or antibody comprises a sequence comprising SEQ ID NO.: 12. In other embodiments, the targeted binding agent or antibody is derived from a germline sequence with A20 and JK3 domains, wherein one or more residues has been mutated to yield the corresponding germline residue at that position.

In some embodiments of the invention, the targeted binding agent or antibody comprises a sequence comprising a heavy chain variable domain sequence comprising SEQ ID NO.: 2 and a light chain variable domain sequence comprising SEQ ID NO.: 4. In some embodiments of the invention, the targeted binding agent or antibody comprises a sequence comprising SEQ ID NO.: 2 and SEQ ID NO.: 4.

In some embodiments of the invention, the targeted binding agent or antibody comprises a sequence comprising a heavy chain variable domain sequence comprising SEQ ID NO.: 6 and a light chain variable domain sequence comprising SEQ ID NO.: 8. In some embodiments of the invention, the targeted binding agent or antibody comprises a sequence comprising SEQ ID NO.: 6 and SEQ ID NO.: 8.

In some embodiments of the invention, the targeted binding agent or antibody comprises a sequence comprising a heavy chain variable domain sequence comprising SEQ ID NO.: 10 and a light chain variable domain sequence comprising SEQ ID NO.: 12. In some embodiments of the invention, the targeted binding agent or antibody comprises a sequence comprising SEQ ID NO.: 10 and SEQ ID NO.: 12.

Other embodiments of the invention include human monoclonal antibodies that bind heparanase and comprise a heavy chain sequence derived from a VH1-24 germ line sequence. Other embodiments of the invention include human monoclonal antibodies that bind heparanase and comprise a heavy chain sequence derived from a VH3-33 germ line sequence. Other embodiments of the invention include human monoclonal antibodies that bind heparanase and comprise a heavy chain sequence derived from a JH6B or JH3B germ line sequence.

Other embodiments of the invention include human monoclonal antibodies that bind heparanase and heparanase enzyme activity and comprise a HCDR3 comprising 12 or more amino acid residues. In one embodiment the HCDR3 comprises 13 or more amino acid residues. In one embodiment the HCDR3 comprises 14 or more amino acid residues. In one embodiment the HDR3 comprises 18 amino acid residues.

Other embodiments of the invention include human monoclonal antibodies that bind heparanase and comprise a heavy chain CDR3 comprising 2 or more acidic amino acid residues. In one embodiment, the targeted binding agent or antibody comprises a sequence comprising the heavy chain sequence of antibody 10E9.1 (SEQ ID NO.:2), wherein SEQ ID NO.:2 comprises an amino acid sequence in which:

the amino acid residue at position 28 is T or S;

the amino acid residue at position 34 is M or I;

the amino acid residue at position 58 is T or I;

the amino acid residue at position 60 is Y or F;

the amino acid residue at position 70 is M or L;

the amino acid residue at position 74 is T or I;

the amino acid residue at position 76 is T or L;

the amino acid residue at position 83 is L or V;

the amino acid residue at position 99 is E or is deleted;

the amino acid residue at position 100 is G or is deleted;

the amino acid residue at position 101 is H or deleted;

the amino acid residue at position 103 is Y or S;

the amino acid residue at position 107 is V or is deleted;

the amino acid residue at position 108 is Y or G; and/or

the amino acid residue at position 114 is M or L.

References to residue positions are with respect SEQ ID NO.:2, in particular where an amino acid residue is deleted, the position of subsequent residues are not shifted as a result of such deletion.

In one embodiment, the targeted binding agent or antibody comprises a sequence comprising the light chain sequence of antibody 10E9.1 (SEQ ID NO.:4), wherein SEQ ID NO.:4 comprises an amino acid sequence in which:

the amino acid residue at position 28 is G or D;

the amino acid residue at position 30 is S or R;

the amino acid residue at position 32 is Y or F;

the amino acid residue at position 43 is V or L;

the amino acid residue at position 85 is T or S;

the amino acid residue at position 90 is K or S;

the amino acid residue at position 91 is Y or H;

the amino acid residue at position 94 is A or V; and/or

the amino acid residue at position 107 is K or R.

In one embodiment, the targeted binding agent or antibody comprises a sequence comprising the heavy chain sequence of antibody 15A1.2 (SEQ ID NO.:6), wherein SEQ ID NO.:6 comprises an amino acid sequence in which:

the amino acid residue at position 60 is Y or F;

the amino acid residue at position 99 is G or is deleted;

the amino acid residue at position 106 is L or F;

the amino acid residue at position 108 is W or is deleted; and/or

the amino acid residue at position 110 is M or W.

References to residue positions are with respect SEQ ID NO.:6, in particular where an amino acid residue is deleted, the position of subsequent residues are not shifted as a result of such deletion.

In one embodiment, the targeted binding agent or antibody comprises a sequence comprising the light chain sequence of antibody 15A1.2 (SEQ ID NO.:8), wherein SEQ ID NO.:8 comprises an amino acid sequence in which:

the amino acid residue at position 31 is S or N;

the amino acid residue at position 39 is K or T;

the amino acid residue at position 45 is K or Q;

the amino acid residue at position 50 is A or G;

the amino acid residue at position 92 is N or D; and/or

the amino acid residue at position 96 is W or P.

In one embodiment, the targeted binding agent or antibody comprises a sequence comprising the heavy chain sequence of antibody 15A11.1 (SEQ ID NO.:10), wherein SEQ ID NO.:10 comprises an amino acid sequence in which:

the amino acid residue at position 27 is Y or D;

the amino acid residue at position 40 is A or T;

the amino acid residue at position 72 is E or G;

the amino acid residue at position 99 is E or is deleted;

the amino acid residue at position 100 is G or is deleted;

the amino acid residue at position 105 is S or is deleted;

the amino acid residue at position 106 is E or is deleted; and/or

the amino acid residue at position 107 is E or is deleted.

References to residue positions are with respect SEQ ID NO.:10, in particular where an amino acid residue is deleted, the position of subsequent residues are not shifted as a result of such deletion.

In one embodiment, the targeted binding agent or antibody comprises a sequence comprising the light chain sequence of antibody 15A11.1 (SEQ ID NO.:12), wherein SEQ ID NO.:12 comprises an amino acid sequence in which:

the amino acid residue at position 93 is S or R.

In one embodiment, the targeted binding agent or antibody comprises any one or more of:
(i) a VH CDR1 having an amino acid sequence identical to or comprising 1, 2, or 3 amino acid residue substitutions relative to the Kabat VH CDR1 sequence of monoclonal antibody 10E9.1 (SEQ ID NO.:47);
(ii) a VH CDR2 having an amino acid sequence identical to or comprising 1, 2, or 3 amino acid residue substitutions relative to the Kabat VH CDR2 sequence of monoclonal antibody 10E9.1 (SEQ ID NO.:48);
(iii) a VH CDR3 having an amino acid sequence identical to or comprising 1, 2, or 3 amino acid residue substitutions relative to the Kabat VH CDR3 sequence of monoclonal antibody 10E9.1 (SEQ ID NO.:49);
(iv) a VL CDR1 having an amino acid sequence identical to or comprising 1, 2, or 3 amino acid residue substitutions relative to the Kabat VL CDR1 sequence of monoclonal antibody 10E9.1 (SEQ ID NO.:56);
(v) a VL CDR2 having an amino acid sequence identical to or comprising 1, 2, or 3 amino acid residue substitutions relative to the Kabat VL CDR2 sequence of monoclonal antibody 10E9.1 (SEQ ID NO.:57); and
(vi) a VL CDR3 having an amino acid sequence identical to or comprising 1, 2, or 3 amino acid residue substitutions relative to the Kabat VL CDR3 sequence of monoclonal antibody 10E9.1 (SEQ ID NO.:58).

In another embodiment, the targeted binding agent or antibody comprises any one or more of:
(i) a VH CDR1 having an amino acid sequence identical to or comprising 1, 2, or 3 amino acid residue substitutions relative to the Kabat VH CDR1 sequence of monoclonal antibody 15A1.2 (SEQ ID NO.:53);
(ii) a VH CDR2 having an amino acid sequence identical to or comprising 1, 2, or 3 amino acid residue substitutions relative to the Kabat VH CDR2 sequence of monoclonal antibody 15A1.2 (SEQ ID NO.:54);
(iii) a VH CDR3 having an amino acid sequence identical to or comprising 1, 2, or 3 amino acid residue substitutions relative to the Kabat VH CDR3 sequence of monoclonal antibody 15A1.2 (SEQ ID NO.:55);
(iv) a VL CDR1 having an amino acid sequence identical to or comprising 1, 2, or 3 amino acid residue substitutions relative to the Kabat VL CDR1 sequence of monoclonal antibody 15A1.2 (SEQ ID NO.:62);
(v) a VL CDR2 having an amino acid sequence identical to or comprising 1, 2, or 3 amino acid residue substitutions relative to the Kabat VL CDR2 sequence of monoclonal antibody 15A1.2 (SEQ ID NO.:63); and
(vi) a VL CDR3 having an amino acid sequence identical to or comprising 1, 2, or 3 amino acid residue substitutions relative to the Kabat VL CDR3 sequence of monoclonal antibody 15A1.2 (SEQ ID NO.:64).

In a further embodiment, the targeted binding agent or antibody comprises any one or more of:
(i) a VH CDR1 having an amino acid sequence identical to or comprising 1, 2, or 3 amino acid residue substitutions relative to the Kabat VH CDR1 sequence of monoclonal antibody 15A11.1 (SEQ ID NO.:50);
(ii) a VH CDR2 having an amino acid sequence identical to or comprising 1, 2, or 3 amino acid residue substitutions relative to the Kabat VH CDR2 sequence of monoclonal antibody 15A11.1 (SEQ ID NO.:51);
(iii) a VH CDR3 having an amino acid sequence identical to or comprising 1, 2, or 3 amino acid residue substitutions relative to the Kabat VH CDR3 sequence of monoclonal antibody 15A11.1 (SEQ ID NO.:52);
(iv) a VL CDR1 having an amino acid sequence identical to or comprising 1, 2, or 3 amino acid residue substitutions relative to the Kabat VL CDR1 sequence of monoclonal antibody 15A11.1 (SEQ ID NO.:59);
(v) a VL CDR2 having an amino acid sequence identical to or comprising 1, 2, or 3 amino acid residue substitutions relative to the Kabat VL CDR2 sequence of monoclonal antibody 15A11.1 (SEQ ID NO.:60); and
(vi) a VL CDR3 having an amino acid sequence identical to or comprising 1, 2, or 3 amino acid residue substitutions relative to the Kabat VL CDR3 sequence of monoclonal antibody 15A11.1 (SEQ ID NO.:61).

In one embodiment, the targeted binding agent or antibody comprises any one or more of:
(i) a VH CDR1 having the amino acid sequence of VH CDR1 of monoclonal antibody 10E9.1 (SEQ ID NO.:47);
(ii) a VH CDR2 having the amino acid sequence of VH CDR2 of monoclonal antibody 10E9.1 (SEQ ID NO.:48);
(iii) a VH CDR3 having the amino acid sequence of VH CDR3 of monoclonal antibody 10E9.1 (SEQ ID NO.:49);
(iv) a VL CDR1 having the amino acid sequence of VL CDR1 of monoclonal antibody 10E9.1 (SEQ ID NO.:56);
(v) a VL CDR2 having the amino acid sequence of VL CDR2 of monoclonal antibody 10E9.1 (SEQ ID NO.:57); and
(vi) a VL CDR3 having the amino acid sequence of VL CDR3 of monoclonal to antibody 10E9.1 (SEQ ID NO.:58).

In another embodiment, the targeted binding agent or antibody comprises any one or more of:
(i) a VH CDR1 having the amino acid sequence of the VH CDR1 of monoclonal antibody 15A1.2 (SEQ ID NO.:53);
(ii) a VH CDR2 having the amino acid sequence of the VH CDR2 of monoclonal antibody 15A1.2 (SEQ ID NO.:54);
(iii) a VH CDR3 having the amino acid sequence of the VH CDR3 of monoclonal antibody 15A1.2 (SEQ ID NO.:55);
(iv) a VL CDR1 having the amino acid sequence of the VL CDR1 of monoclonal antibody 15A1.2 (SEQ ID NO.:62);
(v) a VL CDR2 having the amino acid sequence of the VL CDR2 of monoclonal antibody 15A1.2 (SEQ ID NO.:63); and
(vi) a VL CDR3 having the amino acid sequence of the VL CDR3 of monoclonal antibody 15A1.2 (SEQ ID NO.:64).

In a further embodiment, the targeted binding agent or antibody comprises any one or more of
(i) a VH CDR1 having the amino acid sequence of the VH CDR1 of monoclonal antibody 15A11.1 (SEQ ID NO.:50);
(ii) a VH CDR2 having the amino acid sequence of the VH CDR2 of monoclonal antibody 15A11.1 (SEQ ID NO.:51);
(iii) a VH CDR3 having the amino acid sequence of the VH CDR3 of monoclonal antibody 15A11.1 (SEQ ID NO.:52);
(iv) a VL CDR1 having the amino acid sequence of the VL CDR1 of monoclonal antibody 15A11.1 (SEQ ID NO.:59);
(v) a VL CDR2 having the amino acid sequence of the VL CDR2 of monoclonal antibody 15A11.1 (SEQ ID NO.:60); and
(vi) a VL CDR3 having the amino acid sequence of the VL CDR3 of monoclonal antibody 15A11.1 (SEQ ID NO.:61).

In one embodiment, the targeted binding agent or antibody comprises a VH chain domain comprising three CDRs and a VL chain domain comprising three CDRs, wherein the VH CDR3 has an amino acid sequence selected from
(i) VH CDR3 of monoclonal antibody 10E9.1 (SEQ ID NO.:49);
(ii) VH CDR3 of monoclonal antibody 15A1.2 (SEQ ID NO.:55); or
(iii) VH CDR3 of monoclonal antibody 15A11.1 (SEQ ID NO.:52).

In one embodiment, the targeted binding agent or antibody comprises a VH chain domain comprising three CDRs and a VL chain domain comprising three CDRs, wherein the VL CDR3 has an amino acid sequence selected from
(iv) VL CDR3 of monoclonal antibody 10E9.1 (SEQ ID NO.:58);
(v) VL CDR3 of monoclonal antibody 15A1.2 (SEQ ID NO.:64); or
(iii) VL CDR3 of monoclonal antibody 15A11.1 (SEQ ID NO.:61).

In one embodiment, the targeted binding agent or antibody comprises a VH chain domain comprising three CDRs and a VL chain domain comprising three CDRs, wherein the three CDRs of the VH chain domain comprise
(i) a VH CDR1 having the amino acid sequence of the VH CDR1 of monoclonal antibody 10E9.1 (SEQ ID NO.:47);
(ii) a VH CDR2 having the amino acid sequence of the VH CDR2 of monoclonal antibody 10E9.1 (SEQ ID NO.:48); and
(iii) a VH CDR3 having the amino acid sequence of the VH CDR3 of monoclonal antibody 10E9.1 (SEQ ID NO.:49).

In another embodiment, the targeted binding agent or antibody comprises a VH chain domain comprising three CDRs and a VL chain domain comprising three CDRs, wherein the three CDRs of the VH chain domain comprise
(i) a VH CDR1 having the amino acid sequence of the VH CDR1 of monoclonal antibody 15A1.2 (SEQ ID NO.:53);
(ii) a VH CDR2 having the amino acid sequence of the VH CDR2 of monoclonal antibody 15A1.2 (SEQ ID NO.:54); and
(iii) a VH CDR3 having the amino acid sequence of the VH CDR3 of monoclonal antibody 15A 1.2 (SEQ ID NO.:55).

In a further embodiment, the targeted binding agent or antibody comprises a VH chain domain comprising three CDRs and a VL chain domain comprising three CDRs, wherein the three CDRs of the VH chain domain comprise
(i) a VH CDR1 having the amino acid sequence of the VH CDR1 of monoclonal antibody 15A11.1 (SEQ ID NO.:50);
(ii) a VH CDR2 having the amino acid sequence of the VH CDR2 of monoclonal antibody 15A11.1 (SEQ ID NO.:51); and
(iii) a VH CDR3 having the amino acid sequence of the VH CDR3 of monoclonal antibody 15A11.1 (SEQ ID NO.:52).

In one embodiment the targeted binding agent or antibody comprises a VH chain domain comprising three CDRs and a VL chain domain comprising three CDRs, wherein the three CDRs of the VL chain domain comprise:
(i) a VL CDR1 having the amino acid sequence of the VL CDR1 of monoclonal antibody 10E9.1 (SEQ ID NO.:56);
(ii) a VL CDR2 having the amino acid sequence of the VL CDR2 of monoclonal antibody 10E9.1 (SEQ ID NO.:57); and
(iii) a VL CDR3 having the amino acid sequence of the VL CDR3 of monoclonal antibody 10E9.1 (SEQ ID NO.:58).

In one embodiment the targeted binding agent or antibody comprises a VH chain domain comprising three CDRs and a VL chain domain comprising three CDRs, wherein the three CDRs of the VL chain domain comprise:
(i) a VL CDR1 having the amino acid sequence of the VL CDR1 of monoclonal antibody 15A1.2 (SEQ ID NO.:62);
(ii) a VL CDR3 having the amino acid sequence of the VL CDR2 of monoclonal antibody 15A1.2 (SEQ ID NO.:63); and
(iii) a VL CDR3 having the amino acid sequence of the VL CDR3 of monoclonal antibody 15A1.2 (SEQ ID NO.:64).

In one embodiment the targeted binding agent or antibody comprises a VH chain domain comprising three CDRs and a VL chain domain comprising three CDRs, wherein the three CDRs of the VL chain domain comprise:
(i) a VL CDR1 having the amino acid sequence of the VL CDR1 of monoclonal antibody 15A11.1 (SEQ ID NO.:59);
(ii) a VL CDR2 having the amino acid sequence of the VL CDR2 of monoclonal antibody 15A11.1 (SEQ ID NO.:60); and
(iii) a VL CDR3 having the amino acid sequence of the VL CDR3 of monoclonal antibody 15A11.1 (SEQ ID NO.:61).

A further embodiment of the invention is a targeted binding agent or antibody which competes for binding to heparanase with the targeted binding agent or antibodies of the invention. In another embodiment of the invention there is an antibody which competes for binding to heparanase with the targeted binding agent or antibodies of the invention. In another embodiment the targeted binding agent or antibody competes for binding to heparanase with any one of fully human monoclonal antibodies 15A1.2, 15A11.1 or 10E9.1. “Competes” indicates that the targeted binding agent or antibody competes for binding to heparanase with any one of fully human monoclonal antibodies 15A1.2, 15A11.1 or 10E9.1, i.e. competition is unidirectional.

Embodiments of the invention include a targeted binding agent or antibody which cross competes with any one of fully human monoclonal antibodies 15A1.2, 15A11.1 or 10E9.1 for binding to heparanase. “Cross competes” indicates that the targeted binding agent or antibody competes for binding to heparanase with any one of fully human monoclonal antibodies 15A1.2, 15A11.1 or 10E9.1, and vice versa, i.e. competition is bidirectional.

A further embodiment of the invention is a targeted binding agent or antibody that binds to the same epitope or epitopes on heparanase as the targeted binding agent or antibodies of the invention. Embodiments of the invention also include a targeted binding agent or antibody that binds to the same epitope or epitopes on heparanase as any one of fully human monoclonal antibodies 15A1.2, 15A11.1 or 10E9.1.

In one embodiment, the targeted binding agent is a bispecific antibody. A bispecific antibody is an antibody that has binding specificity for at least two different epitopes on the same or on different proteins. Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.)

Embodiments of the invention described herein relate to monoclonal antibodies that specifically bind heparanase and affect heparanase function. Other embodiments relate to fully human antibodies that specifically bind heparanase and preparations thereof with desirable properties from a therapeutic perspective, including high binding affinity for heparanase, the ability to inhibit heparan sulfate degradation in vitro, high selectivity for inhibition of heparanase signaling, low toxicity, the ability to block heparanase ligands from binding to heparanase, the ability to inhibit heparanase-induced proliferative, angiogenic, cell adhesion or invasion-related diseases include neoplastic diseases, and/or the ability to inhibit tumour cell growth in vitro and in vivo. Still other embodiments relate to fully human antibodies that specifically bind heparanase and preparations thereof that do not result in a significant Human Anti-Chimeric Antibody (HACA) response, thereby allowing for repeated administration.

In one embodiment of the invention there is provided nucleic acid molecule encoding any of the targeted binding agents or antibodies of the invention. In one embodiment is a nucleic acid molecule encoding the light chain or the heavy chain of an antibody of the invention. In one embodiment, the nucleic acid molecule encodes the light chain or the heavy chain of a fully human monoclonal antibody. In one embodiment, the nucleic acid molecule encodes the light chain or the heavy chain of any one of the fully human monoclonal antibodies 15A1.2, 15A11.1, and 10E9.1. In another embodiment, the nucleic acid molecule encodes the light chain and the heavy chain of any one of the fully human monoclonal antibodies 15A1.2, 15A11.1, and 10E9.1. The invention also encompasses polynucleotides that hybridize under stringent or lower stringency hybridization conditions, as defined herein, to polynucleotides that encode any of the targeted binding agents or antibodies described herein.

In another embodiment of the invention there is provided a vector comprising a nucleic acid molecule or molecules as described hereinabove, wherein the vector encodes a targeted binding agent as described hereinabove. In one embodiment of the invention there is provided a vector comprising a nucleic acid molecule or molecules as described hereinabove, wherein the vector encodes a light chain and/or a heavy chain of an antibody as defined hereinabove. In one embodiment, the vector comprises a nucleic acid molecule encoding the light chain and/or the heavy chain of a fully human monoclonal antibody. In one embodiment, the vector comprises a nucleic acid molecule encoding the light chain or the heavy chain of any one of the fully human monoclonal antibodies 15A1.2, 15A11.1, and 10E9.1. In another embodiment, the vector comprises a nucleic acid molecule encoding the light chain and the heavy chain of any one of the fully human monoclonal antibodies 15A1.2, 15A11.1, and 10E9.1.

In a further embodiment there is provided a host cell transformed with any of the nucleic acid molecules as described hereinabove. In another embodiment of the invention there is provided a host cell comprising the vector comprising the nucleic acid molecule as described hereinabove. In one embodiment the host cell may comprise more than one vector.

As known in the art, antibodies can advantageously be, for example, polyclonal, oligoclonal, monoclonal, chimeric, humanised, and/or fully human antibodies.

It will be appreciated that embodiments of the invention are not limited to any particular form of an antibody or method of generation or production. In some embodiments of the invention, the targeted binding agent is a binding fragment of a fully human monoclonal antibody. For example, the targeted binding agent can be a full-length antibody (e.g., having an intact human Fc region) or an antibody binding fragment (e.g., a Fab, Fab′ or F(ab′)₂, Fv, dAb or other well known antibody fragment, as described in more detail below). In addition, the antibodies can be single-domain antibodies such as camelid or human single VH or VL domains that bind to heparanase, such as a dAb fragment.

Embodiments of the invention described herein also provide cells for producing these antibodies. Examples of cells include hybridomas, or recombinantly created cells, such as Chinese hamster ovary (CHO) cells, variants of CHO cells (for example DG44) and NS0 cells that produce antibodies against heparanase. Additional information about variants of CHO cells can be found in Andersen and Reilly (2004) Current Opinion in Biotechnology 15, 456-462 which is incorporated herein in its entirety by reference. The antibody can be manufactured from a hybridoma that secretes the antibody, or from a recombinantly engineered cell that has been transformed or transfected with a gene or genes encoding the antibody.

In addition, one embodiment of the invention is a method of producing a targeted binding agent or an antibody of the invention by culturing host cells under conditions wherein a nucleic acid molecule is expressed to produce the targeted binding agent or antibody followed by recovery of the targeted binding agent or antibody. In one embodiment is a method of producing an antibody of the invention by culturing host cells under conditions wherein a nucleic acid molecule is expressed to produce the antibody, followed by recovery of the antibody.

It should be realised that embodiments of the invention also include any nucleic acid molecule which encodes an antibody or fragment of an antibody of the invention including nucleic acid sequences optimised for increasing yields of antibodies or fragments thereof when transfected into host cells for antibody production.

A further embodiment herein includes a method of producing antibodies that specifically bind to heparanase and inhibit the biological activity of heparanase, by immunising a mammal with an immunogen selected from; cells expressing heparanase, isolated cell membranes containing heparanase, purified heparanase, or a fragment thereof, and/or one or more orthologous sequences or fragments thereof, followed by isolating antibodies that specifically bind to heparanase.

A further embodiment herein includes a method of producing antibodies that specifically bind to heparanase and inhibit heparanase enzyme activity, by immunising a mammal with an immunogen selected from; cells expressing heparanase, isolated cell membranes containing heparanase, purified heparanase, or a fragment thereof, and/or one or more orthologous sequences or fragments thereof, followed by isolating antibodies that specifically bind to heparanase.

Other embodiments are based upon the generation and identification of isolated antibodies that bind specifically to heparanase and inhibit the biological activity of heparanase. Other embodiments are based upon the generation and identification of isolated antibodies that bind specifically to heparanase and inhibit heparanase enzyme activity. Heparanase is expressed on a number of tumour types. Heparanase is also expressed at elevated levels in cell proliferation, angiogenic and invasion-related diseases, such as neoplastic diseases. Antibodies that specifically bind to heparanase can prevent heparanase-induced tumour cell adhesion, motility, invasion, metastasis, tumour growth, angiogenesis and other desired effects.

In addition, the antibody can be manufactured from a hybridoma that secretes the antibody, or from a recombinantly engineered cell that has been transformed or transfected with a gene or genes encoding the antibody.

In one embodiment there is a hybridoma that produces the targeted binding agent or antibody of the invention. In one embodiment there is a hybridoma that produces the light chain and/or the heavy chain of an antibody of the invention. In one embodiment the hybridoma may produce a light chain and/or a heavy chain of a fully human monoclonal antibody. In another embodiment, the hybridoma produces the light chain and/or the heavy chain of the fully human monoclonal antibody 15A1.2, 15A11.1, and 10E9.1. Alternatively the hybridoma may produce an antibody that binds to the same epitope or epitopes as fully human monoclonal antibody 15A1.2, 15A11.1, and 10E9.1. Alternatively the hybridoma may produce an antibody that competes for binding to heparanase with fully human monoclonal antibody 15A1.2, 15A11.1, and 10E9.1. Alternatively the hybridoma may produce an antibody that cross-competes for binding to heparanase with fully human monoclonal antibody 15A1.2, 15A11.1, and 10E9.1.

In other embodiments the invention provides compositions, including a targeted binding agent or antibody of the invention or binding fragment thereof, and a pharmaceutically acceptable carrier.

Still further embodiments of the invention include methods of treating a proliferative, angiogenic, cell adhesion or invasion-related disease in an animal by administering to the animal a therapeutically effective dose of a targeted binding agent of the invention. In certain embodiments the method further comprises selecting an animal in need of treatment for a proliferative, angiogenic, cell adhesion or invasion-related disease, and administering to the animal a therapeutically effective dose of a targeted binding agent of the invention. In certain embodiments, the animal is human. In certain embodiments, the targeted binding agent is an antibody of the invention and may be selected from the group consisting of 15A1.2, 15A11.1 or 10E9.1.

Still further embodiments of the invention include methods of inhibiting heparanase-induced cell proliferation, angiogenesis, cell adhesion and/or invasion-related disease in an animal by administering to the animal a therapeutically effective dose of a targeted binding agent of the invention. In certain embodiments the method further comprises selecting an animal in need of treatment for heparanase induced cell proliferation, angiogenesis, cell adhesion and/or invasion-related disease, and administering to said animal a therapeutically effective dose of a targeted binding agent of the invention. In certain embodiments, the animal is human. In certain embodiments, the targeted binding agent is an antibody of the invention and may be selected from the group consisting of 15A1.2, 15A11.1 or 10E9.1.

Still further embodiments of the invention include methods of inhibiting tumour cell adhesion, motility, invasion, cellular metastasis, tumour growth or angiogenesis in an animal by administering to the animal a therapeutically effective dose of a targeted binding agent of the invention. In certain embodiments the method further comprises selecting an animal in need of treatment for tumour cell adhesion, motility, invasion, cellular metastasis, tumour growth or angiogenesis, and administering to the animal a therapeutically effective dose of a targeted binding agent of the invention. In certain embodiments, the animal is human. In certain embodiments, the targeted binding agent is an antibody of the invention and may be selected from the group consisting of 15A1.2, 15A11.1 or 10E9.1.

Still further embodiments of the invention include methods of treating an animal suffering from a neoplastic disease by administering to the animal a therapeutically effective dose of a targeted binding agent of the invention. In certain embodiments the method further comprises selecting an animal in need of treatment for a neoplastic disease, and administering to the animal a therapeutically effective dose of a targeted binding agent of the invention.

Still further embodiments of the invention include methods of treating an animal suffering from a non-neoplastic disease by administering to the animal a therapeutically effective dose of a targeted binding agent of the invention. In certain embodiments the method further comprises selecting an animal in need of treatment for a non-neoplastic disease, and administering to the animal a therapeutically effective dose of a targeted binding agent of the invention.

Still further embodiments of the invention include methods of treating an animal suffering from a malignant tumour by administering to the animal a therapeutically effective dose of a targeted binding agent of the invention. In certain embodiments the method further comprises selecting an animal in need of treatment for a malignant tumour, and administering to the animal a therapeutically effective dose of a targeted binding agent of the invention.

Still further embodiments of the invention include methods of treating an animal suffering from a disease or condition associated with heparanase expression by administering to the animal a therapeutically effective dose of a targeted binding agent of the invention. In certain embodiments the method further comprises selecting an animal in need of treatment for a disease or condition associated with heparanase expression, and administering to the animal a therapeutically effective dose of a targeted binding agent of the invention.

A malignant tumour may be selected from the group consisting of: solid tumours such as melanoma, skin cancers, small cell lung cancer, non-small cell lung cancer, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, endometrial cancer, kidney cancer, colon cancer, pancreatic cancer, esophageal carcinoma, brain/CNS cancers, head and neck cancers, neuronal cancers, mesothelioma, sarcomas, biliary (cholangiocarcinoma), small bowel adenocarcinoma, pediatric malignancies, epidermoid carcinoma, sarcomas, cancer of the pleural/peritoneal membranes and leukaemia, including acute myeloid leukaemia, acute lymphoblastic leukaemia, and multiple myeloma.

Treatable proliferative, angiogenic, cell adhesion or invasion-related diseases include neoplastic diseases, such as, melanoma, skin cancer, small cell lung cancer, non-small cell lung cancer, salivary gland, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, thyroid cancer, endometrial cancer, kidney cancer, colon cancer, colorectal cancer, pancreatic cancer, esophageal carcinoma, brain/CNS cancers, neuronal cancers, head and neck cancers, mesothelioma, sarcomas, biliary (cholangiocarcinoma), small bowel adenocarcinoma, pediatric malignancies, epidermoid carcinoma, sarcomas, cancer of the pleural/peritoneal membranes and leukaemia, including acute myeloid leukaemia, acute lymphoblastic leukaemia, and multiple myeloma.

Disease-related cell adhesion and/or invasion and/or angiogenesis and/or proliferation may be any abnormal, undesirable or pathological cell adhesion and/or invasion and/or angiogenesis and/or proliferation, for example tumour-related cell adhesion and/or invasion and/or angiogenesis and/or proliferation.

In one embodiment, the neoplastic disease is a solid tumour selected from any one of the following carcinomas of the breast, colon, prostate, stomach, ovary esophagus, pancreas, gallbladder, non-small cell lung, thyroid, endometrium, head and neck, bladder and gliomas.

In other embodiments, the targeted binding agent ameliorates symptoms associated with non-neoplastic disease in a mammal including inflammatory or hyperproliferative disorders, including, but not limited to, inflammatory bowel disease; arthritis, rheumatoid arthritis; cardiovascular disease such as atherosclerosis; renal disease such as nephropathy; and diseases with an angiogenic component including ocular disease, such as ischaemic retinopathy or age-related macular degeneration.

In one embodiment, the targeted agent ameliorates symptoms associated with arthritis or rheumatoid arthritis. Symptoms that may be ameliorated include but are not limited to synovitis, and angiogenesis. In other embodiments, the targeted agent ameliorates symptoms associated with cardiovasular disease. Symptoms that may be ameliorated include but are not limited hyperproliferation and inflammation. In one embodiment, the targeted agent ameliorates symptoms associated with atherosclerosis. Symptoms that may be ameliorated include but are not limited to inflammation and angiogenesis. In some other embodiments, the targeted agent inhibits symptoms associated with renal disease, including but not limited to proteinuric diseases, including diabetic nephropathy. Symptoms that may be ameliorated include but are not limited to vascular leakage and proteinuria. In some other embodiments, the targeted binding agent ameliorates symptoms associated with ocular disease. Symptoms that may be ameliorated include but are not limited to, uncontrolled vascular permeability, vascular leakage and angiogenesis.

In one embodiment the present invention is suitable for use in inhibiting heparanase, in patients with a tumour which is dependent alone, or in part, on heparanase.

Still further embodiments of the invention include use of a targeted binding agent or antibody of the invention in the preparation of a medicament for the treatment of an animal suffering from a proliferative, angiogenic, cell adhesion or invasion-related disease. In certain embodiments the use further comprises selecting an animal in need of treatment for a proliferative, angiogenic, cell adhesion or invasion-related disease.

Still further embodiments of the invention include use of a targeted binding agent or antibody of the invention in the preparation of medicament for the treatment of heparanase-induced cell proliferation, angiogenesis, cell adhesion and/or invasion-related disease in an animal. In certain embodiments the use further comprises selecting an animal in need of treatment for a heparanase-induced proliferative, angiogenic, cell adhesion and/or invasion-related disease.

Still further embodiments of the invention include use of a targeted binding agent or antibody of the invention in the preparation of medicament for the treatment of tumour cell adhesion, motility, invasion, cellular metastasis, tumour growth or angiogenesis in an animal. In certain embodiments the use further comprises selecting an animal in need of treatment for tumour cell adhesion, motility, invasion, cellular metastasis, tumour growth or angiogenesis.

Still further embodiments of the invention include use of a targeted binding agent or antibody of the invention in the preparation of a medicament for the treatment of an animal suffering from a neoplastic disease. In certain embodiments the use further comprises selecting an animal in need of treatment for a neoplastic disease.

Still further embodiments of the invention include use of a targeted binding agent or antibody of the invention in the preparation of a medicament for the treatment of an animal suffering from a non-neoplastic disease. In certain embodiments the use further comprises selecting an animal in need of treatment for a non-neoplastic disease.

Still further embodiments of the invention include use of a targeted binding agent or antibody of the invention in the preparation of a medicament for the treatment of an animal suffering from a malignant tumour. In certain embodiments the use further comprises selecting an animal in need of treatment for a malignant tumour.

Still further embodiments of the invention include use of a targeted binding agent or antibody of the invention in the preparation of a medicament for the treatment of an animal suffering from a disease or condition associated with heparanase expression. In certain embodiments the use further comprises selecting an animal in need of treatment for a disease or condition associated with heparanase expression.

Still further embodiments of the invention include a targeted binding agent or antibody of the invention for use as a medicament for the treatment of an animal suffering from a proliferative, angiogenic, cell adhesion or invasion-related disease.

Still further embodiments of the invention include a targeted binding agent or antibody of the invention for use as a medicament for the treatment of an animal suffering from tumour cell adhesion, motility, invasion, cellular metastasis, tumour growth or angiogenesis in an animal.

Still further embodiments of the invention include a targeted binding agent or antibody of the invention for use as a medicament for the treatment of an animal suffering from a neoplastic disease.

Still further embodiments of the invention include a targeted binding agent or antibody of the invention for use as a medicament for the treatment of an animal suffering from a non-neoplastic disease.

Still further embodiments of the invention include a targeted binding agent or antibody of the invention for use as a medicament for the treatment of an animal suffering from a malignant tumour.

Still further embodiments of the invention include a targeted binding agent or antibody of the invention for use as a medicament for the treatment of an animal suffering from a disease or condition associated with heparanase expression.

In one embodiment treatment of
a proliferative, angiogenic, cell adhesion or invasion-related disease;
a neoplastic disease;
a non-neoplastic disease;
a malignant tumour; or
a disease or condition associated with heparanase expression,
comprises managing, ameliorating, preventing, any of the aforementioned diseases or conditions.

In one embodiment treatment of a neoplastic disease comprises inhibition of tumour growth, tumour growth delay, regression of tumour, shrinkage of tumour, increased time to regrowth of tumour on cessation of treatment, increased time to tumour recurrence, slowing of disease progression.

In one embodiment treatment of a disease or condition associated with heparanase expression comprises inhibiting the growth of cells that express heparanase.

While not being limited to any particular theory, the mechanism of action can include, but is not limited to preventing heparanase-mediated heparan sulfate degradation, thereby inhibiting cell proliferation, adhesion and invasion. Antibodies of the invention may also inhibit the release of heparin binding growth factors such as but not limited to e.g. HGF, HB-EGF, FGF, TGFβ or VEGF, from cell surface or extracellular HSPG binding sites.

In some embodiments, following administration of the targeted binding agent or antibody of the invention, a clearing agent is administered, to remove excess circulating antibody from the blood.

In some embodiments of the invention, the animal to be treated is a human.

In some embodiments of the invention, the targeted binding agent is a fully human monoclonal antibody.

In some embodiments of the invention, the targeted binding agent is selected from the group consisting of fully human monoclonal antibodies 15A1.2, 15A11.1 and 10E9.1.

Embodiments of the invention include a conjugate comprising the targeted binding agent as described herein, and a therapeutic agent. In some embodiments of the invention, the therapeutic agent is a toxin. In other embodiments, the therapeutic agent is a radioisotope. In still other embodiments, the therapeutic agent is a pharmaceutical composition.

In another aspect, a method of selectively killing a cancerous cell in a patient is provided. The method comprises administering a fully human antibody conjugate to a patient. The fully human antibody conjugate comprises an antibody that can bind to heparanase and an agent. The agent is either a toxin, a radioisotope, or another substance that will kill a cancer cell. The antibody conjugate thereby selectively kills the cancer cell.

In another aspect, a method of selectively killing a cancerous cell in a patient is provided. The method comprises administering a fully human antibody conjugate to a patient. The fully human antibody conjugate comprises an antibody that can bind to the extracellular domain of heparanase and an agent. The agent is either a toxin, a radioisotope, or another substance that will kill a cancer cell. The antibody conjugate thereby selectively kills the cancer cell.

In one aspect, a conjugated fully human antibody that specifically binds to heparanase is provided. Attached to the antibody is an agent, and the binding of the antibody to a cell results in the delivery of the agent to the cell. In one embodiment, the above conjugated fully human antibody binds to an extracellular domain of heparanase. In another embodiment, the antibody and conjugated toxin are internalised by a cell that expresses heparanase. In another embodiment, the agent is a cytotoxic agent. In another embodiment, the agent is, for example saporin, or auristatin, pseudomonas exotoxin, gelonin, ricin, calicheamicin or maytansine-based immunoconjugates, and the like. In still another embodiment, the agent is a radioisotope.

The targeted binding agent or antibody of the invention can be administered alone, or can be administered in combination with additional antibodies or chemotherapeutic drugs or radiation therapy. For example, a monoclonal, oligoclonal or polyclonal mixture of heparanase antibodies that block cell adhesion, invasion, angiogenesis or proliferation can be administered in combination with a drug shown to inhibit tumour cell proliferation.

According to another aspect of the invention there is provided a pharmaceutical composition comprising a targeted binding agent of antibody of the invention and a pharmaceutically acceptable carrier.

Another embodiment of the invention includes a method of diagnosing diseases or conditions in which an antibody as disclosed herein is utilised to detect the presence and/or level of heparanase in a patient or patient sample. In one embodiment, the patient sample is blood or blood serum or urine. In further embodiments, methods for the identification of risk factors, diagnosis of disease, and staging of disease is presented which involves the identification of the expression and/or overexpression of heparanase using anti-heparanase antibodies. In some embodiments, the methods comprise administering to a patient a fully human antibody conjugate that selectively binds to heparanase on a cell. The antibody conjugate comprises an antibody that specifically binds to heparanase and a label. The methods further comprise observing the presence of the label in the patient. A relatively high amount of the label will indicate a relatively high risk of the disease and a relatively low amount of the label will indicate a relatively low risk of the disease. In one embodiment, the label is a green fluorescent protein.

The invention further provides methods for assaying for the presence and/or level of heparanase in a patient sample, comprising contacting an antibody as disclosed herein with a biological sample from a patient, and detecting the level of binding between said antibody and heparanase in said sample. In more specific embodiments, the biological sample is blood, plasma or serum.

Another embodiment of the invention includes a method for diagnosing a condition associated with the expression of heparanase in a cell by contacting the serum or a cell with an antibody as disclosed herein, and thereafter detecting the presence of heparanase. In one embodiment the condition can be a proliferative, angiogenic, cell adhesion or invasion-related disease including, but not limited to, a neoplastic disease.

In another embodiment, the invention includes an assay kit for detecting heparanase in mammalian tissues, cells, or body fluids. Such a kit would be useful to screen for heparanase-related diseases. The kit includes a targeted binding agent or antibody of the invention and a means for indicating the reaction of the targeted binding agent or antibody with heparanase, if present. In one embodiment the antibody is a monoclonal antibody. In one embodiment, the antibody that binds heparanase is labeled. In another embodiment the antibody is an unlabeled primary antibody and the kit further includes a means for detecting the primary antibody. In one embodiment, the means for detecting includes a labeled second antibody that is an anti-immunoglobulin. The antibody may be labeled with a marker selected from the group consisting of a fluorochrome, an enzyme, a radionuclide and a radiopaque material.

In some embodiments, the targeted binding agents or antibodies as disclosed herein can be modified to enhance their capability of fixing complement and participating in complement-dependent cytotoxicity (CDC). In other embodiments, the targeted binding agents or antibodies can be modified to enhance their capability of activating effector cells and participating in antibody-dependent cytotoxicity (ADCC). In yet other embodiments, the targeted binding agents or antibodies can be modified both to enhance their capability of activating effector cells and participating in antibody-dependent cytotoxicity (ADCC) and to enhance their capability of fixing complement and participating in complement-dependent cytotoxicity (CDC).

In some embodiments, the targeted binding agents or antibodies as disclosed herein can be modified to reduce their capability of fixing complement and participating in complement-dependent cytotoxicity (CDC). In other embodiments, the targeted binding agents or antibodies can be modified to reduce their capability of activating effector cells and participating in antibody-dependent cytotoxicity (ADCC). In yet other embodiments, the targeted binding agents or antibodies as disclosed herein can be modified both to reduce their capability of activating effector cells and participating in antibody-dependent cytotoxicity (ADCC) and to reduce their capability of fixing complement and participating in complement-dependent cytotoxicity (CDC).

In certain embodiments, the half-life of a targeted binding agent or antibody as disclosed herein and of compositions of the invention is at least about 4 to 7 days. In certain embodiments, the mean half-life of a targeted binding agent or antibody as disclosed herein and of compositions of the invention is at least about 2 to 5 days, 3 to 6 days, 4 to 7 days, 5 to 8 days, 6 to 9 days, 7 to 10 days, 8 to 11 days, 8 to 12, 9 to 13, 10 to 14, 11 to 15, 12 to 16, 13 to 17, 14 to 18, 15 to 19, or 16 to 20 days. In other embodiments, the mean half-life of a targeted binding agent or antibody as disclosed herein and of compositions of the invention is at least about 17 to 21 days, 18 to 22 days, 19 to 23 days, 20 to 24 days, 21 to 25, days, 22 to 26 days, 23 to 27 days, 24 to 28 days, 25 to 29 days, or 26 to 30 days. In still further embodiments the half-life of a targeted binding agent or antibody as disclosed herein and of compositions of the invention can be up to about 50 days. In certain embodiments, the half-lives of antibodies and of compositions of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; and International Publication Nos. WO 98/23289 and WO 97/3461.

In another embodiment, the invention provides an article of manufacture including a container. The container includes a composition containing a targeted binding agent or antibody as disclosed herein, and a package insert or label indicating that the composition can be used to treat cell adhesion, invasion, angiogenesis, and/or proliferation-related diseases, including, but not limited to, diseases characterised by the expression or overexpression of heparanase.

In other embodiments, the invention provides a kit for treating diseases involving the expression of heparanase, comprising a targeted binding agent or antibody as disclosed herein, and instructions to administer the monoclonal antibodies to a subject in need of treatment.

The present invention provides formulation of proteins comprising a variant Fc region. That is, a non naturally occurring Fc region, for example an Fc region comprising one or more non naturally occurring amino acid residues. Also encompassed by the variant Fc regions of present invention are Fc regions which comprise amino acid deletions, additions and/or modifications.

The serum half-life of proteins comprising Fc regions may be increased by increasing the binding affinity of the Fc region for FcRn. In one embodiment, the Fc variant protein has enhanced serum half life relative to comparable molecule.

In another embodiment, the present invention provides an Fc variant, wherein the Fc region comprises at least one non naturally occurring amino acid at one or more positions selected from the group consisting of 239, 330 and 332, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat and at least one non naturally occurring amino acid at one or more positions selected from the group consisting of 252Y, 254T and 256E, as numbered by the EU index as set forth in Kabat.

In another embodiment, the present invention provides an Fc variant, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant, wherein the Fc region comprises at least one non naturally occurring amino acid selected from the group consisting of 234F, 235F, 235Y, 235E and 331 S, as numbered by the EU index as set forth in Kabat. In a further specific embodiment, an Fc variant of the invention comprises the 234F, 235F, and 331S non naturally occurring amino acid residues, as numbered by the EU index as set forth in Kabat. In another specific embodiment, an Fc variant of the invention comprises the 234F, 235Y, and 331 S non naturally occurring amino acid residues, as numbered by the EU index as set forth in Kabat. In another specific embodiment, an Fc variant of the invention comprises the 234F, 235E, and 331 S non naturally occurring amino acid residues, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 234F, 235F, 235Y, 235E and 331 S, as numbered by the EU index as set forth in Kabat; and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant protein formulation, wherein the Fe region comprises at least one non naturally occurring amino acid selected from the group consisting of 234F, 235F, 235Y, 235E and 331 S, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, 235E and 331S, as numbered by the EU index as set forth in Kabat; and at least one non naturally occurring amino acid at one or more positions are selected from the group consisting of 252Y, 254T and 256E, as numbered by the EU index as set forth in Kabat.

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Preferably, site-directed mutagenesis is performed by the overlap-extension PCR method (Higuchi, in “PCR Technology: Principles and Applications for DNA Amplification”, Stockton Press, New York, pp. 61-70 (1989)). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments of the invention, the glycosylation patterns of the antibodies to provided herein are modified to enhance ADCC and CDC effector function. See Shields R L et al., (2002) JBC. 277:26733; Shinkawa T et al., (2003) JBC. 278:3466 and Okazaki A et al., (2004) J. Mol. Biol., 336: 1239. In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

It is also known in the art that the glycosylation of the Fc region can be modified to increase or decrease effector function (see for examples, Umana et al, 1999, Nat Biotechnol 17:176-180; Davies et al., 2001, Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). Accordingly, in one embodiment the Fc regions of the antibodies of the invention comprise altered glycosylation of amino acid residues. In another embodiment, the altered glycosylation of the amino acid residues results in lowered effector function. In another embodiment, the altered glycosylation of the amino acid residues results in increased effector function. In a specific embodiment, the Fc region has reduced fucosylation. In another embodiment, the Fc region is afucosylated (see for examples, U.S. Patent Application Publication No.2005/0226867).

### BRIEF DESCRIPTION OF THE DRAWINGS

FIG. 1 is a line graph illustrating the heparan sulfate degradation assay and the effect of incorporating Dynabeads into the protocol as a means to circumvent the effect of culture medium pH. The x-axis corresponds to the log of the concentration of Heparanase (ng per ml), the y-axis reports the change in fluorescence (% ρF). Recombinant human heparanase (log [Heparanase(ng/ml)], x-axis) was incubated with europium cryptate-heparan sulfate at the concentrations indicated in the presence (solid/open squares) or absence (solid/open circles) of biotin anti-human Fc Dynabeads. The presence of the beads did not alter the cleavage of heparan sulfate by heparanase as reported by the change in fluorescence (% ρF, y-axis).

FIG. 2 is a line graph showing the heparanase inhibitory activity of 121 hybridoma exhaustive supernatants in each of two heparan sulfate cleavage experiments (experiment 1, x-axis; experiment 2, y-axis) expressed as percentage inhibition. Antibodies were captured onto biotin anti-human Fc Dynabeads and incorporated into the heparan sulfate degradation assay.

FIG. 3 is a line graph showing the heparanase inhibitory activity of purified monoclonal antibodies in the heparan sulfate degradation assay. The x-axis corresponds to the concentration of purified antibody (μg per ml), the y-axis reports the change in fluorescence (% ρF). Two unrelated isotype controls are also shown.

FIG. 4 is a line graph showing the heparanase inhibitory activity of three purified antibodies in the heparan sulfate degradation assay. The x-axis corresponds to the log of the concentration of purified antibody (μg per ml), the y-axis reports the change in fluorescence (% ρF). Purified antibodies were diluted over the range 0.05 ng-1000 μg per ml (x-axis) and incubated with 24 nM europium cryptate-heparan sulfate and 0.2 nM recombinant heparanase for 60 minutes at 37° C. Each point is the mean of three measurements.

FIG. 5a-c shows the inhibitory effect of mAbs that specifically bind heparanase on heparanase enzyme activity in conditioned medium obtained from Hek293 cells transfected with human (FIG. 5a), cynomolgus (FIG. 5b) and murine (FIG. 5c) heparanase. The x-axis corresponds to the final concentration of heparanase, the y-axis reports the change in fluorescence (% ρF). Purified antibodies 10E9.1, 15A1.2 and 15A11.1, and an isotype matched irrelevant control antibody, final concentration 1-1000 μg per ml, were added to Hek293 cell conditioned medium to provide a final concentration of heparanase from 1:50-1:200 (x-axis). Europium cryptate-heparan sulfate (final concentration 24nM) was added and incubated at 37° C. for 1 hr. Controls, with no added antibody, provided a baseline for heparan sulfate degradation (% ρF, y-axis), and the assay in the presence of 7 μM suramin as an inhibitor of heparanase provided a positive control for each of the recombinant heparanase enzymes.

FIG. 6 shows the immunoprecipitation of heparanase by 10E9.1 from MCF7 cell lysates. The left hand lane group shows the results obtained with parental cells, and the right hand lane group shows the results obtained with heparanase transfected cells. The lane labelled Input contains non-immunoprecipitated cell lysate.

FIG. 7 shows the effect of a selection of antibodies on inhibition of heparanase expression by anti-heparanase antibodies. Equal protein loading of the lanes is demonstrated by Western blotting for GAPDH (glyceraldehyde phosphate dehydrogenase). Treatment with 10E9.1, 15A1.2 and 15A11.1 resulted in a significant inhibition of heparanase expression compared to untreated or IgG2 treated control.

FIG. 8 shows the effect of anti-heparanase mAb 10E9.1 on the accumulation of precursor (inactive or pro-heparanase) heparanase in conditioned medium after 24 hrs exposure to the antibody. The upper gel shows the results in conditioned media (extracellular), the lower gel shows the results in RIPA extracts (intracellular).

FIG. 9a-c shows the relative binding of anti-heparanase antibody 10E9.1 and control IgG2 antibody to human and murine tumour cells as a percentage of gated events by FACS. FIG. 9a is a bar chart showing the results in HT1080 cells, FIG. 9b is a bar chart showing the effect in HCT116 cells, FIG. 9c is a bar chart showing the effect in B16F10 AP3 cells. The solid-filled bars show the result with cells incubated with control IgG2 antibody. The grey-filled bars show the result with cells incubated with 10E9.1 antibody.

FIG. 10a-b shows the effect of anti-heparanase antibody 10E9.1 and control IgG2 antibody on the degradation of heparan sulfate by cell surface heparanase. The Y axis shows heparanase activity as measured by fluorescence at 670 nm. FIG. 10a is a bar chart showing the results in MCF7 cells, FIG. 10b is a bar chart showing the effect in MCF7-Hpa1 #19 cells. The minimum fluorescence values indicated by the dotted lines indicate the maximum degradation of heparan sulfate. The maximum fluorescence values indicated by the dotted lines indicate the minimum degradation of heparan sulfate. The maximum values observed with 10E9.1 at a concentration of 5 or 25 μg per ml are indicative of inhibition of heparanase activity and likely represent total inhibition of available cell surface heparanase since the fluorescence values plateau between 5 and 25pg per ml of 10E9.1.

FIG. 11a-b shows the effect of anti-heparanase antibodies on subcutaneous xenograft tumour growth. The Y axis shows mean tumour volume in cm3+/−SE against days post selection. The black arrows indicate when the dose of antibody or control was given. FIG. 11a is a line graph showing the effect of 10E9.1 on MCF7-Hpa1 #19 xenograft tumour growth, FIG. 11b is a line graph showing the effect of 10E9.1 on HCT116 xenograft tumour growth. In FIG. 11a the square points represent PBS control 0.1 ml/10 g×1 weekly i.p.; the circles represent 10E9.1 15 mg/kg ×1 weekly i.p.; the triangles represent 10E9.1 7.5 mg/kg ×1 weekly i.p.; the inverted triangles represent IgG2 control 15 mg/kg ×1 weekly i.p. In FIG. 11b the square points represent PBS control 0.1 ml/10 g ×1 weekly i.p.; the circles represent 10E9.1 15 mg/kg ×1 weekly i.p.; the triangles represent IgG2 control 15 mg/kg ×1 weekly i.p.

Further embodiments, features, and the like, regarding targeted binding agent or antibodies of the invention are provided in additional detail below.

### Definitions

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilised in connection with, and techniques of, cell and tissue culture, molecular biology, and protein and oligo- or polynucleotide chemistry and hybridisation described herein are those well known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference. The nomenclatures utilised in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilised in accordance with the present disclosure, the following terms, unless otherwise indicated, shall be understood to have the following meanings:

An antagonist or inhibitor may be a polypeptide, nucleic acid, carbohydrate, lipid, small molecular weight compound, an oligonucleotide, an oligopeptide, RNA interference (RNAi), antisense, a recombinant protein, an antibody, or fragments thereof or conjugates or fusion proteins thereof. For a review of RNAi see Milhavet O, Gary D S, Mattson M P. (Pharmacol Rev. 2003 December; 55(4):629-48. Review) and antisense (see Opalinska J B, Gewirtz A M. (Sci STKE. 2003 Oct. 28; 2003 (206):pe47.)

A compound refers to any small molecular weight compound with a molecular weight of less than about 2000 Daltons.

The term “heparanase” refers to the human heparanase 1 or Hpa1.

The term “neutralising” or “inhibits” when referring to a targeted binding agent, such as an antibody, relates to the ability of said agent to eliminate, reduce, or significantly reduce, the activity of a target antigen. Accordingly, a “neutralising” anti-heparanase antibody of the invention is capable of eliminating or significantly reducing the activity of heparanase. A neutralising, antagonising or inhibiting antibody that specifically binds heparanase may, for example, act by blocking the binding of heparanase to its substrate. By blocking this binding, the degradation of e.g. heparan sulfate or other molecule having heparan sulfate glycosylation is significantly, or completely, eliminated. Ideally, a neutralising antibody against heparanase inhibits cell proliferation, cell adhesion, angiogenesis and invasion. A neutralising, antagonising or inhibiting antibody that specifically binds heparanase may, for example, act by influencing direct modification of heparan sulfate, HSPGs, the release of heparin binding growth factors from ECM or cell surface heparan sulfate binding sites, the modification of cell surface co-receptor activity, and modification or degradation of cell surface HSPGs that have a signalling role and determine cellular proliferative, adhesive and migratory phenotypes.

“Inhibiting the biological activity of heparanase” encompasses an inhibition of heparanase activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% in comparison with the biological activity in the absence of a targeted binding agent or antibody of the invention.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa or lambda light chain immunoglobulin molecules, and vice versa, as well as fragments and analogs thereof. Preferred polypeptides in accordance with the invention may also comprise solely the human heavy chain immunoglobulin molecules or fragments thereof.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally occurring.

The term “control sequence” as used herein refers to polynucleotide sequences that are necessary either to effect or to affect the expression and processing of coding sequences to which they are connected. The nature of such control sequences differs depending upon the host organism; in prokaryotes, such control sequences generally include promoter, ribosomal binding site, and transcription termination sequence; in eukaryotes, generally, such control sequences may include promoters, enhancers, introns, transcription termination sequences, polyadenylation signal sequences, and 5′ and ‘3 untranslated regions. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide, or RNA-DNA hetero-duplexes. The term includes single and double stranded forms of DNA.

The term “oligonucleotide” referred to herein includes naturally occurring, and modified nucleotides linked together by naturally occurring, and non naturally occurring linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridise” referred to herein means to detectably and specifically bind. Polynucleotides, oligonucleotides and fragments thereof selectively hybridise to nucleic acid strands under hybridisation and wash conditions that minimise appreciable amounts of detectable binding to nonspecific nucleic acids. High stringency conditions can be used to achieve selective hybridisation conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

Stringent hybridization conditions include, but are not limited to, hybridization to filter-bound DNA in 6× sodium chloride/sodium citrate (SSC) (0.9 M NaCl/90 mM NaCitrate, pH 7.0) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

Two amino acid sequences are “similar” if there is a partial or complete identity between their sequences. For example, 85% identity means that 85% of the amino acids are identical when the two sequences are aligned for maximum matching. Optimal alignment of the sequences for comparison may be produced, besides manually, by means of the local sequence alignment algorithm of Smith and Waterman, 1981, Ads App. Math. 2, 482, by means of the global sequence alignment algorithm of Needleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.). Gaps (in either of the two sequences being matched) are allowed in maximising matching; gap lengths of 5 or less are preferred with 2 or less being more preferred. Preferably protein sequences are aligned using the BestFit program, which is based on the Smith and Waterman algorithm, with a gap opening penalty of 8, a gap extension penalty of 2 and the Blosum 62 scoring matrix. Two protein sequences (or polypeptide sequences derived from them of at least about 30 amino acids in length) are similar, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably similar if their amino acids are greater than or equal to 50% identical when optimally aligned using the ALIGN program. It should be appreciated that there can be differing regions of similarity within two orthologous sequences. For example, the functional sites of mouse and human orthologues may have a higher degree of similarity than non-functional regions.

The term “corresponds to” is used herein to mean that a polynucleotide sequence is similar (i.e., is partially or completely identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is similar to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention. Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine. As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerised comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognise sequence motifs and structural conformations that may be used to define structural and functional domains in accordance with the antibodies described herein. Glutaminyl and asparaginyl residues are frequently deamidated to the corresponding glutamyl and aspartyl residues, respectively. These residues are deamidated under neutral or basic conditions. The deamidated form of these residues falls within the scope of this invention.

In general, cysteine residues in proteins are either engaged in cysteine-cysteine disulfide bonds or sterically protected from the disulfide bond formation when they are a part of folded protein region. Disulfide bond formation in proteins is a complex process, which is determined by the redox potential of the environment and specialized thiol-disulfide exchanging enzymes (Creighton, Methods Enzymol. 107, 305-329, 1984; Houee-Levin, Methods Enzymol. 353, 35-44,2002). When a cysteine residue does not have a pair in protein structure and is not sterically protected by folding, it can form a disulfide bond with a free cysteine from solution in a process known as disulfide shuffling. In another process known as disulfide scrambling, free cysteines may also interfere with naturally occurring disulfide bonds (such as those present in antibody structures) and lead to low binding, low biological activity and/or low stability.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterises the parent sequence). Examples of art-recognised polypeptide secondary and tertiary structures are described in Proteins, Structures and Molecular Principles (Creighton, Ed, W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference. Additionally, such methods may be used to make amino acid substitutions or deletions of one or more variable region cysteine residues participating in an intrachain disulfide bond to generate antibody molecules lacking one or more intrachain disulfide bonds.

The term “CDR region” or “CDR” is intended to indicate the hypervariable regions of the heavy and light chains of an antibody which confer the antigen-binding specificity to the antibody. CDRs may be defined according to the Kabat system (Kabat, E. A. et al. (1991) Sequences of Proteins of Immunological Interest, 5th Edition. US Department of Health and Human Services, Public Service, NIH, Washington), and later editions. An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognises.

The third CDR of the heavy chain (HCDR3) has a greater size variability (greater diversity essentially due to the mechanisms of arrangement of the genes which give rise to it). It may be as short as 2 amino acids although the longest size known is 26. CDR length may also vary according to the length that can be accommodated by the particular underlying framework. Functionally, HCDR3 plays a role in part in the determination of the specificity of the antibody (Segal et al., PNAS, 71:4298-4302, 1974, Amit et al., Science, 233:747-753, 1986, Chothia et al., J. Mol. Biol., 196:901-917, 1987, Chothia et al., Nature, 342:877-883, 1989, Caton et al., J. Immunol., 144:1965-1968, 1990, Sharon et al., PNAS, 87:4814-4817, 1990, Sharon et al., J. Immunol., 144:4863-4869, 1990, Kabat et al., J. Immunol., 147:1709-1719, 1991).

The term a “set of CDRs” referred to herein comprises CDR1, CDR2 and CDR3. Thus, a set of HCDRs refers to HCDR1, HCDR2 and HCDR3, and a set of LCDRs refers to LCDR1, LCDR2 and LCDR3.

Variants of the VH and VL domains and CDRs of the present invention, including those for which amino acid sequences are set out herein, and which can be employed in targeting binding agents and antibodies for heparanase can be obtained by means of methods of sequence alteration or mutation and screening for antigen targeting with desired characteristics. Examples of desired characteristics include but are not limited to: increased binding affinity for antigen relative to known antibodies which are specific for the antigen; increased neutralisation of an antigen activity relative to known antibodies which are specific for the antigen if the activity is known; specified competitive ability with a known antibody or ligand to the antigen at a specific molar ratio; ability to immunoprecipitate ligand-receptor complex; ability to bind to a specified epitope; linear epitope, e.g. peptide sequence identified using peptide-binding scan, e.g. using peptides screened in linear and/or constrained conformation; conformational epitope, formed by non-continuous residues; ability to modulate a new biological activity of heparanase, or downstream molecule; ability to bind and/or neutralise heparanase and/or for any other desired property. The techniques required to make substitutions within amino acid sequences of CDRs, antibody VH or VL domains and antigen binding sites are available in the art. Variants of antibody molecules disclosed herein may be produced and used in the present invention. Following the lead of computational chemistry in applying multivariate data analysis techniques to the structure/property-activity relationships (Wold, et al. Multivariate data analysis in chemistry. Chemometrics—Mathematics and Statistics in Chemistry (Ed.: B. Kowalski), D. Reidel Publishing Company, Dordrecht, Holland, 1984) quantitative activity-property relationships of antibodies can be derived using well-known mathematical techniques, such as statistical regression, pattern recognition and classification (Norman et al. Applied Regression Analysis. Wiley-Interscience; 3rd edition (April 1998); Kandel, Abraham & Backer, Eric. Computer-Assisted Reasoning in Cluster Analysis. Prentice Hall PTR, (May 11, 1995); Krzanowski, Wojtek. Principles of Multivariate Analysis: A User's Perspective (Oxford Statistical Science Series, No 22 (Paper)). Oxford University Press; (December 2000); Witten, Ian H. & Frank, Eibe. Data Mining: Practical Machine Learning Tools and Techniques with Java Implementations. Morgan Kaufmann; (Oct. 11, 1999);Denison David G. T. (Editor), Christopher C. Holmes, Bani K. Mallick, Adrian F. M. Smith. Bayesian Methods for Nonlinear Classification and Regression (Wiley Series in Probability and Statistics). John Wiley & Sons; (July 2002); Ghose, Arup K. & Viswanadhan, Vellarkad N. Combinatorial Library Design and Evaluation Principles, Software, Tools, and Applications in Drug Discovery). In some cases the properties of antibodies can be derived from empirical and theoretical models (for example, analysis of likely contact residues or calculated physicochemical property) of antibody sequence, functional and three-dimensional structures and these properties can be considered singly and in combination. An antibody antigen-binding site composed of a VH domain and a VL domain is typically formed by six loops of polypeptide: three from the light chain variable domain (VL) and three from the heavy chain variable domain (VH). Analysis of antibodies of known atomic structure has elucidated relationships between the sequence and three-dimensional structure of antibody combining sites. These relationships imply that, except for the third region (loop) in VH domains, binding site loops have one of a small number of main-chain conformations: canonical structures. The canonical structure formed in a particular loop has been shown to be determined by its size and the presence of certain residues at key sites in both the loop and in framework regions.

This study of sequence-structure relationship can be used for prediction of those residues in an antibody of known sequence, but of an unknown three-dimensional structure, which are important in maintaining the three-dimensional structure of its CDR loops and hence maintain binding specificity. These predictions can be backed up by comparison of the predictions to the output from lead optimisation experiments. In a structural approach, a model can be created of the antibody molecule using any freely available or commercial package, such as WAM. A protein visualisation and analysis software package, such as Insight II (Accelrys, Inc.) or Deep View may then be used to evaluate possible substitutions at each position in the CDR. This information may then be used to make substitutions likely to have a minimal or beneficial effect on activity or confer other desirable properties.

The term “polypeptide fragment” as used herein refers to a polypeptide that has an is amino-terminal and/or carboxy-terminal deletion, but where the remaining amino acid sequence is identical to the corresponding positions in the naturally occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to heparanase, under suitable binding conditions, (2) ability to block appropriate heparanase-substrate binding, or (3) ability to inhibit heparanase activity. Typically, polypeptide analogs comprise a conservative amino acid substitution (or addition or deletion) with respect to the naturally occurring sequence. Analogs typically are at least 20 amino acids long, preferably at least 50 amino acids long or longer, and can often be as long as a full-length naturally occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerised molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH—, —CH═CH-(cis and trans), —COCH₂—, —CH(OH)CH₂—, and —CH₂SO—, by methods well known in the art. Systematic substitution of one or more amino to acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclise the peptide.

As used herein “antibody” and “antibodies”, also known as immunoglobulins, may be an oligoclonal antibody, a polyclonal antibody, a monoclonal antibody (including full-length monoclonal antibodies), a camelised antibody, a chimeric antibody, a CDR-grafted antibody, a multi-specific antibody formed from at least two different epitope binding fragments, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species including, but not limited to, humans, monkeys, pigs, horses, rabbits, dogs, cats, mice, etc., or other animals such as birds (e.g. chickens).

An antibody comprises a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. The variable regions of each light/heavy chain pair form an antibody binding site. Such antibodies may be derived from any mammal, including, but not limited to, humans, monkeys, pigs, horses, rabbits, dogs, cats, mice, etc.

The term “antibody” or “antibodies” includes binding fragments of the antibodies of the invention, exemplary fragments include single-chain Fvs (scFv), single-chain antibodies, single domain antibodies, domain antibodies, Fv fragments, Fab fragments, F(ab′) fragments, F(ab′)2 fragments, antibody fragments that exhibit the desired biological activity, disulfide-stabilised variable region (dsFv), dimeric variable region (Diabody), anti-idiotypic (anti-Id) antibodies (including, e.g., anti-Id antibodies to antibodies of the invention), intrabodies, linear antibodies, single-chain antibody molecules and multispecific antibodies formed from antibody fragments and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2), subclass, or allotype (e.g., Gm, e.g., G1m(f, z, a or x), G2m(n), G3m(g, b, or c), Am, Em, and Km(1, 2 or 3)).

Digestion of antibodies with the enzyme, papain, results in two identical antigen-binding fragments, known also as “Fab” fragments, and a “Fe” fragment, having no antigen-binding activity but having the ability to crystallise. Digestion of antibodies with the enzyme, pepsin, results in the a F(ab)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” when used herein refers to a fragment of an antibody that comprises the constant domain of the light chain and the CH1 domain of the heavy chain.

“dAb” when used herein refers to a fragment of an antibody that is the smallest functional binding unit of a human antibodies. A “dAb” is a single domain antibody and comprises either the variable domain of an antibody heavy chain (VH domain) or the variable domain of an antibody light chain (VL domain). Each dAb contains three of the six naturally occurring CDRs (Ward et al., Binding activities of a repertoire of single immunoglobulin variable domains secreted from Escherichia coli. Nature 341, 544-546 (1989); Holt, et al., Domain antibodies: protein for therapy, Trends Biotechnol. 21, 484-49 (2003)). With molecular weights ranging from 11 to 15 kDa, they are four times smaller than a fragment antigen binding (Fab)2 and half the size of a single chain Fv (scFv) molecule.

“Camelid” when used herein refers to antibody molecules are composed of heavy-chain dimers which are devoid of light chains, but nevertheless have an extensive antigen-binding repertoire (Hamers-Casterman C, Atarhouch T, Muyldermans S, Robinson G, Hamers C, Songa E B, Bendahman N, Hamers R (1993) Naturally occurring antibodies devoid of light chains. Nature 363:446-448).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

It has been shown that fragments of a whole antibody can perform the function of binding antigens. Examples of binding fragments are (Ward, E. S. et al., (1989) Nature 341, 544-546) the Fab fragment consisting of VL, VH, CL and CH1 domains; (McCafferty et al (1990) Nature, 348, 552-554) the Fd fragment consisting of the VH and CH1 domains; (Holt et al (2003) Trends in Biotechnology 21, 484-490) the Fv fragment consisting of the VL and VH domains of a single antibody; (iv) the dAb fragment (Ward, E. S. et al., Nature 341, 544-546 (1989), McCafferty et al (1990) Nature, 348, 552-554, Holt et al (2003) Trends in Biotechnology 21, 484-490], which consists of a VH or a VL domain; (v) isolated CDR regions; (vi) F(ab')2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, (1988) Science, 242, 423-426, Huston et al, (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger, P. (1993) et al, Proc. Natl. Acad. Sci. USA 90 6444-6448,). Fv, scFv or diabody molecules may be stabilised by the incorporation of disulphide bridges linking the VH and VL domains (Reiter, Y. et al, Nature Biotech, 14, 1239-1245, 1996). Minibodies comprising a scFv joined to a CH3 domain may also be made (Hu, S. et al, (1996) Cancer Res., 56, 3055-3061). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab' fragment in which the cysteine residue(s) of the constant domains bear a free thiol group.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “hypervariable region” when used herein refers to the amino acid residues of an antibody which are associated with its binding to antigen. The hypervariable regions encompass the amino acid residues of the “complementarity determining regions” or “CDRs” (e.g., residues 24-34 (L1), 50-56 (L2) and 89-97 (L3) of the light chain variable domain and residues 31-35 (H1), 50-65 (H2) and 95-102(H3) of the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol., 196:901-917 (1987)).

“Framework” or “FR” residues are those variable domain residues flanking the CDRs. FR residues are present in chimeric, humanized, human, domain antibodies, diabodies, vaccibodies, linear antibodies, and bispecific antibodies.

As used herein, “targeted binding agent”, “targeted binding protein”, “specific binding protein” and like terms refer to an agent, for example an antibody, or binding fragment thereof, that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope. A targeted binding agent may comprise at least one antigen binding domain (e.g. a CDR) of an antibody, wherein said domain is fused or contained within a heterologous protein scaffold, e.g. a non-antibody protein scaffold.

“Binding fragments” of an antibody are produced by recombinant DNA techniques, or by enzymatic or chemical cleavage of intact antibodies. Binding fragments include Fab, Fab′, F(ab′)₂, Fv, dAb and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

“Active” or “activity” in regard to a heparanase polypeptide refers to a portion of an heparanase polypeptide that has a biological or an immunological activity of a native heparanase polypeptide. “Biological” when used herein refers to a biological function that results from the activity of the native heparanase polypeptide. A preferred heparanase biological activity includes, for example, heparanase induced cell proliferation, cell adhesion, angiogenesis and invasion.

“Mammal” when used herein refers to any animal that is considered a mammal.

Preferably, the mammal is human.

“Animal” when used herein encompasses animals considered a mammal.

Preferably the animal is human.

The term “patient” includes human and veterinary subjects.

The term “mAb” refers to monoclonal antibody.

“Liposome” when used herein refers to a small vesicle that may be useful for delivery of drugs that may include the heparanase polypeptide of the invention or antibodies to such an heparanase polypeptide to a mammal.

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase.

Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes; additional fluorescent labels or fluorescers include, such as fluorescein and its derivatives, fluorochrome, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates e.g. Europium etc (Perkin Elmer and Cis Biointernational); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), (incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, monocytes, neutrophils, and macrophages) recognise bound antibody on a target cell and subsequently cause lysis of the target cell. The primary cells for mediating ADCC, NK cells, express FcγRIII only, whereas monocytes express FcγRI, FcγRII and FcγRIII. FcRs expression on hematopoietic cells is summarised in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362, or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. PNAS (USA) 95:652-656 (1988).

“Complement dependent cytotoxicity” and “CDC” refer to the mechanism by which antibodies carry out their cell-killing function. It is initiated by the binding of C1q, a constituent of the first component of complement, to the Fc domain of Igs, IgG or IgM, which are in complex with antigen (Hughs-Jones, N. C., and B. Gardner. 1979. Mol. Immunol. 16:697). C1q is a large, structurally complex glycoprotein of ˜410 kDa present in human serum at a concentration of 70 μg/ml (Cooper, N. R. 1985. Adv. Immunol. 37:151). Together with two serine proteases, C1r and C1s, C1q forms the complex C1, the first component of complement. At least two of the N-terminal globular heads of C1q must be bound to the Fc of Igs for C1 activation, hence for initiation of the complement cascade (Cooper, N. R. 1985. Adv. Immunol. 37:151).

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called a, 6, a, y, and respectively. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate in humans. Human light chain constant regions may be classified into two major classes, kappa and lambda.

If desired, the isotype of an antibody that specifically binds heparanase can be switched, for example to take advantage of a biological property of a different isotype. For example, in some circumstances it can be desirable in connection with the generation of antibodies as therapeutic antibodies against heparanase that the antibodies be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgA, human IgG1, and human IgG3. In other embodiments it can be desirable in connection with the generation of antibodies as therapeutic antibodies against heparanase that the antibodies be capable of binding Fe receptors on effector cells and participating in antibody-dependent cytotoxicity (ADCC). There are a number of isotypes of antibodies that are capable of the same, including, without limitation, the following: murine IgG2a, murine IgG2b, murine IgG3, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

By way of example, the anti-heparanase antibodies discussed herein are fully human antibodies. If an antibody possessed desired binding to heparanase, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC and/or be capable of binding to Fc receptors on effector cells and participating in ADCC.

“Whole blood assays” use unfractionated blood as a source of natural effectors. Blood contains complement in the plasma, together with FcR-expressing cellular effectors, such as polymorphonuclear cells (PMNs) and mononuclear cells (MNCs). Thus, whole blood assays allow simultaneous evaluation of the synergy of both ADCC and CDC effector mechanisms in vitro.

A “therapeutically effective” amount as used herein is an amount that provides some improvement or benefit to the subject. Stated in another way, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

Exemplary cancers in humans include a bladder tumour, breast tumour, prostate tumour, basal cell carcinoma, biliary tract cancer, bladder cancer, bone cancer, brain and CNS cancer (e.g., glioma tumour), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas.

The term “and/or” as used herein is to be taken as specific disclosure of each of the two specified features or components with or without the other. For example “A and/or B” is to be taken as specific disclosure of each of (i) A, (ii) B and (iii) A and B, just as if each were set out individually herein.

### Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Bispecific antibodies do not exist in the form of fragments having a single binding site (e.g., Fab, Fab′, and Fv).

Typically, a VH domain is paired with a VL domain to provide an antibody antigen-binding site, although a VH or VL domain alone may be used to bind antigen. The VH domain (see Table 5) may be paired with the VL domain (see Table 6), so that an antibody antigen-binding site is formed comprising both the VH and VL domains.

### Human Antibodies and Humanisation of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilisation of murine or rat derived antibodies, fully human antibodies can be generated through the introduction of functional human antibody loci into a rodent, other mammal or animal so that the rodent, other mammal or animal produces fully human antibodies.

One method for generating fully human antibodies is through the use of XenoMouse® strains of mice that have been engineered to contain up to but less than 1000 kb-sised germline configured fragments of the human heavy chain locus and kappa light chain locus. See Mendez et al. Nature Genetics 15:146-156 (1997) and Green and Jakobovits J. Exp. Med. 188:483-495 (1998). The XenoMouse® strains are available from Amgen, Inc. (Fremont, Calif., U.S.A).

Such mice, then, are capable of producing human immunoglobulin molecules and antibodies and are deficient in the production of murine immunoglobulin molecules and antibodies. Technologies utilised for achieving the same are disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

The production of the XenoMouse® strains of mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, Ser. No. 08/031,801, filed Mar. 15, 1993, Ser. No. 08/112,848, filed Aug. 27, 1993, Ser. No. 08/234,145, filed April 28, 1994, Ser. No. 08/376,279, filed Jan. 20, 1995, Ser. No. 08/430, 938, filed Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 8/462,513, filed Jun. 5, 1995, Ser. No. 08/724,752, filed Oct. 2, 1996, Ser. No. 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilised a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed is Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference. Additionally, KM™—mice, which are the result of cross-breeding of Kirin's Tc mice with Medarex's minilocus (Humab) mice have been generated. These mice possess the human IgH transchromosome of the Kirin mice and the kappa chain transgene of the Genpharm mice (Ishida et al., Cloning Stem Cells, (2002) 4:91-102).

Human antibodies can also be derived by in vitro methods. Suitable examples include but are not limited to phage display (CAT, Morphosys, Dyax, Biosite/Medarex, Xoma, Symphogen, Alexion (formerly Proliferon), Affimed) ribosome display (CAT), yeast display, and the like.

### Preparation of Antibodies

Antibodies, as described herein, were prepared through the utilization of the XenoMouse® technology, as described below. Such mice are capable of producing human immunoglobulin molecules and antibodies and are deficient in the production of murine immunoglobulin molecules and antibodies. Technologies utilised for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through the use of such technology, fully human monoclonal antibodies to a variety of antigens have been produced. Essentially, XenoMouse® lines of mice are immunised with an antigen of interest (e.g. heparanase), lymphatic cells (such as B-cells) are recovered from the hyper-immunised mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to heparanase. Further, provided herein are characterisation of the antibodies produced by such cell lines, including nucleotide and amino acid sequence analyses of the heavy and light chains of such antibodies.

Alternatively, instead of being fused to myeloma cells to generate hybridomas, B cells can be directly assayed. For example, CD19+ B cells can be isolated from hyperimmune XenoMouse® mice and allowed to proliferate and differentiate into antibody-secreting plasma cells. Antibodies from the cell supernatants are then screened by ELISA for reactivity against the heparanase immunogen. The supernatants might also be screened for immunoreactivity against fragments of heparanase to further map the different antibodies for binding to domains of functional interest on heparanase. The antibodies may also be screened other related human endoglycosidases and against the rat, the mouse, and non-human primate, such as Cynomolgus monkey, orthologues of heparanase, the last to determine species cross-reactivity. B cells from wells containing antibodies of interest may be immortalised by various methods including fusion to make hybridomas either from individual or from pooled wells, or by infection with EBV or transfection by known immortalising genes and then plating in suitable medium. Alternatively, single plasma cells secreting antibodies with the desired specificities are then isolated using a heparanase-specific hemolytic plaque assay (see for example Babcook et al., Proc. Natl. Acad. Sci. USA 93:7843-48 (1996)). Cells targeted for lysis are preferably sheep red blood cells (SRBCs) coated with the heparanase antigen.

In the presence of a B-cell culture containing plasma cells secreting the immunoglobulin of interest and complement, the formation of a plaque indicates specific heparanase-mediated lysis of the sheep red blood cells surrounding the plasma cell of interest. The single antigen-specific plasma cell in the center of the plaque can be isolated and the genetic information that encodes the specificity of the antibody is isolated from the single plasma cell. Using reverse-transcription followed by PCR (RT-PCR), the DNA encoding the heavy and light chain variable regions of the antibody can be cloned. Such cloned DNA⁻can then be further inserted into a suitable expression vector, preferably a vector cassette such as a pcDNA, more preferably such a pcDNA vector containing the constant domains of immunglobulin heavy and light chain. The generated vector can then be transfected into host cells, e.g., HEK293 cells, CHO cells, and cultured in conventional nutrient media modified as appropriate for inducing transcription, selecting transformants, or amplifying the genes encoding the desired sequences.

As will be appreciated, antibodies as described herein can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalised cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells (Hek293), and a number of other cell lines. Cell lines of particular preference are selected through determining which cell lines have high expression levels and produce antibodies with constitutive heparanase binding properties.

In the cell-cell fusion technique, a myeloma, CHO cell or other cell line is prepared that possesses a heavy chain with any desired isotype and another myeloma, CHO cell or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

In the cell-cell fusion technique, a myeloma, CHO cell or other cell line is prepared that possesses a heavy chain with any desired isotype and another myeloma, CHO cell or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

### Antibody Sequences

Embodiments of the invention include the antibodies listed below in Table 1. This table reports the identification number of each antibody, along with the SEQ ID number of the variable domain of the corresponding heavy chain and light chain genes and polypeptides, respectively. Each antibody has been given an identification number.

Each antibody has been given an identification number that includes two numbers separated by one decimal point

TABLE 1

| | | SEQ |
|---|---|---|
| mAb ID | | ID |
| No.: | Sequence | NO: |
| 10E9.1 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 15A1.2 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 15A11.1 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 16G1.1 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 16G1.2 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 7B9.1 | Nucleotide sequence encoding the variable region of the heavy chain | 21 |
| | Amino acid sequence encoding the variable region of the heavy chain | 22 |
| | Nucleotide sequence encoding the variable region of the light chain | 23 |
| | Amino acid sequence encoding the variable region of the light chain | 24 |
| 14H12.3 | Nucleotide sequence encoding the variable region of the heavy chain | 25 |
| | Amino acid sequence encoding the variable region of the heavy chain | 26 |
| | Nucleotide sequence encoding the variable region of the light chain | 27 |
| | Amino acid sequence encoding the variable region of the light chain | 28 |
| 16E1.1 | Nucleotide sequence encoding the variable region of the heavy chain | 29 |
| | Amino acid sequence encoding the variable region of the heavy chain | 30 |
| | Nucleotide sequence encoding the variable region of the light chain | 31 |
| | Amino acid sequence encoding the variable region of the light chain | 32 |

### Therapeutic Administration and Formulations

Embodiments of the invention include sterile pharmaceutical formulations of anti-heparanase antibodies that are useful as treatments for diseases. Such formulations would inhibit the binding of heparanase to its substrates, thereby treating pathological conditions where, for example, serum or tissue heparanase is abnormally elevated. Antibodies of the invention preferably possess adequate affinity to potently inhibit heparanase activity, or inhibit heparanase binding to its substrates, and preferably have an adequate duration of action to allow for infrequent dosing in humans. A prolonged duration of action will allow for less frequent and more convenient dosing schedules by alternate parenteral routes such as subcutaneous or intramuscular injection.

Sterile formulations can be created, for example, by filtration through sterile filtration membranes, prior to or following lyophilisation and reconstitution of the antibody. The antibody ordinarily will be stored in lyophilised form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, direct injection to a tumour site, or by sustained release systems as noted below. The antibody is preferably administered continuously by infusion or by bolus injection.

An effective amount of antibody to be employed therapeutically will depend, for example, upon the therapeutic objectives, the route of administration, and the condition of the patient. Accordingly, it is preferred that the therapist titer the dosage and modify the route of administration as required to obtain the optimal therapeutic effect. Typically, the clinician will administer antibody until a dosage is reached that achieves the desired effect. The progress of this therapy is easily monitored by conventional assays or by the assays described herein.

Antibodies, as described herein, can be prepared in a mixture with a pharmaceutically acceptable carrier. This therapeutic composition can be administered intravenously or through the nose or lung, preferably as a liquid or powder aerosol (lyophilised). The composition can also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with pharmaceutically acceptable carriers, excipients, or stabilisers. Such materials are non-toxic to the recipients at the dosages and concentrations employed, and include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they can denature or aggregate as a result of exposure to moisture at 37° C., resulting in a loss of biological activity and possible changes in immunogenicity. Rational strategies can be devised for protein stabilisation depending on the mechanism involved. For example, if the aggregation mechanism is discovered to be intermolecular S—S bond formation through disulfide interchange, stabilisation can be achieved by modifying sulfhydryl residues, lyophilising from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient will be determined by the attending physician taking into consideration various factors known to modify the action of drugs including severity and type of disease, body weight, sex, diet, time and route of administration, other medications and other relevant clinical factors. Therapeutically effective dosages can be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.0001 mg/kg, 0.001 mg/kg, 0.01 mg/kg, 0.1 mg/kg, 1 mg/kg, 10 mg/kg to up to 100 mg/kg, 1000 mg/kg, 10000 mg/kg or more, of the patient's body weight depending on the factors mentioned above. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight depending on the factors mentioned above. Typically, the clinician will administer the therapeutic antibody until a dosage is reached that achieves the desired effect. The progress of this therapy is easily monitored by conventional assays or as described herein.

Doses of antibodies of the invention may be repeated and the administrations may be separated by at least 1 day, 2 days, 3 days, 5 days, 10 days, 15 days, 30 days, 45 days, 2 months, 75 days, 3 months, or at least 6 months.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures can be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regal. Toxicol. Pharmacol. 32(2):210-8 (2000), Wang W. “Lyophilisation and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Design and Generation of Other Therapeutics

In accordance with the present invention and based on the activity of the antibodies that are produced and characterised herein with respect to heparanase, the design of other therapeutic modalities beyond antibody moieties is facilitated and disclosed to one of skill in the art. Such modalities include, without limitation, advanced antibody therapeutics, such as bispecific antibodies, immunotoxins, radiolabeled therapeutics, and single antibody V domains, antibody-like binding agent based on other than V region scaffolds, single domain antibodies, generation of peptide therapeutics, heparanase binding domains in novel scaffolds, gene therapies, particularly intrabodies, antisense therapeutics, and small molecules.

An antigen binding site may be provided by means of arrangement of CDRs on non-antibody protein scaffolds, such as fibronectin or cytochrome B etc. (Haan & Maggos (2004) BioCentury, 12(5): A1-A6; Koide et al. (1998) Journal of Molecular Biology, 284: 1141-1151; Nygren et al. (1997) Current Opinion in Structural Biology, 7: 463-469) or by randomising or mutating amino acid residues of a loop within a protein scaffold to confer binding specificity for a desired target. Scaffolds for engineering novel binding sites in proteins have been reviewed in detail by Nygren et al. (Nygren et al. (1997) Current Opinion in Structural Biology, 7: 463-469). Protein scaffolds for antibody mimics are disclosed in WO/0034784, which is herein incorporated by reference in its entirety, in which the inventors describe proteins (antibody mimics) that include a fibronectin type III domain having at least one randomised loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of a binding member may confer useful physiological properties, such as an ability to enter cells, penetrate deep into tissues or reach targets within other structures, or to bind within protein cavities of the target antigen. Use of antigen binding sites in non-antibody protein scaffolds is reviewed in Wess, 2004 (Wess, L. In: BioCentury, The Bernstein Report on BioBusiness, 12(42), A1-A7, 2004). Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, albumin, tetranectin, fibronectin (e.g. 10th fibronectin type III domain), lipocalins as well as gamma-crystalline and other Affilin™ scaffolds (Soil Proteins). Examples of other approaches include synthetic “Microbodies” based on cyclotides—small proteins having intra-molecular disulphide bonds, Microproteins (Versabodies™, Amunix) and ankyrin repeat proteins (DARPins, Molecular Partners).

In addition to antibody sequences and/or an antigen-binding site, a targeted binding agent according to the present invention may comprise other amino acids, e.g. forming a peptide or polypeptide, such as a folded domain, or to impart to the molecule another functional characteristic in addition to ability to bind antigen. Targeted binding agents of the invention may carry a detectable label, or may be conjugated to a toxin or a targeting moiety or enzyme (e.g. via a peptidyl bond or linker). For example, a targeted binding agent may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

In connection with the generation of advanced antibody therapeutics, where complement fixation is a desirable attribute, it can be possible to sidestep the dependence on complement for cell killing through the use of bispecific antibodies, immunotoxins, or radiolabels, for example.

For example, bispecific antibodies can be generated that comprise (i) two antibodies one with a specificity to heparanase and another to a second molecule that are conjugated together, (ii) a single antibody that has one chain specific to heparanase and a second chain specific to a second molecule, or (iii) a single chain antibody that has specificity to heparanase and the other molecule. Such bispecific antibodies can be generated using techniques that are well known; for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made as desired. For example, the second specificity can be made to the heavy chain activation receptors, including, without limitation, CD16 or CD64 (see e.g., Deo et al. Immunol. Today 18:127 (1997)) or CD89 (see e.g., Valerius et al. Blood 90:4485-4492 (1997)).

Antibodies can also be modified to act as immunotoxins utilising techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilising techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each immunotoxin or radiolabeled molecule would be likely to kill cells expressing the desired multimeric enzyme subunit oligomerisation domain.

When an antibody is linked to an agent (e.g., radioisotope, pharmaceutical composition, or a toxin) it is contemplated that the agent possesses a pharmaceutical property selected from the group of antimitotic, alkylating, antimetabolite, antiangiogenic, apoptotic, alkaloid, COX-2, and antibiotic agents and combinations thereof. The agent can be selected from the group of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antimetabolites, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, oxaliplatin, doxorubicins and their analogs, and a combination thereof. Examples of toxins further include gelonin, Pseudomonas exotoxin (PE), PE40, PE38, diphtheria toxin, ricin, abrin, alpha toxin, saporin, ribonuclease (RNase), DNase I, Staphylococcal enterotoxin-A, pokeweed antiviral protein, gelonin, Pseudomonas endotoxin, members of the enediyne family of molecules, such as calicheamicin and esperamicin, as well as derivatives, combinations and modifications thereof. Chemical toxins can also be taken from the group consisting of duocarmycin (see, e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Examples of chemotherapeutic agents also include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see, U.S. Pat. No. 4,675,187), Melphalan, and other related nitrogen mustards. Suitable toxins and chemotherapeutic agents are described in Remington's Pharmaceutical Sciences, 19th Ed. (Mack Publishing Co. 1995), and in Goodman And Gilman's The Pharmacological Basis of Therapeutics, 7th Ed. (MacMillan Publishing Co. 1985). Other suitable toxins and/or chemotherapeutic agents are known to those of skill in the art.

Examples of radioisotopes include gamma-emitters, positron-emitters, and x-ray emitters that can be used for localisation and/or therapy, and beta-emitters and alpha-emitters that can be used for therapy. The radioisotopes described previously as useful for diagnostics, prognostics and staging are also useful for therapeutics.

Non-limiting examples of anti-cancer or anti-leukemia agents include anthracyclines such as doxorubicin (adriamycin), daunorubicin (daunomycin), idarubicin, detorubicin, carminomycin, epirubicin, esorubicin, and morpholino and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. Preferably, the anti-cancer or anti-leukemia is doxorubicin, morpholinodoxorubicin, or morpholinodaunorubicin.

The antibodies of the invention also encompass antibodies that have half-lives (e.g., serum half-lives) in a mammal, preferably a human, of greater than that of an unmodified antibody. In one embodiment, said antibody half life is greater than about 15 days, greater than about 20 days, greater than about 25 days, greater than about 30 days, greater than about 35 days, greater than about 40 days, greater than about 45 days, greater than about 2 months, greater than about 3 months, greater than about 4 months, or greater than about 5 months. The increased half-lives of the antibodies of the present invention or fragments thereof in a mammal, preferably a human, result in a higher serum titer of said antibodies or antibody fragments in the mammal, and thus, reduce the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication Nos. WO 97/34631 and WO 02/060919, which are incorporated herein by reference in their entireties). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatisation that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

As will be appreciated by one of skill in the art, in the above embodiments, while affinity values can be important, other factors can be as important or more so, depending upon the particular function of the antibody. For example, for an immunotoxin (toxin associated with an antibody), the act of binding of the antibody to the target can be useful; however, in some embodiments, it is the internalisation of the toxin into the cell that is the desired end result. As such, antibodies with a high percent internalisation can be desirable in these situations. Thus, in one embodiment, antibodies with a high efficiency in internalisation are contemplated. A high efficiency of internalisation can be measured as a percent internalised antibody, and can be from a low value to 100%. For example, in varying embodiments, 0.1-5, 5-10, 10-20, 20-30, 30-40, 40-45, 45-50, 50-60, 60-70, 70-80, 80-90, 90-99, and 99-100% can be a high efficiency. As will be appreciated by one of skill in the art, the desirable efficiency can be different in different embodiments, depending upon, for example, the associated agent, the amount of antibody that can be administered to an area, the side effects of the antibody-agent complex, the type (e.g., cancer type) and severity of the problem to be treated.

In other embodiments, the antibodies disclosed herein provide an assay kit for the detection of heparanase expression in mammalian tissues or cells in order to screen for a disease or disorder associated with changes in expression of heparanase. The kit comprises an antibody that binds heparanase and means for indicating the reaction of the antibody with the antigen, if present.

In some embodiments, an article of manufacture is provided comprising a container, comprising a composition containing an antibody that specifically binds heparanase, and a package insert or label indicating that the composition can be used to treat disease mediated by heparanase expression. Preferably a mammal and, more preferably, a human, receives the antibody that specifically binds heparanase.

### Combinations

The anti-tumour treatment defined herein may be applied as a sole therapy or may involve, in addition to the compounds of the invention, conventional surgery, bone marrow and peripheral stem cell transplantations or radiotherapy or chemotherapy. Such chemotherapy may include one or more of the following categories of anti tumour agents:
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or inhibitors of cathepsin activity, inhibitors of serine proteases for example matriptase, hepsin, urokinase and inhibitors of integrin αvβ6 function.
(iv) cytotoxic agents such as fludarabine, 2-chlorodeoxyadenosine, chlorambucil or doxorubicin and combination thereoff such as Fludarabine+cyclophosphamide, CVP: cyclophosphamide+vincristine+prednisone, ACVBP: doxorubicin+cyclophosphamide+vindesine+bleomycin+prednisone, CHOP: cyclophosphamide+doxorubicin+vincristine+prednisone, CNOP: cyclophosphamide+mitoxantrone+vincristine+prednisone, m-BACOD: methotrexate+bleomycin+doxorubicin+cyclophosphamide+vincristine+dexamethasone+leucovorin, MACOP-B: methotrexate+doxorubicin+cyclophosphamide+vincristine+prednisone fixed dose+bleomycin+leucovorin, or ProMACE CytaBOM: prednisone+doxorubicin+cyclophosphamide+etoposide+cytarabine+bleomycin+vincristine+methotrexate+leucovorin.
(v) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors, and inhibitors of survival signaling proteins such as Bcl-2, Bcl-XL for example ABT-737;
(vi) antiangiogenic agents such as those which inhibit the effects of vascular endothelial growth factor, [for example the anti-vascular endothelial cell growth factor antibody bevacizumab (Avastin™)] and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856, WO 98/13354, WO00/47212 and WO01/32651,anti-vascular endothelial growth factor receptor antibodies such anti-KDR antibodies and anti-flt1 antibodies,) and compounds that work by other mechanisms (for example linomide, inhibitors of integrin αvβ3 function and angiostatin)] or colony stimulating factor 1 (CSF1) or CSF1 receptor; Additional details on AZD2171 may be found in Wedge et al (2005) Cancer Research. 65(10):4389-400. Additional details on AZD6474 may be found in Ryan & Wedge (2005) British Journal of Cancer. 92 Suppl 1:S6-13. Both publications are herein incorporated by reference in their entireties.
(vii) vascular damaging agents such as Combretastatin A4 and compounds disclosed in International Patent Applications WO 99/02166, WO 00/40529, WO 00/41669, WO 01/92224, WO 02/04434 and WO 02/08213;
(viii) antisense therapies, for example those which are directed to the targets listed above, such as ISIS 2503, an anti-ras antisense or G3139 (Genasense), an anti bcl2 antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(x) immunotherapy approaches, including for example treatment with Alemtuzumab (campath-1H™), a monoclonal antibody directed at CD52, or treatment with antibodies directed at CD22, ex vivo and in vivo approaches to increase the immunogenicity of patient tumour cells, transfection with cytokines such as interleukin 2, interleukin 4 or granulocyte macrophage colony stimulating factor, approaches to decrease T cell anergy such as treatment with monoclonal antibodies inhibiting CTLA-4 function, approaches using transfected immune cells such as cytokine transfected dendritic cells, approaches using cytokine transfected tumour cell lines and approaches using anti idiotypic antibodies;
(xi) inhibitors of protein degradation such as proteasome inhibitor such as Velcade (bortezomid); and
(xii) biotherapeutic therapeutic approaches for example those which use peptides or proteins (such as antibodies or soluble external receptor domain constructions) which either sequester receptor ligands, block ligand binding to receptor or decrease receptor signalling (e.g. due to enhanced receptor degradation or lowered expression levels).

In one embodiment the anti-tumour treatment defined herein may involve, in addition to the compounds of the invention, treatment with other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin).

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically active agent within its approved dosage range.

### EXAMPLES

The following examples, including the experiments conducted and results achieved are provided for illustrative purposes only and are not to be construed as limiting upon the teachings herein.

### Example 1

### Expression of Biochemically Active Recombinant Human Heparanse

The expression of active human heparanase was described by McKenzie et al (Biochem J 373 423-425 2003). Heparanase 8 and 50 kDa sub-units were cloned into pacGP67 as Sma 1-EcoR1 fragments and subsequently cloned into the baculovirus transfer vector, pAcUW51 as Bgl II and Eco RI fragments respectively. The cDNAs were then cloned into the Kpn I-Xho I (8kDa) and Eco RI (50 kDa) sites of pFastBac Dual. Heparanase was purified from Hi5 cells, MOI=2, after 48 hrs by elution from a ‘Heparin FF’ column in 0.7M NaCl, 25 mM tris pH7.5; yields were approximately 0.5-1 mg per ml with low endotoxin (˜90EU per ml).

### Example 2

### Immunisation and Titering

### Immunisation

Immunisations were conducted using soluble recombinant human heparanase. Ten μg/mouse soluble protein for Campaign 1, or 25 μg/mouse for Campaign 2, were used for immunisation in XenoMouse™ according to the methods disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. The immunisation programs are summarised in Table 2.

### Selection of Animals for Harvest by Titer

Titres of antibodies from Campaigns 1 and 2 were determined in an ELISA. Costar 3368 medium binding plates were coated directly with recombinant human heparanase or plates were coated with Rabbit anti-heparanase antibody (Raybiotech, Cat. IP-14-155) to capture recombinant heparanase. Directly coated heparanase was detected in three separate plates by mouse (InSight Cat. INS-26-1-0000), goat (Santa Cruz, Cat. sc26136) or rabbit (Raybiotech, Cat. IP-14-155) anti-heparanase antibodies, and quantified using appropriate secondary antibodies. Antibody captured heparanase was detected using mouse anti-heparanase (Insight Cat. INS-26-1-0000). At the end of the immunisation program, fusions were performed using mouse myeloma cells and lymphocytes isolated from the spleens and lymph nodes of the immunised mice by means of electroporation, as described in Example 2.

TABLE 2
Summary of Immunisation Programs

| | | | | No of | Immunisation |
|---|---|---|---|---|---|
| Campaign | Group | Immunogen | Strain | mice | routes |
| 1 | 1 | Soluble | IgG2 | 5 | Fp, twice/wk, ×4 wks |
| | | recombinant | | | |
| | | human | | | |
| | | heparanase | | | |
| 2 | 2 | Soluble | IgG2 | 10 | Fp, twice/wk, ×4 wks |
| | | recombinant | | | |
| | | human | | | |
| | | heparanase | | | |
| 2 | 3 | Soluble | IgG4 | 10 | Fp, twice/wk, ×4 wks |
| | | recombinant | | | |
| | | human | | | |
| | | heparanase | | | |

“Fp” refers to “foot pad”

### Example 3

### Recovery of Lymphocytes, B-Cell Isolations, Fusions and Generation of Hybridomas

Immunised mice were sacrificed by cervical dislocation, and the draining lymph nodes harvested and pooled from each cohort. The lymphoid cells were dissociated by grinding in DMEM to release the cells from the tissues and the cells were suspended in DMEM. The cells were counted, and 0.9 ml DMEM per 100 million lymphocytes added to the cell pellet to resuspend the cells gently but completely. Using 100 μl of CD90+ magnetic beads per 100 million cells, the cells were labeled by incubating the cells with the magnetic beads at 4° C. for 15 minutes. The magnetically labeled cell suspension containing up to 10⁸ positive cells (or up to 2×10⁹ total cells) was loaded onto a LS+ column and the column washed with DMEM. The total effluent was collected as the CD90-negative fraction (most of these cells were expected to be B cells).

The fusion was performed by mixing washed enriched B cells from above and nonsecretory myeloma P3×63Ag8.653 cells purchased from ATCC, cat.# CRL 1580 (Kearney et al, J. Immunol. 123, 1979, 1548-1550) at a ratio of 1:1. The cell mixture was gently pelleted by centrifugation at 800×g. After complete removal of the supernatant, the cells were treated with 2-4 mL of Pronase solution (CalBiochem, cat. # 53702; 0.5 mg/ml in PBS) for no more than 2 minutes. Then 3-5 ml of FBS was added to stop the enzyme activity and the suspension was adjusted to 40 ml total volume using electro cell fusion solution, (ECFS, 0.3M Sucrose, Sigma, Cat#S7903, 0.1 mM Magnesium Acetate, Sigma, Cat#M2545, 0.1 mM Calcium Acetate, Sigma, Cat#C4705). The supernatant was removed after centrifugation and the cells were resuspended in 40 ml ECFS. This wash step was repeated and the cells again were resuspended in ECFS to a concentration of 2×10⁶ cells/ml.

Electro-cell fusion was performed using a fusion generator (model ECM2001, Genetronic, Inc., San Diego, Calif.). The fusion chamber size used was 2.0 ml, using the following instrument settings:

Alignment condition: voltage: 50 V, time: 50 sec.

Membrane breaking at: voltage: 3000 V, time: 30 μsec

Post-fusion holding time: 3 sec

After ECF, the cell suspensions were carefully removed from the fusion chamber under sterile conditions and transferred into a sterile tube containing the same volume of Hybridoma Culture Medium (DMEM, JRH Biosciences), 15% FBS (Hyclone), supplemented with L-glutamine, pen/strep, OPI (oxaloacetate, pyruvate, bovine insulin) (all from Sigma) and IL-6 (Boehringer Mannheim). The cells were incubated for 15-30 minutes at 37° C., and then centrifuged at 400×g (1000 rpm) for five minutes. The cells were gently resuspended in a small volume of Hybridoma Selection Medium (Hybridoma Culture Medium supplemented with 0.5× HA (Sigma, cat. #A9666)), and the volume adjusted appropriately with more Hybridoma Selection Medium, based on a final plating of 5×10⁶B cells total per 96-well plate and 200 μl per well. The cells were mixed gently and pipetted into 96-well plates and allowed to grow. On day 7 or 10, one-half the medium was removed, and the cells re-fed with Hybridoma Selection Medium.

Hybridomas were grown as routine in the selective medium. Exhaustive supernatants collected from the hybridomas that potentially produce anti-human heparanase antibodies were screened for heparanase enzyme inhibitory activity as described in Example 4.

### Example 4

### Heparanase Activity Assay

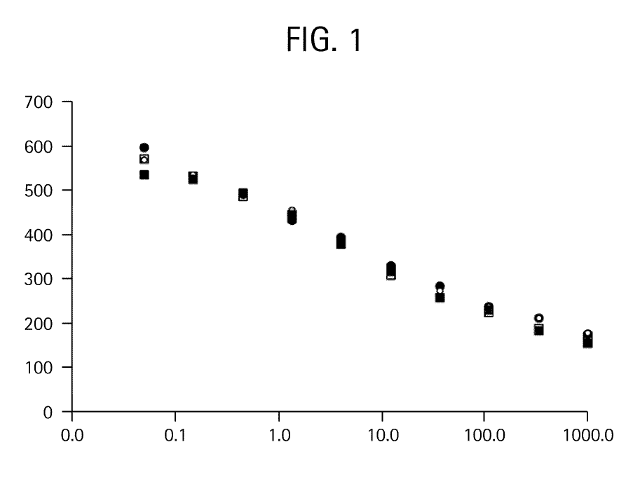

Heparanase activity was determined in a homogeneous time resolved fluorescence (HTRF) heparan sulfate degradation assay using a biotin-heparan sulfate-europium criptate substrate (HS-cryptate, CisBio Cat No. 61 BHSKAA), final concentration 24 nM in 24 μl 200 mM sodium acetate pH5.5. Antibodies were captured from hybridoma culture supernatants onto anti-human IgFc coated Dynabeads to avoid inactivation of recombinant heparanase by hybridoma culture supernatant of pH>5.5. Dynal Dynabeads M280 streptavidin were incubated with biotin rabbit F(ab′)2 anti-human IgG FC (Jackson ImmunoResearch Europe Ltd., Catalogue Number 309-066-008), 250 μg biotin-F(ab′)2 per 10 mg beads, for 1 hr at room temperature with gentle mixing, washed with 3×2ml PBS, and then resuspended at 10 mg beads per ml in PBS for capture of human antibodies from hybridoma supernatants. Incorporating Dynabeads into the heparan sulfate cleavage assay had no effect on heparanase enzyme activity (see FIG. 1).

Biotin anti-human Dynabeads were incubated with hybridoma supernatants at room temperature for 2 hrs with gentle mixing, immobilised with a 96-well plate magnet, and then blocked by incubation with d-biotin in PBS for 1 hr at room temperature; the molar ratio of free biotin to the number of biotin binding sites was 10:1. Immobilised beads were then washed with PBS prior to use in the assay.

Following the removal of excess blocking biotin, the Dynabead-human antibodies were incubated with recombinant heparanase (30 ng per ml, 16 μl per well) and incubated for 1 hr at room temperature and then incubated with 8 μl per well substrate (HS-cryptate) at 37° C. for 1 hr; final concentrations of heparanase and HS-cryptate were 0.2 nM and 24 nM respectively. HS-cryptate degradation was determined by the addition of 15 μl enzyme reaction mixture to 5 μl (50 ng) prediluted Streptavidin-XL665 (CisBio 611SAXLA, final concentration 42 nM). After 30 minutes incubation at room temperature, fluorescence was measured on a Tecan Genesis Pro using 330/60 nm excitation and 655/10 nm emission filters. Enzyme activity was expressed as % ρF=((665/612) sample-(665/612) blank)×100/(665/612) blank. Degradation of the HS-cryptate substrate leads to a low % ρF.

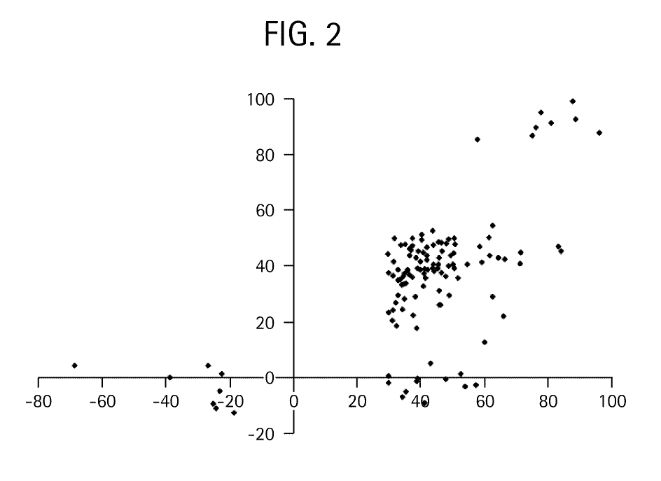

Inhibition of heparanase enzyme activity by hybridoma exhaustive supernatants from repeat experiments is shown in FIG. 2 where the data is plotted as percent inhibition. The majority of hybridoma activities were in the range of 40-60% inhibition with a small number of hybridomas completely inhibiting heparanase activity.

### Example 5

### Cross Species Activity vs Murine and Cynomolgus Heparanase

Initially, cross species binding to human, murine and cynomolgus heparanase was determined in an ELISA assay in which heparanase was captured onto plates using a rabbit anti-heparanase antibody (Raybiotech, Cat. IP-14-155). Exhaustive supernatants were added to the plates at a final dilution of 1:5 and bound antibody detected with a rabbit anti-human IgG Fc. Antibodies exhibiting cross species binding and the desired enzyme inhibition activity were subsequently purified to determine cross species enzyme inhibition.

Human, murine and cynomolgus heparanase were transfected into Hek293 cells and conditioned medium harvested after 7 days culture was filter sterilised, concentrated (10 kDa cut-off spin column) and stored at 4° C. prior to use in the HS-cryptate degradation assay. Purified antibodies were diluted such that final concentrations in the assay were 1000, 100, 10 and 1 ng per ml. Heparanase-Hek293 conditioned media were diluted to final concentrations 1:50, 1:100 and 1:200 in the assay, and the final concentration of HS-cryptate was 24 nM. Suramin, final concentration 7 μM was used as a positive control in the assay to provide a measure of relative activity for the human, murine and cynomolgus enzymes.

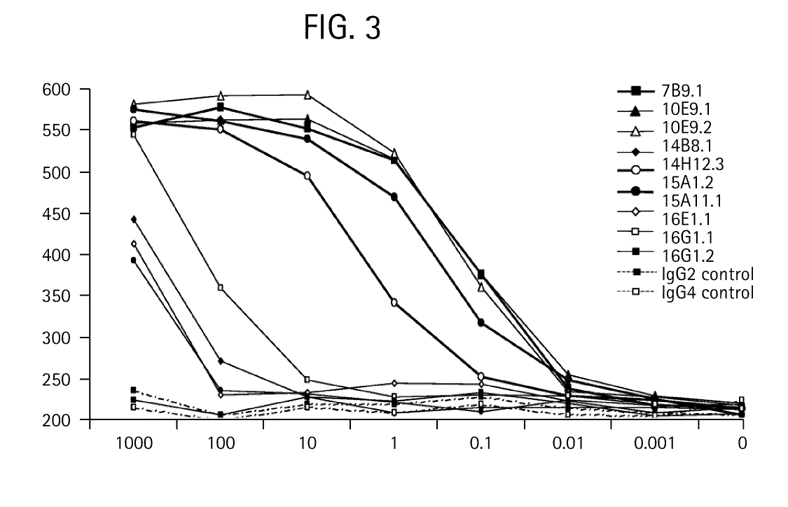

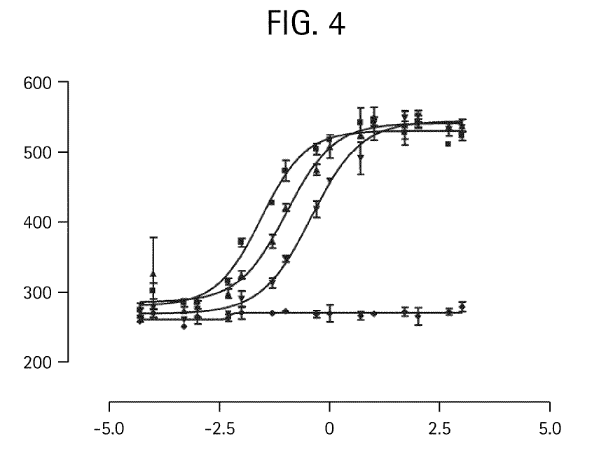

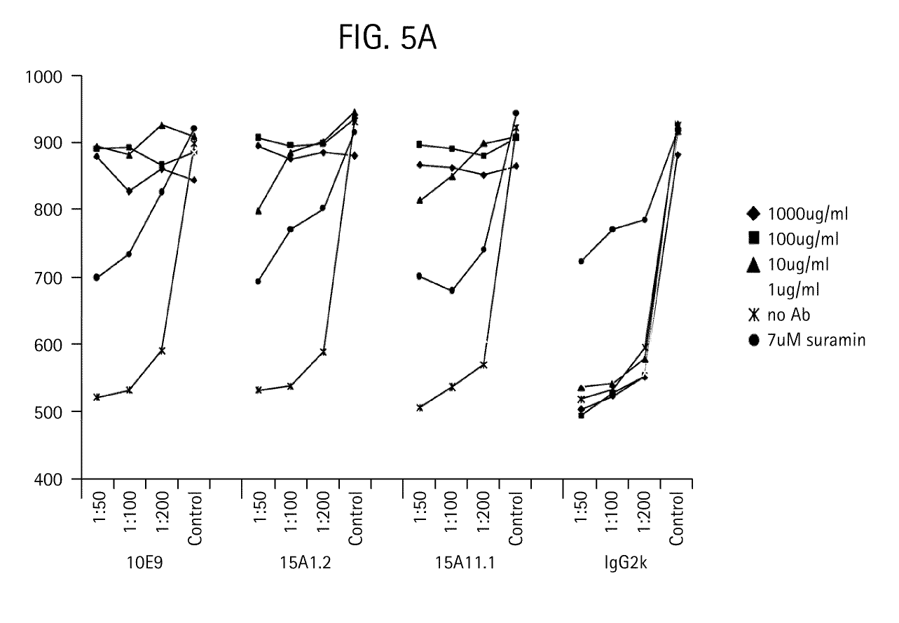

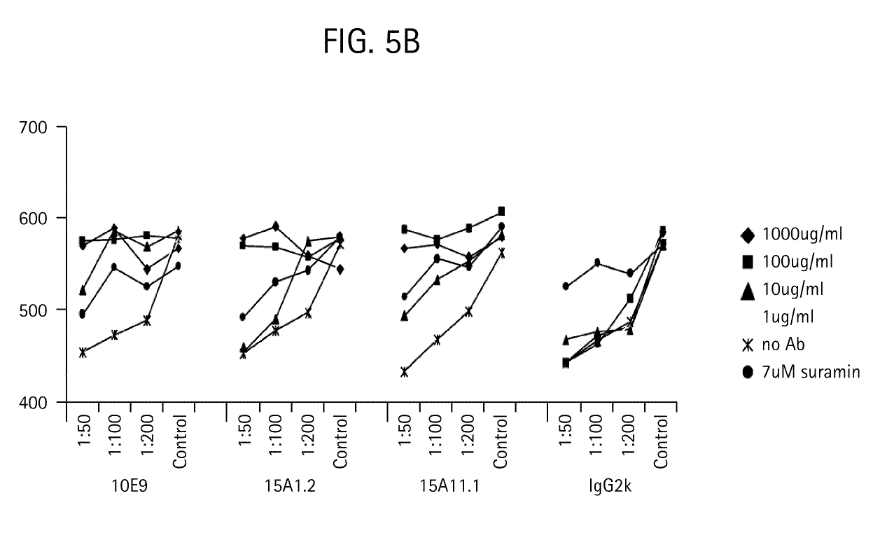

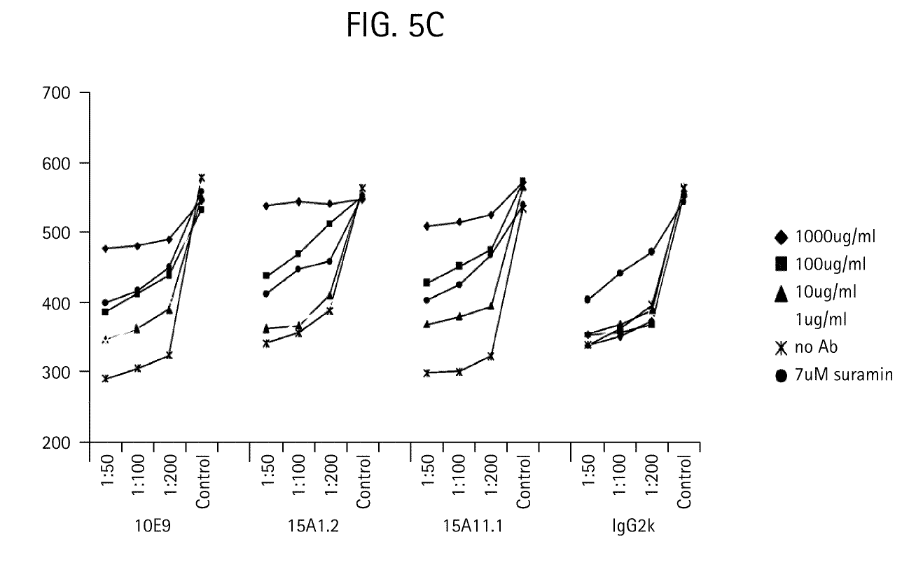

Initial screening to detect antibodies binding to human heparanase in both direct coating and antibody capture ELISAs as described above identified 800 hybridomas for further testing; 535 IgG2 from Campaign 1 and 2 and 265 IgG4 from Campaign 2. Enzyme inhibition assays and cross species binding to cynomolgus and murine heparanase, in an antibody capture ELISA, identifed 12 antibodies that were taken forward for sub-cloning to provide purified antibodies for further analysis. Subsequently, confirmation of activity against human heparanase (FIGS. 3 and 4) and inhibition of recombinant cynomolgus and murine heparanase activity in the HS-cryptate degradation assay (FIG. 5a-c), together with sequence analysis, identified 8 antibodies that are described in the table below.

Table 3 summarises the results of the in vitro characterisation for the eight lead candidates.

TABLE 3
Summary of the in vitro Characterisation Assay Results for Eight Heparanase mAbs.

| | Binding to | | Inhibition of | | | | | | | |
|---|---|---|---|---|---|---|---|---|---|---|
| | direct | Binding to | recombinant human | | | | | | | |
| | coated | captured | heparanase % | | Binding to heparanase | | | mAb | Sequence information | |
| Antibody | heparanase^{a} | heparanase^{a} | Assay 1 | Assay 2 | Human^{b} | Cyno^{c} | Mouse^{d} | isotype | Light | Heavy |
| 7B9.1 | 3.78 | 6.0 | 63 | 54 | 0.68 | 1.06 | 0.58 | G4λ | V1-13/JL2 | VH3-33/D6-6/JH6B |
| 10E9.1 | 0.2 | 0.87 | 88 | 99 | 0.63 | 1.4 | 0.49 | G2κ | A20/JK3 | VH1-24/D4-17/JH6B |
| 14H12.3 | 3.69 | 3.27 | 58 | 85 | 0.61 | 0.95 | 0.14 | G2λ | V2-7/JL3 | VH3-33/D6-13/JH6B |
| 15A1.2 | 0.08 | 0.92 | 81 | 91 | 0.19 | 0.11 | 0.14 | G2κ | L5/JK1 | VH1-24/D3-3/JH6B |
| 15A11.1 | 0.07 | 0.5 | 75 | 87 | 0.21 | 0.14 | 0.17 | G2κ | A20/JK3 | VH1-24/D4-17/JH6B |
| 16E1.1 | 0.15 | 6.0 | 40 | 51 | 1.08 | 1.66 | 0.63 | G4λ | V2-1/JL2 | VH4-31/D6-13/JH3B |
| 16G1.1 | 3.63 | 3.82 | 44 | 53 | 1.84 | 1.64 | 0.61 | G4κ | A3/JK5 | VH3-33/NA/JH6B |
| 16G1.2 | | | | | | | | G4κ | L2/JK4 | VH3-33/NA/JH6B |

^{a}expressed as OD units
^{b}min 0.06, max 1.5 OD units
^{c}min 0.06, max 1.5 OD units
^{d}min 0.06, max 0.9 OD units

Based on the characteristics summarised in Table 3 and cross species heparanase inhibitory activity three antibodies, 10E9.1, 15A1.2 and 15A11.1, were selected for additional potency and affinity studies (see FIG. 4 and Example 13).

### Example 6

### Determination of Affinity Using Medium Resolution Biacore

### Medium Resolution Biacore™

For medium resolution Biacore™ tests, cloned and purified antibodies were used. Each of three purified antibodies (10E9.1, 15A1.2, and 15A11.1) were captured onto a high-density goat anti-human antibody surface over a CM4 Biacore™ chip. Recombinant Hpa1 was diluted to the desired concentration (1 nM and 10 nM) in 10 mM HEPES, 150 mM NaCl, 0.005% tween-20, 0.1 mg per ml BSA pH7.4 (25° C.) immediately prior to injection. Data were fit globally to a 1:1 interaction model with a term for mass transport included. The resulting K_{D} values are summarised below in Table 4.

TABLE 4
Affinity determination for purified antibodies by medium resolution
Biacore ™

| Antibody | kₐ (M⁻¹s⁻¹) | k_{d} (s⁻¹) | K_{D} (pM) |
|---|---|---|---|
| 10E9.1 | 2.06 × 10⁶ | 1.12 × 10⁻⁴ | 54.5 |
| 15A1.2 | 1.88 × 10⁶ | 1.05 × 10⁻⁴ | 85.9 |
| 15A11.1 | 3.60 × 10⁶ | 3.09 × 10⁻⁴ | 56.0 |

### Example 7

### Structural Analysis of Heparanase Antibodies

The variable heavy chains and the variable light chains of the antibodies were sequenced to determine their DNA sequences. The complete sequence information for the antibodies of the invention is provided in the sequence listing with nucleotide and amino acid sequences for each gamma and kappa chain combination. The variable heavy sequences were analyzed to determine the VH family, the D-region sequence and the J-region sequence. The sequences were then translated to determine the primary amino acid sequence and compared to the germline VH, D and J-region sequences to assess somatic hypermutations.

Table 5 is a table comparing the antibody heavy chain regions to their cognate germ line heavy chain region. Table 6 is a table comparing the antibody kappa light chain regions to their cognate germ line light chain region.

It should be appreciated that amino acid sequences among the sister clones collected from each hybridoma are identical. As an example, the heavy chain and light chain sequences for 7B9.1 would be identical to the sequences for 7B9.2. The variable (V) regions of immunoglobulin chains are encoded by multiple germ line DNA segments, which are joined into functional variable regions (V_{H}DJ_{H} or V_{K}J_{K}) during B-cell ontogeny. The molecular and genetic diversity of the antibody response to heparanase was studied in detail. These assays revealed several points specific to antibodies that specifically bind to heparanase.

According the sequencing data, the primary structure of the heavy and light chains of 10E9.1 and 15A11.1 are similar, but not identical. 15A1.2 is structurally different from 10E9.1 and 15A11.1.

Table 13 is a table showing the SEQ ID NO. of the heavy and light chain CDRs of 15A1.2, 10E9.1 and 15A11.1.

The skilled person will be aware that there are alternative methods of defining CDR boundaries. All CDR boundaries in Table 5, Table 6 and Table 13 are defined according to the Kabat definition.

TABLE 5
Heavy Chain Analysis

| | | | | | | | |
|---|---|---|---|---|---|---|---|
| Chain | SEQ ID | | | | | | |
| name | NO. | V | D | J | FR1 | CDR1 | FR2 |

| | | | | | |
|---|---|---|---|---|---|
| 10E9.1 | 33 | Germline | QVQLVQSGAEVKKPGASVKVSCKVSGYTLT | ELSMH | WVRQAPGKGLEWMG |

| | | | | | | |
|---|---|---|---|---|---|---|
| 2 | 1-24 | 4-17 | JH6 | QVQLVQSGAEVKKPGASVKVSCKVSGYSLT | ELSMH | WVRQAPGKGLEWMG |

| | | | | | |
|---|---|---|---|---|---|
| 15A11.1 | 34 | Germline | QVQLVQSGAEVKKPGASVKVSCKVSGYTLT | ELSMH | WVRQAPGKGLEWMG |

| | | | | | | |
|---|---|---|---|---|---|---|
| 10 | 1-24 | 4-17 | JH6 | QVQLVQSGAEVKKPGASVKVSCKVSGDTLT | ELSMH | WVRQTPGKGLEWMG |

| | | | | | |
|---|---|---|---|---|---|
| 16G1.1 | 35 | Germline | QVQLVESGGGVVQPGRSLRLSCAASGFTFS | SYGMH | WVRQAPGKGLEWVA |

| | | | | | | |
|---|---|---|---|---|---|---|
| 14 | 3-33 | 4-17 | JH6 | QVQLVESGGGVVQPGRSLRLSCAASGFTFS | SYGMH | WVRQAPGKGLEWVA |

| | | | | | |
|---|---|---|---|---|---|
| 16G1.2 | 35 | Germline | QVQLVESGGGVVQPGRSLRLSCAASGFTFS | SYGMH | WVRQAPGKGLEWVA |

| | | | | | | |
|---|---|---|---|---|---|---|
| 18 | 3-33 | 4-17 | JH6 | QVQLVESGGGVVQPGRSLRLSCAASGFTFS | SYGMH | WVRQAPGKGLEWVA |

| | | | | | |
|---|---|---|---|---|---|
| 15A1.2 | 36 | Germline | QVQLVQSGAEVKKPGASVKVSCKVSGYTLT | ELSMH | WVRQAPGKGLEWMG |

| | | | | | | |
|---|---|---|---|---|---|---|
| 6 | 1-24 | 3-3 | JH6 | QVQLVQSGAEVKKPGASVKVSCKVSGYTLT | ELSMH | WVRQAPGKGLEWMG |

| | | | | | |
|---|---|---|---|---|---|
| 7B9.1 | 37 | Germline | QVQLVESGGGVVQPGRSLRLSCAASGFTFS | SYGMH | WVRQAPGKGLEWVA |

| | | | | | | |
|---|---|---|---|---|---|---|
| 22 | 3-33 | 6-6 | JH3 | QVQLVESGGGVVQPGRSLRLSCAASGFTFS | SYGMH | WVRQAPGKGLEWVA |

| | | | | | |
|---|---|---|---|---|---|
| 16E1.1 | 38 | Germline | QVQLQESGPGLVKPSQTLSLTCTVSGGSIS | SGGYYWS | WIRQHPGKGLEWIG |

| | | | | | | |
|---|---|---|---|---|---|---|
| 30 | 4-31 | 6-13 | JH3 | QVQLQESGPGLVKPSQTLSLTCTVSGGSIS | SGGYYWS | WIRQHPGKGLEWIG |

| | | | | | |
|---|---|---|---|---|---|
| 14H12.3 | 39 | Germline | QVQLVESGGGVVQPGRSLRLSCAASGFTFS | SYGMH | WVRQAPGKGLEWVA |

| | | | | | | |
|---|---|---|---|---|---|---|
| 26 | 3-33 | 6-13 | JH6 | QVQLVESGGGVVQPGRSLRLSCAASGFTFS | SYGMH | WVRQAPGKGLEWVA |

| Chain | SEQ ID | | | | |
|---|---|---|---|---|---|
| name | NO. | CDR2 | FR3 | CDR3 | FR4 |
| 10E9.1 | 33 | GEDPEDGETIYAQKFQG | RVTMTEDTSTDTAYMELSSLASEDTAVYYCAT | ---DYGDY-YYYYYGMDV | WGQGTTVTVSS |
| | 2 | GFDPEDGEIIFAQKFQG | RVTLTEDISLDTAYMEVSSLASEDTAVYYCAT | EGHDSGDYVGYYYYGLDV | WGQGTTVTVSS |
| 15A11.1 | 34 | GFDPEDGETIYAQKFQG | RVTMTEDTSTDTAYMELSSLRSEDTAVYYCAT | --DYGD---YYYYYGMDV | WGQGTTVTVSS |
| | 10 | GFDPEDGETIYAQKFQG | RVTMTGDTSTDTAYMELSSLRSEDTAVYYCAT | EGDYGDSEEYYYYYGMDV | WGQGTTVTVSS |
| 16G1.1 | 35 | VIWYDGSNKYYADSVKG | RFTISRDNSKNTLYLQMNSLRAEDTAVYYCAR | DYGDYYYYYYGMDV | WGQGTTVTVSS |
| | 14 | VIWYDGSNKYYADSVKG | RFTISRDNSKNTLNLQMNSLRAEDTAVYYCGR | DPGTYHYYYHGMDV | WGQGTTVTVSS |
| 16G1.2 | 35 | VIWYDGSNKYYADSVKG | RFTISRDNSKNTLYLQMNSLRAEDTAVYYCAR | DYGDYYYYYYGMDV | WGQGTTVTVSS |
| | 18 | VIWYDGSNKYYADSVKG | RFTISRDNSKNTLNLQMNSLRAEDTAVYYCGR | DPGTYHYYYHGMDV | WGQGTTVTVSS |
| 15A1.2 | 36 | GFDPEDGETIYAQKFQG | RVTMTEDTSTDTAYMELSSLASEDTAVYYCAT | -LRFLEWLL-GMDV | WGQGTTVTVSS |
| | 6 | GFDPEDGETIFAQKFQG | RVTMTEDTSTDTAYMELSSLRSEDTAVYYCAT | GLRFLEWFLWGWDV | WGQGTTVTVSS |
| 7B9.1 | 37 | VIWYDGSNKYYADSVKG | RFTISRDNSKNTLYLQMNSLRAEDTAVYYCAR | ----YSSS-AFDI | WGQGTMVTVSS |
| | 22 | VIWYDGSNKYYADSVKG | RFTISRDNSKNTLYLQMNSLRAEDTAVYYCAR | EGAPYSSSDAFDI | WGQGTMVTVSS |
| 16E1.1 | 38 | YIYYSGSTYYNPSLKS | RVTISVDTSKNQFSLKLSSVTAADTAVYYCAR | -YSS----AFDI | WGQGTMVTVSS |
| | 30 | FIYYSGSTFYNPSLKS | RITISVDTSKNQFSLKLSSVTAADTAVYYCAR | PYSSSWYGAFDI | WGQGTMVTVSS |
| 14H12.3 | 39 | VIWYDGSNKYYADSVKG | RFTISRDNSKNTLYLQMNSLRAEDTAVYYCAR | --SSSW--YYGMDV | WGQGTTVTVSS |
| | 26 | VIWYDGSNKDYADSVKG | RFTISRDNSKNTLYLQMNSLRAEDTAVYYCAR | EGSTSWTDYYGMDV | WGQGTTVTVSS |

TABLE 6
Light Chain Analysis

| | | | | | | |
|---|---|---|---|---|---|---|
| Chain | SEQ ID | | | | | |
| name | NO. | V | J | FR1 | CDR1 | FR2 |

| | | | | | |
|---|---|---|---|---|---|
| 10E9.1 | 40 | Germline | DIQMTQSPSSLSASVGDRVTITC | RASQGISNYLA | WYQQKPGKVPKLLIY |

| | | | | | |
|---|---|---|---|---|---|
| 4 | A20 | JK3 | DIQMTQSPSSLSASVGDRVTITC | RASQDIRNFLA | WYQQKPGKLPKLLIY |

| | | | | | |
|---|---|---|---|---|---|
| 15A11.1 | 40 | Germline | DIQMTQSPSSLSASVGDRVTITC | RASQGISNYLA | WYQQKPGKVPKLLIY |

| | | | | | |
|---|---|---|---|---|---|
| 12 | A20 | JK3 | DIQMTQSPSSLSASVGDRVTITC | RASQGISNYLA | WYQQKPGKVPKLLIY |

| | | | | | |
|---|---|---|---|---|---|
| 16G1.1 | 41 | Germline | DIVMTQSPLSLPVTPGEPASISC | RSSQSLLHSNGYNYLD | WYLQKPGQSPQLLIY |

| | | | | | |
|---|---|---|---|---|---|
| 16 | A3/A19 | JK5 | YIVMTQSPLSLPVTPGEPASISC | RSSQSLLHSNGYNFLD | WYLQKPGQSPQLLIY |

| | | | | | |
|---|---|---|---|---|---|
| 16G1.2 | 42 | Germline | EIVMTQSPATLSVSPGERATLSC | RASQSVSSNLA | WYQQKPGQAPRLLIY |

| | | | | | |
|---|---|---|---|---|---|
| 20 | L2 | JK4 | QIVMTQSPATLSVSPGERATLSC | RASQSVSRNLA | WYQQKPGQSPRLLIY |

| | | | | | |
|---|---|---|---|---|---|
| 15A1.2 | 43 | Germline | DIQMTQSPSSVSASVGDRVTITC | RASQGISSWLA | WYQQKPGKAPKLLIY |

| | | | | | |
|---|---|---|---|---|---|
| 8 | L5 | JK1 | DIQMTQSPSSVSASVGDRVTITC | RASQGISNWLA | WYQQTPGKAPQLLIY |

| | | | | | |
|---|---|---|---|---|---|
| 7B9.1 | 44 | Germline | QSVLTQPPSVSGAPGQRVTISC | TGSSSNIGAGYDVH | WYQQLPGTAPKLLIY |

| | | | | | |
|---|---|---|---|---|---|
| 24 | 1e | JL2/JL3 | QSVLTQPPSVSGAPGQRVTISC | TGSSSNIGANYDVH | WYQQLPGTAPKLLIY |

| | | | | | |
|---|---|---|---|---|---|
| 16E1.1 | 45 | Germline | SYELTQPPSVSVSPGQTASITC | SGDKLGDKYAC | WYQQKPGQSPVLVIY |

| | | | | | |
|---|---|---|---|---|---|
| 32 | 3r | JL2/JL3 | SYELTQPPSVSVSPGRTASITC | SEDKLGNKYAY | WYQQKPGQSPVLVIY |

| | | | | | |
|---|---|---|---|---|---|
| 14H12.3 | 46 | Germline | SYELTQPPSVSVSPGQTARITC | SGDALPKKYAY | WYQQKSGQAPVLVIY |

| | | | | | |
|---|---|---|---|---|---|
| 28 | 3p | JL2/JL3 | SYELTQPPSVSVSPGQTARITC | SGDALPKRYAY | WYQQKSGQALVLVIY |

| Chain | SEQ ID | | | | |
|---|---|---|---|---|---|
| name | NO. | CDR2 | FR3 | CDR3 | FR4 |
| 10E9.1 | 40 | AASTLQS | GVPSRFSGSGSGTDFTLTISSLQPEDVATYYC | QKYNSAPFT | FGPGTKVDIK |
| | 4 | AASTLQS | GVPSRFSGSGSGTDFTLTISSLQPEDVASYYC | QSHNSVPFT | FGPGTKVDIR |
| 15A11.1 | 40 | AASTLQS | GVPSRFSGSGSGTDFTLTISSLQPEDVATYYC | QKYNSAPFT | FGPGTKVDIK |
| | 12 | AASTLQS | GVPSRFSGSGSGTDFTLTISSLQPEDVATYYC | QKYNRAPFT | FGPGTKVDIK |
| 16G1.1 | 41 | LGSNRAS | GVPDRFSGSGSGTDFTLKISRVEAEDVGVYYC | MQALQTPIT | FGQGTRLEIK |
| | 16 | LGSNRAS | GVPDRFSGSGSGTDFTLKINRVEAEDVGVYYC | MQALQTPIT | FGQGTRLEIK |
| 16G1.2 | 42 | GASTRAT | GIPARFSGSGSGTEFTLTISSLQSEDFAVYYC | QQYNNWPLT | FGGGTKVEIK |
| | 20 | GASTRAT | GFPARISGSGSGTEFTLTISSLQSEDFAVYYC | QQYNHWPQT | FGGGTEVEIK |
| 15A1.2 | 43 | AASSLQS | GVPSRFSGSGSGTDFTLTISSLQPEDFATYYC | QQANSFPWT | FGQGTKVEIK |
| | 8 | GASSLQS | GVPSRFSGSGSGTDFTLTISSLQPEDFATYYC | QQADSFPPT | FGQGTKVEIK |
| 7B9.1 | 44 | GNSNRPS | GVPDRFSGSKSGTSASLAITGLQAEDEADYYC | QSYDSSLSGVV | FGGGTKLTVL |
| | 24 | GDNNRPS | GVPDRFSGSKSGTSASLAITGLQAEDEADYYC | QSYDSSLSGSV | FGGGTKLTVL |
| 16E1.1 | 45 | QDSKRPS | GIPERFSGSNSGNTATLTISGTQAMDEADYYC | QAWDSSTAVV | FGGGTKLTVL |
| | 32 | QDSERPS | GIPERFSGSKSGNTATLTISGTQANDEADYYC | QAWDSST-VV | FGGGTKLTVL |
| 14H12.3 | 46 | EDSKRPS | GIPERFSGSSSGTMATLTISGAQVEDEADYYC | YSTDSSGNHVV | FGGGTKLTVL |
| | 28 | EDSKRPS | GIPERFSGSSSGTMATLTISGAQVEDEADYYC | YSIDSSGNHGV | FGGGTKLTVL |

TABLE 13
SEQ ID NO. s of the heavy and light chain CDRs of
15A1.2, 10E9 and 15A11.1.

| CHAIN | | SEQ ID NO. | | |
|---|---|---|---|---|
| NAME | CHAIN | CDR1 | CDR2 | CDR3 |
| 10E9 | Heavy | 47 | 48 | 49 |
| | Light | 56 | 57 | 58 |
| 15A11.1 | Heavy | 50 | 51 | 52 |
| | Light | 59 | 60 | 61 |
| 15A1.2 | Heavy | 53 | 54 | 55 |
| | Light | 62 | 63 | 64 |

It should also be appreciated that where a particular antibody differs from its respective germline sequence at the amino acid level, the antibody sequence can be mutated back to the germline sequence. Such corrective mutations can occur at one, two, three or more positions, or a combination of any of the mutated positions, using standard molecular biological techniques. By way of non-limiting example, Table 10 shows that the light chain sequence of mAb 15A1.2 (SEQ ID NO.: 8) differs from the corresponding germline sequence (SEQ ID NO.:43) through a Ser to Asn mutation (difference 1) in the CDR1 region, a Lys to Thr mutation (difference 2) in the FR2 region, a Lys to Gln mutation (difference 3) in the FR2 region, an Ala to Gly mutation (difference 4) in the CDR2 region, an Asn to Asp mutation (difference 5) in the CDR3 region and a Try to Pro mutation (difference 6) in the CDR3 region. Thus, the amino acid or nucleotide sequence encoding the light chain of mAb 15A1.2 can be modified to yield the germline sequence at the site of difference 1. Further, the amino acid or nucleotide sequence encoding the light chain of mAb 15A1.2 can be modified to yield the germline sequence at the site of difference 1, difference 2, difference 3, difference 4, difference 5 or difference 6. Still further, the amino acid or nucleotide sequence encoding the light chain of mAb 15A1.2 can be modified to yield the germline sequence at the site of both difference 1 and difference 2, or any other combination of the two, three, four, five or six differences to yield the germline sequence at those particular sites.

Tables 7-12 below illustrate the positions of such variations from the germline for monoclonal antibodies 15A1.2, 15A11.1 or 10E9.1. Each row represents a combination of residues at the position indicated.

TABLE 7a
Exemplary Mutations of mAB 10E9.1 Heavy chain
(SEQ ID NO: 2) to Germline at the Indicated Residue Number

| 28 | 70 | 74 | 76 | 83 |
|---|---|---|---|---|
| S | L | I | L | V |
| T | L | I | L | V |
| S | M | I | L | V |
| T | M | I | L | V |
| S | L | T | L | V |
| T | L | T | L | V |
| S | M | T | L | V |
| T | M | T | L | V |
| S | L | I | T | V |
| T | L | I | T | V |
| S | M | I | T | V |
| T | M | I | T | V |
| S | L | T | T | V |
| T | L | T | T | V |
| S | M | T | T | V |
| T | M | T | T | V |
| S | L | I | L | L |
| T | L | I | L | L |
| S | M | I | L | L |
| T | M | I | L | L |
| S | L | T | L | L |
| T | L | T | L | L |
| S | M | T | L | L |
| T | M | T | L | L |
| S | L | I | T | L |
| T | L | I | T | L |
| S | M | I | T | L |
| T | M | I | T | L |
| S | L | T | T | L |
| T | L | T | T | L |
| S | M | T | T | L |
| T | M | T | T | L |

TABLE 7b
Exemplary Mutations of mAB 10E9.1 Heavy Chain (SEQ ID NO: 2) to
Germline at the Indicated Residue Number

| 34 | 58 | 60 | 99 | 100 | 101 | 103 | 107 | 108 | 114 |
|---|---|---|---|---|---|---|---|---|---|
| I | I | F | E | G | H | S | V | G | L |
| M | I | F | E | G | H | S | V | G | L |
| I | T | F | E | G | H | S | V | G | L |
| M | T | F | E | G | H | S | V | G | L |
| I | I | Y | E | G | H | S | V | G | L |
| M | I | Y | E | G | H | S | V | G | L |
| I | T | Y | E | G | H | S | V | G | L |
| M | T | Y | E | G | H | S | V | G | L |
| I | I | F | — | G | H | S | V | G | L |
| M | I | F | — | G | H | S | V | G | L |
| I | T | F | — | G | H | S | V | G | L |
| M | T | F | — | G | H | S | V | G | L |
| I | I | Y | — | G | H | S | V | G | L |
| M | I | Y | — | G | H | S | V | G | L |
| I | T | Y | — | G | H | S | V | G | L |
| M | T | Y | — | G | H | S | V | G | L |
| I | I | F | E | — | H | S | V | G | L |
| M | I | F | E | — | H | S | V | G | L |
| I | T | F | E | — | H | S | V | G | L |
| M | T | F | E | — | H | S | V | G | L |
| I | I | Y | E | — | H | S | V | G | L |
| M | I | Y | E | — | H | S | V | G | L |
| I | T | Y | E | — | H | S | V | G | L |
| M | T | Y | E | — | H | S | V | G | L |
| I | I | F | — | — | H | S | V | G | L |
| M | I | F | — | — | H | S | V | G | L |
| I | T | F | — | — | H | S | V | G | L |
| M | T | F | — | — | H | S | V | G | L |
| I | I | Y | — | — | H | S | V | G | L |
| M | I | Y | — | — | H | S | V | G | L |
| I | T | Y | — | — | H | S | V | G | L |
| M | T | Y | — | — | H | S | V | G | L |
| I | I | F | E | G | — | S | V | G | L |
| M | I | F | E | G | — | S | V | G | L |
| I | T | F | E | G | — | S | V | G | L |
| M | T | F | E | G | — | S | V | G | L |
| I | I | Y | E | G | — | S | V | G | L |
| M | I | Y | E | G | — | S | V | G | L |
| I | T | Y | E | G | — | S | V | G | L |
| M | T | Y | E | G | — | S | V | G | L |
| I | I | F | — | G | — | S | V | G | L |
| M | I | F | — | G | — | S | V | G | L |
| I | T | F | — | G | — | S | V | G | L |
| M | T | F | — | G | — | S | V | G | L |
| I | I | Y | — | G | — | S | V | G | L |
| M | I | Y | — | G | — | S | V | G | L |
| I | T | Y | — | G | — | S | V | G | L |
| M | T | Y | — | G | — | S | V | G | L |
| I | I | F | E | — | — | S | V | G | L |
| M | I | F | E | — | — | S | V | G | L |
| I | T | F | E | — | — | S | V | G | L |
| M | T | F | E | — | — | S | V | G | L |
| I | I | Y | E | — | — | S | V | G | L |
| M | I | Y | E | — | — | S | V | G | L |
| I | T | Y | E | — | — | S | V | G | L |
| M | T | Y | E | — | — | S | V | G | L |
| I | I | F | — | — | — | S | V | G | L |
| M | I | F | — | — | — | S | V | G | L |
| I | T | F | — | — | — | S | V | G | L |
| M | T | F | — | — | — | S | V | G | L |
| I | I | Y | — | — | — | S | V | G | L |
| M | I | Y | — | — | — | S | V | G | L |
| I | T | Y | — | — | — | S | V | G | L |
| M | T | Y | — | — | — | S | V | G | L |
| I | I | F | E | G | H | Y | V | G | L |
| M | I | F | E | G | H | Y | V | G | L |
| I | T | F | E | G | H | Y | V | G | L |
| M | T | F | F | G | H | Y | V | G | L |
| I | I | Y | E | G | H | Y | V | G | L |
| M | I | Y | E | G | H | Y | V | G | L |
| I | T | Y | E | G | H | Y | V | G | L |
| M | T | Y | E | G | H | Y | V | G | L |
| I | I | F | — | G | H | Y | V | G | L |
| M | I | F | — | G | H | Y | V | G | L |
| I | T | F | — | G | H | Y | V | G | L |
| M | T | F | — | G | H | Y | V | G | L |
| I | I | Y | — | G | H | Y | V | G | L |
| M | I | Y | — | G | H | Y | V | G | L |
| I | T | Y | — | G | H | Y | V | G | L |
| M | T | Y | — | G | H | Y | V | G | L |
| I | I | F | E | — | H | Y | V | G | L |
| M | I | F | E | — | H | Y | V | G | L |
| I | T | F | E | — | H | Y | V | G | L |
| M | T | F | E | — | H | Y | V | G | L |
| I | I | Y | E | — | H | Y | V | G | L |
| M | I | Y | E | — | H | Y | V | G | L |
| I | T | Y | E | — | H | Y | V | G | L |
| M | T | Y | E | — | H | Y | V | G | L |
| I | I | F | — | — | H | Y | V | G | L |
| M | I | F | — | — | H | Y | V | G | L |
| I | T | F | — | — | H | Y | V | G | L |
| M | T | F | — | — | H | Y | V | G | L |
| I | I | Y | — | — | H | Y | V | G | L |
| M | I | Y | — | — | H | Y | V | G | L |
| I | T | Y | — | — | H | Y | V | G | L |
| M | T | Y | — | — | H | Y | V | G | L |
| I | I | F | E | G | — | Y | V | G | L |
| M | I | F | E | G | — | Y | V | G | L |
| I | T | F | E | G | — | Y | V | G | L |
| M | T | F | E | G | — | Y | V | G | L |
| I | I | Y | E | G | — | Y | V | G | L |
| M | I | Y | E | G | — | Y | V | G | L |
| I | T | Y | E | G | — | Y | V | G | L |
| M | T | Y | E | G | — | Y | V | G | L |
| I | I | F | — | G | — | Y | V | G | L |
| M | I | F | — | G | — | Y | V | G | L |
| I | T | F | — | G | — | Y | V | G | L |
| M | T | F | — | G | — | Y | V | G | L |
| I | I | Y | — | G | — | Y | V | G | L |
| M | I | Y | — | G | — | Y | V | G | L |
| I | T | Y | — | G | — | Y | V | G | L |
| M | T | Y | — | G | — | Y | V | G | L |
| I | I | F | E | — | — | Y | V | G | L |
| M | I | F | E | — | — | Y | V | G | L |
| I | T | F | E | — | — | Y | V | G | L |
| M | T | F | E | — | — | Y | V | G | L |
| I | I | Y | E | — | — | Y | V | G | L |
| M | I | Y | E | — | — | Y | V | G | L |
| I | T | Y | E | — | — | Y | V | G | L |
| M | T | Y | E | — | — | Y | V | G | L |
| I | I | F | — | — | — | Y | V | G | L |
| M | I | F | — | — | — | Y | V | G | L |
| I | T | F | — | — | — | Y | V | G | L |
| M | T | F | — | — | — | Y | V | G | L |
| I | I | Y | — | — | — | Y | V | G | L |
| M | I | Y | — | — | — | Y | V | G | L |
| I | T | Y | — | — | — | Y | V | G | L |
| M | T | Y | — | — | — | Y | V | G | L |
| I | I | F | E | G | H | S | — | G | L |
| M | I | F | E | G | H | S | — | G | L |
| I | T | F | E | G | H | S | — | G | L |
| M | T | F | E | G | H | S | — | G | L |
| I | I | Y | E | G | H | S | — | G | L |
| M | I | Y | E | G | H | S | — | G | L |
| I | T | Y | E | G | H | S | — | G | L |
| M | T | Y | E | G | H | S | — | G | L |
| I | I | F | — | G | H | S | — | G | L |
| M | I | F | — | G | H | S | — | G | L |
| I | T | F | — | G | H | S | — | G | L |
| M | T | F | — | G | H | S | — | G | L |
| I | I | Y | — | G | H | S | — | G | L |
| M | I | Y | — | G | H | S | — | G | L |
| I | T | Y | — | G | H | S | — | G | L |
| M | T | Y | — | G | H | S | — | G | L |
| I | I | F | E | — | H | S | — | G | L |
| M | I | F | E | — | H | S | — | G | L |
| I | T | F | E | — | H | S | — | G | L |
| M | T | F | E | — | H | S | — | G | L |
| I | I | Y | E | — | H | S | — | G | L |
| M | I | Y | E | — | H | S | — | G | L |
| I | T | Y | E | — | H | S | — | G | L |
| M | T | Y | E | — | H | S | — | G | L |
| I | I | F | — | — | H | S | — | G | L |
| M | I | F | — | — | H | S | — | G | L |
| I | T | F | — | — | H | S | — | G | L |
| M | T | F | — | — | H | S | — | G | L |
| I | I | Y | — | — | H | S | — | G | L |
| M | I | Y | — | — | H | S | — | G | L |
| I | T | Y | — | — | H | S | — | G | L |
| M | T | Y | — | — | H | S | — | G | L |
| I | I | F | E | G | — | S | — | G | L |
| M | I | F | E | G | — | S | — | G | L |
| I | T | F | E | G | — | S | — | G | L |
| M | T | F | E | G | — | S | — | G | L |
| I | I | Y | E | G | — | S | — | G | L |
| M | I | Y | E | G | — | S | — | G | L |
| I | T | Y | E | G | — | S | — | G | L |
| M | T | Y | E | G | — | S | — | G | L |
| I | I | F | — | G | — | S | — | G | L |
| M | I | F | — | G | — | S | — | G | L |
| I | T | F | — | G | — | S | — | G | L |
| M | T | F | — | G | — | S | — | G | L |
| I | I | Y | — | G | — | S | — | G | L |
| M | I | Y | — | G | — | S | — | G | L |
| I | T | Y | — | G | — | S | — | G | L |
| M | T | Y | — | G | — | S | — | G | L |
| I | I | F | E | — | — | S | — | G | L |
| M | I | F | E | — | — | S | — | G | L |
| I | T | F | E | — | — | S | — | G | L |
| M | T | F | E | — | — | S | — | G | L |
| I | I | Y | E | — | — | S | — | G | L |
| M | I | Y | E | — | — | S | — | G | L |
| I | T | Y | E | — | — | S | — | G | L |
| M | T | Y | E | — | — | S | — | G | L |
| I | I | F | — | — | — | S | — | G | L |
| M | I | F | — | — | — | S | — | G | L |
| I | T | F | — | — | — | S | — | G | L |
| M | T | F | — | — | — | S | — | G | L |
| I | I | Y | — | — | — | S | — | G | L |
| M | I | Y | — | — | — | S | — | G | L |
| I | T | Y | — | — | — | S | — | G | L |
| M | T | Y | — | — | — | S | — | G | L |
| I | I | F | E | G | H | Y | — | G | L |
| M | I | F | E | G | H | Y | — | G | L |
| I | T | F | E | G | H | Y | — | G | L |
| M | T | F | E | G | H | Y | — | G | L |
| I | I | Y | E | G | H | Y | — | G | L |
| M | I | Y | E | G | H | Y | — | G | L |
| I | T | Y | E | G | H | Y | — | G | L |
| M | T | Y | E | G | H | Y | — | G | L |
| I | I | F | — | G | H | Y | — | G | L |
| M | I | F | — | G | H | Y | — | G | L |
| I | T | F | — | G | H | Y | — | G | L |
| M | T | F | — | G | H | Y | — | G | L |
| I | I | Y | — | G | H | Y | — | G | L |
| M | I | Y | — | G | H | Y | — | G | L |
| I | T | Y | — | G | H | Y | — | G | L |
| M | T | Y | — | G | H | Y | — | G | L |
| I | I | F | E | — | H | Y | — | G | L |
| M | I | F | E | — | H | Y | — | G | L |
| I | T | F | E | — | H | Y | — | G | L |
| M | T | F | E | — | H | Y | — | G | L |
| I | I | Y | E | — | H | Y | — | G | L |
| M | I | Y | E | — | H | Y | — | G | L |
| I | T | Y | E | — | H | Y | — | G | L |
| M | T | Y | E | — | H | Y | — | G | L |
| I | I | F | — | — | H | Y | — | G | L |
| M | I | F | — | — | H | Y | — | G | L |
| I | T | F | — | — | H | Y | — | G | L |
| M | T | F | — | — | H | Y | — | G | L |
| I | I | Y | — | — | H | Y | — | G | L |
| M | I | Y | — | — | H | Y | — | G | L |
| I | T | Y | — | — | H | Y | — | G | L |
| M | T | Y | — | — | H | Y | — | G | L |
| I | I | F | E | G | — | Y | — | G | L |
| M | I | F | E | G | — | Y | — | G | L |
| I | T | F | E | G | — | Y | — | G | L |
| M | T | F | E | G | — | Y | — | G | L |
| I | I | Y | E | G | — | Y | — | G | L |
| M | I | Y | E | G | — | Y | — | G | L |
| I | T | Y | E | G | — | Y | — | G | L |
| M | T | Y | E | G | — | Y | — | G | L |
| I | I | F | — | G | — | Y | — | G | L |
| M | I | F | — | G | — | Y | — | G | L |
| I | T | F | — | G | — | Y | — | G | L |
| M | T | F | — | G | — | Y | — | G | L |
| I | I | Y | — | G | — | Y | — | G | L |
| M | I | Y | — | G | — | Y | — | G | L |
| I | T | Y | — | G | — | Y | — | G | L |
| M | T | Y | — | G | — | Y | — | G | L |
| I | I | F | E | — | — | Y | — | G | L |
| M | I | F | E | — | — | Y | — | G | L |
| I | T | F | E | — | — | Y | — | G | L |
| M | T | F | E | — | — | Y | — | G | L |
| I | I | Y | E | — | — | Y | — | G | L |
| M | I | Y | E | — | — | Y | — | G | L |
| I | T | Y | E | — | — | Y | — | G | L |
| M | T | Y | E | — | — | Y | — | G | L |
| I | I | F | — | — | — | Y | — | G | L |
| M | I | F | — | — | — | Y | — | G | L |
| I | T | F | — | — | — | Y | — | G | L |
| M | T | F | — | — | — | Y | — | G | L |
| I | I | Y | — | — | — | Y | — | G | L |
| M | I | Y | — | — | — | Y | — | G | L |
| I | T | Y | — | — | — | Y | — | G | L |
| M | T | Y | — | — | — | Y | — | G | L |
| I | I | F | E | G | H | S | V | Y | L |
| M | I | F | E | G | H | S | V | Y | L |
| I | T | F | E | G | H | S | V | Y | L |
| M | T | F | E | G | H | S | V | Y | L |
| I | I | Y | E | G | H | S | V | Y | L |
| M | I | Y | E | G | H | S | V | Y | L |
| I | T | Y | E | G | H | S | V | Y | L |
| M | T | Y | E | G | H | S | V | Y | L |
| I | I | F | — | G | H | S | V | Y | L |
| M | I | F | — | G | H | S | V | Y | L |
| I | T | F | — | G | H | S | V | Y | L |
| M | T | F | — | G | H | S | V | Y | L |
| I | I | Y | — | G | H | S | V | Y | L |
| M | I | Y | — | G | H | S | V | Y | L |
| I | T | Y | — | G | H | S | V | Y | L |
| M | T | Y | — | G | H | S | V | Y | L |
| I | I | F | E | — | H | S | V | Y | L |
| M | I | F | E | — | H | S | V | Y | L |
| I | T | F | E | — | H | S | V | Y | L |
| M | T | F | E | — | H | S | V | Y | L |
| I | I | Y | E | — | H | S | V | Y | L |
| M | I | Y | E | — | H | S | V | Y | L |
| I | T | Y | E | — | H | S | V | Y | L |
| M | T | Y | E | — | H | S | V | Y | L |
| I | I | F | — | — | H | S | V | Y | L |
| M | I | F | — | — | H | S | V | Y | L |
| I | T | F | — | — | H | S | V | Y | L |
| M | T | F | — | — | H | S | V | Y | L |
| I | I | Y | — | — | H | S | V | Y | L |
| M | I | Y | — | — | H | S | V | Y | L |
| I | T | Y | — | — | H | S | V | Y | L |
| M | T | Y | — | — | H | S | V | Y | L |
| I | I | F | E | G | — | S | V | Y | L |
| M | I | F | E | G | — | S | V | Y | L |
| I | T | F | E | G | — | S | V | Y | L |
| M | T | F | E | G | — | S | V | Y | L |
| I | I | Y | E | G | — | S | V | Y | L |
| M | I | Y | E | G | — | S | V | Y | L |
| I | T | Y | E | G | — | S | V | Y | L |
| M | T | Y | E | G | — | S | V | Y | L |
| I | I | F | — | G | — | S | V | Y | L |
| M | I | F | — | G | — | S | V | Y | L |
| I | T | F | — | G | — | S | V | Y | L |
| M | T | F | — | G | — | S | V | Y | L |
| I | I | Y | — | G | — | S | V | Y | L |
| M | I | Y | — | G | — | S | V | Y | L |
| I | T | Y | — | G | — | S | V | Y | L |
| M | T | Y | — | G | — | S | V | Y | L |
| I | I | F | E | — | — | S | V | Y | L |
| M | I | F | E | — | — | S | V | Y | L |
| I | T | F | E | — | — | S | V | Y | L |
| M | T | F | E | — | — | S | V | Y | L |
| I | I | Y | E | — | — | S | V | Y | L |
| M | I | Y | E | — | — | S | V | Y | L |
| I | T | Y | E | — | — | S | V | Y | L |
| M | T | Y | E | — | — | S | V | Y | L |
| I | I | F | — | — | — | S | V | Y | L |
| M | I | F | — | — | — | S | V | Y | L |
| I | T | F | — | — | — | S | V | Y | L |
| M | T | F | — | — | — | S | V | Y | L |
| I | I | Y | — | — | — | S | V | Y | L |
| M | I | Y | — | — | — | S | V | Y | L |
| I | T | Y | — | — | — | S | V | Y | L |
| M | T | Y | — | — | — | S | V | Y | L |
| I | I | F | E | G | H | Y | V | Y | L |
| M | I | F | E | G | H | Y | V | Y | L |
| I | T | F | E | G | H | Y | V | Y | L |
| M | T | F | E | G | H | Y | V | Y | L |
| I | I | Y | E | G | H | Y | V | Y | L |
| M | I | Y | E | G | H | Y | V | Y | L |
| I | T | Y | E | G | H | Y | V | Y | L |
| M | T | Y | E | G | H | Y | V | Y | L |
| I | I | F | — | G | H | Y | V | Y | L |
| M | I | F | — | G | H | Y | V | Y | L |
| I | T | F | — | G | H | Y | V | Y | L |
| M | T | F | — | G | H | Y | V | Y | L |
| I | I | Y | — | G | H | Y | V | Y | L |
| M | I | Y | — | G | H | Y | V | Y | L |
| I | T | Y | — | G | H | Y | V | Y | L |
| M | T | Y | — | G | H | Y | V | Y | L |
| I | I | F | E | — | H | Y | V | Y | L |
| M | I | F | E | — | H | Y | V | Y | L |
| I | T | F | E | — | H | Y | V | Y | L |
| M | T | F | E | — | H | Y | V | Y | L |
| I | I | Y | E | — | H | Y | V | Y | L |
| M | I | Y | E | — | H | Y | V | Y | L |
| I | T | Y | E | — | H | Y | V | Y | L |
| M | T | Y | E | — | H | Y | V | Y | L |
| I | I | F | — | — | H | Y | V | Y | L |
| M | I | F | — | — | H | Y | V | Y | L |
| I | T | F | — | — | H | Y | V | Y | L |
| M | T | F | — | — | H | Y | V | Y | L |
| I | I | Y | — | — | H | Y | V | Y | L |
| M | I | Y | — | — | H | Y | V | Y | L |
| I | T | Y | — | — | H | Y | V | Y | L |
| M | T | Y | — | — | H | Y | V | Y | L |
| I | I | F | E | G | — | Y | V | Y | L |
| M | I | F | E | G | — | Y | V | Y | L |
| I | T | F | E | G | — | Y | V | Y | L |
| M | T | F | E | G | — | Y | V | Y | L |
| I | I | Y | E | G | — | Y | V | Y | L |
| M | I | Y | E | G | — | Y | V | Y | L |
| I | T | Y | E | G | — | Y | V | Y | L |
| M | T | Y | E | G | — | Y | V | Y | L |
| I | I | F | — | G | — | Y | V | Y | L |
| M | I | F | — | G | — | Y | V | Y | L |
| I | T | F | — | G | — | Y | V | Y | L |
| M | T | F | — | G | — | Y | V | Y | L |
| I | I | Y | — | G | — | Y | V | Y | L |
| M | I | Y | — | G | — | Y | V | Y | L |
| I | T | Y | — | G | — | Y | V | Y | L |
| M | T | Y | — | G | — | Y | V | Y | L |
| I | I | F | E | — | — | Y | V | Y | L |
| M | I | F | E | — | — | Y | V | Y | L |
| I | T | F | E | — | — | Y | V | Y | L |
| M | T | F | E | — | — | Y | V | Y | L |
| I | I | Y | E | — | — | Y | V | Y | L |
| M | I | Y | E | — | — | Y | V | Y | L |
| I | T | Y | E | — | — | Y | V | Y | L |
| M | T | Y | E | — | — | Y | V | Y | L |
| I | I | F | — | — | — | Y | V | Y | L |
| M | I | F | — | — | — | Y | V | Y | L |
| I | T | F | — | — | — | Y | V | Y | L |
| M | T | F | — | — | — | Y | V | Y | L |
| I | I | Y | — | — | — | Y | V | Y | L |
| M | I | Y | — | — | — | Y | V | Y | L |
| I | T | Y | — | — | — | Y | V | Y | L |
| M | T | Y | — | — | — | Y | V | Y | L |
| I | I | F | E | G | H | S | — | Y | L |
| M | I | F | E | G | H | S | — | Y | L |
| I | T | F | E | G | H | S | — | Y | L |
| M | T | F | E | G | H | S | — | Y | L |
| I | I | Y | E | G | H | S | — | Y | L |
| M | I | Y | E | G | H | S | — | Y | L |
| I | T | Y | E | G | H | S | — | Y | L |
| M | T | Y | E | G | H | S | — | Y | L |
| I | I | F | — | G | H | S | — | Y | L |
| M | I | F | — | G | H | S | — | Y | L |
| I | T | F | — | G | H | S | — | Y | L |
| M | T | F | — | G | H | S | — | Y | L |
| I | I | Y | — | G | H | S | — | Y | L |
| M | I | Y | — | G | H | S | — | Y | L |
| I | T | Y | — | G | H | S | — | Y | L |
| M | T | Y | — | G | H | S | — | Y | L |
| I | I | F | E | — | H | S | — | Y | L |
| M | I | F | E | — | H | S | — | Y | L |
| I | T | F | E | — | H | S | — | Y | L |
| M | T | F | E | — | H | S | — | Y | L |
| I | I | Y | E | — | H | S | — | Y | L |
| M | I | Y | E | — | H | S | — | Y | L |
| I | T | Y | E | — | H | S | — | Y | L |
| M | T | Y | E | — | H | S | — | Y | L |
| I | I | F | — | — | H | S | — | Y | L |
| M | I | F | — | — | H | S | — | Y | L |
| I | T | F | — | — | H | S | — | Y | L |
| M | T | F | — | — | H | S | — | Y | L |
| I | I | Y | — | — | H | S | — | Y | L |
| M | I | Y | — | — | H | S | — | Y | L |
| I | T | Y | — | — | H | S | — | Y | L |
| M | T | Y | — | — | H | S | — | Y | L |
| I | I | F | E | G | — | S | — | Y | L |
| M | I | F | E | G | — | S | — | Y | L |
| I | T | F | E | G | — | S | — | Y | L |
| M | T | F | E | G | — | S | — | Y | L |
| I | I | Y | E | G | — | S | — | Y | L |
| M | I | Y | E | G | — | S | — | Y | L |
| I | T | Y | E | G | — | S | — | Y | L |
| M | T | Y | E | G | — | S | — | Y | L |
| I | I | F | — | G | — | S | — | Y | L |
| M | I | F | — | G | — | S | — | Y | L |
| I | T | F | — | G | — | S | — | Y | L |
| M | T | F | — | G | — | S | — | Y | L |
| I | I | Y | — | G | — | S | — | Y | L |
| M | I | Y | — | G | — | S | — | Y | L |
| I | T | Y | — | G | — | S | — | Y | L |
| M | T | Y | — | G | — | S | — | Y | L |
| I | I | F | E | — | — | S | — | Y | L |
| M | I | F | E | — | — | S | — | Y | L |
| I | T | F | E | — | — | S | — | Y | L |
| M | T | F | E | — | — | S | — | Y | L |
| I | I | Y | E | — | — | S | — | Y | L |
| M | I | Y | E | — | — | S | — | Y | L |
| I | T | Y | E | — | — | S | — | Y | L |
| M | T | Y | E | — | — | S | — | Y | L |
| I | I | F | — | — | — | S | — | Y | L |
| M | I | F | — | — | — | S | — | Y | L |
| I | T | F | — | — | — | S | — | Y | L |
| M | T | F | — | — | — | S | — | Y | L |
| I | I | Y | — | — | — | S | — | Y | L |
| M | I | Y | — | — | — | S | — | Y | L |
| I | T | Y | — | — | — | S | — | Y | L |
| M | T | Y | — | — | — | S | — | Y | L |
| I | I | F | E | G | H | Y | — | Y | L |
| M | I | F | E | G | H | Y | — | Y | L |
| I | T | F | E | G | H | Y | — | Y | L |
| M | T | F | E | G | H | Y | — | Y | L |
| I | I | Y | E | G | H | Y | — | Y | L |
| M | I | Y | E | G | H | Y | — | Y | L |
| I | T | Y | E | G | H | Y | — | Y | L |
| M | T | Y | E | G | H | Y | — | Y | L |
| I | I | F | — | G | H | Y | — | Y | L |
| M | I | F | — | G | H | Y | — | Y | L |
| I | T | F | — | G | H | Y | — | Y | L |
| M | T | F | — | G | H | Y | — | Y | L |
| I | I | Y | — | G | H | Y | — | Y | L |
| M | I | Y | — | G | H | Y | — | Y | L |
| I | T | Y | — | G | H | Y | — | Y | L |
| M | T | Y | — | G | H | Y | — | Y | L |
| I | I | F | E | — | H | Y | — | Y | L |
| M | I | F | E | — | H | Y | — | Y | L |
| I | T | F | E | — | H | Y | — | Y | L |
| M | T | F | E | — | H | Y | — | Y | L |
| I | I | Y | E | — | H | Y | — | Y | L |
| M | I | Y | E | — | H | Y | — | Y | L |
| I | T | Y | E | — | H | Y | — | Y | L |
| M | T | Y | E | — | H | Y | — | Y | L |
| I | I | F | — | — | H | Y | — | Y | L |
| M | I | F | — | — | H | Y | — | Y | L |
| I | T | F | — | — | H | Y | — | Y | L |
| M | T | F | — | — | H | Y | — | Y | L |
| I | I | Y | — | — | H | Y | — | Y | L |
| M | I | Y | — | — | H | Y | — | Y | L |
| I | T | Y | — | — | H | Y | — | Y | L |
| M | T | Y | — | — | H | Y | — | Y | L |
| I | I | F | E | G | — | Y | — | Y | L |
| M | I | F | E | G | — | Y | — | Y | L |
| I | T | F | E | G | — | Y | — | Y | L |
| M | T | F | E | G | — | Y | — | Y | L |
| I | I | Y | E | G | — | Y | — | Y | L |
| M | I | Y | E | G | — | Y | — | Y | L |
| I | T | Y | E | G | — | Y | — | Y | L |
| M | T | Y | E | G | — | Y | — | Y | L |
| I | I | F | — | G | — | Y | — | Y | L |
| M | I | F | — | G | — | Y | — | Y | L |
| I | T | F | — | G | — | Y | — | Y | L |
| M | T | F | — | G | — | Y | — | Y | L |
| I | I | Y | — | G | — | Y | — | Y | L |
| M | I | Y | — | G | — | Y | — | Y | L |
| I | T | Y | — | G | — | Y | — | Y | L |
| M | T | Y | — | G | — | Y | — | Y | L |
| I | I | F | E | — | — | Y | — | Y | L |
| M | I | F | E | — | — | Y | — | Y | L |
| I | T | F | E | — | — | Y | — | Y | L |
| M | T | F | E | — | — | Y | — | Y | L |
| I | I | Y | E | — | — | Y | — | Y | L |
| M | I | Y | E | — | — | Y | — | Y | L |
| I | T | Y | E | — | — | Y | — | Y | L |
| M | T | Y | E | — | — | Y | — | Y | L |
| I | I | F | — | — | — | Y | — | Y | L |
| M | I | F | — | — | — | Y | — | Y | L |
| I | T | F | — | — | — | Y | — | Y | L |
| M | T | F | — | — | — | Y | — | Y | L |
| I | I | Y | — | — | — | Y | — | Y | L |
| M | I | Y | — | — | — | Y | — | Y | L |
| I | T | Y | — | — | — | Y | — | Y | L |
| M | T | Y | — | — | — | Y | — | Y | L |
| I | I | F | E | G | H | S | V | G | M |
| M | I | F | E | G | H | S | V | G | M |
| I | T | F | E | G | H | S | V | G | M |
| M | T | F | E | G | H | S | V | G | M |
| I | I | Y | E | G | H | S | V | G | M |
| M | I | Y | E | G | H | S | V | G | M |
| I | T | Y | E | G | H | S | V | G | M |
| M | T | Y | E | G | H | S | V | G | M |
| I | I | F | — | G | H | S | V | G | M |
| M | I | F | — | G | H | S | V | G | M |
| I | T | F | — | G | H | S | V | G | M |
| M | T | F | — | G | H | S | V | G | M |
| I | I | Y | — | G | H | S | V | G | M |
| M | I | Y | — | G | H | S | V | G | M |
| I | T | Y | — | G | H | S | V | G | M |
| M | T | Y | — | G | H | S | V | G | M |
| I | I | F | E | — | H | S | V | G | M |
| M | I | F | E | — | H | S | V | G | M |
| I | T | F | E | — | H | S | V | G | M |
| M | T | F | E | — | H | S | V | G | M |
| I | I | Y | E | — | H | S | V | G | M |
| M | I | Y | E | — | H | S | V | G | M |
| I | T | Y | E | — | H | S | V | G | M |
| M | T | Y | E | — | H | S | V | G | M |
| I | I | F | — | — | H | S | V | G | M |
| M | I | F | — | — | H | S | V | G | M |
| I | T | F | — | — | H | S | V | G | M |
| M | T | F | — | — | H | S | V | G | M |
| I | I | Y | — | — | H | S | V | G | M |
| M | I | Y | — | — | H | S | V | G | M |
| I | T | Y | — | — | H | S | V | G | M |
| M | T | Y | — | — | H | S | V | G | M |
| I | I | F | E | G | — | S | V | G | M |
| M | I | F | E | G | — | S | V | G | M |
| I | T | F | E | G | — | S | V | G | M |
| M | T | F | E | G | — | S | V | G | M |
| I | I | Y | E | G | — | S | V | G | M |
| M | I | Y | E | G | — | S | V | G | M |
| I | T | Y | E | G | — | S | V | G | M |
| M | T | Y | E | G | — | S | V | G | M |
| I | I | F | — | G | — | S | V | G | M |
| M | I | F | — | G | — | S | V | G | M |
| I | T | F | — | G | — | S | V | G | M |
| M | T | F | — | G | — | S | V | G | M |
| I | I | Y | — | G | — | S | V | G | M |
| M | I | Y | — | G | — | S | V | G | M |
| I | T | Y | — | G | — | S | V | G | M |
| M | T | Y | — | G | — | S | V | G | M |
| I | I | F | E | — | — | S | V | G | M |
| M | I | F | E | — | — | S | V | G | M |
| I | T | F | E | — | — | S | V | G | M |
| M | T | F | E | — | — | S | V | G | M |
| I | I | Y | E | — | — | S | V | G | M |
| M | I | Y | E | — | — | S | V | G | M |
| I | T | Y | E | — | — | S | V | G | M |
| M | T | Y | E | — | — | S | V | G | M |
| I | I | F | — | — | — | S | V | G | M |
| M | I | F | — | — | — | S | V | G | M |
| I | T | F | — | — | — | S | V | G | M |
| M | T | F | — | — | — | S | V | G | M |
| I | I | Y | — | — | — | S | V | G | M |
| M | I | Y | — | — | — | S | V | G | M |
| I | T | Y | — | — | — | S | V | G | M |
| M | T | Y | — | — | — | S | V | G | M |
| I | I | F | E | G | H | Y | V | G | M |
| M | I | F | E | G | H | Y | V | G | M |
| I | T | F | E | G | H | Y | V | G | M |
| M | T | F | E | G | H | Y | V | G | M |
| I | I | Y | E | G | H | Y | V | G | M |
| M | I | Y | E | G | H | Y | V | G | M |
| I | T | Y | E | G | H | Y | V | G | M |
| M | T | Y | E | G | H | Y | V | G | M |
| I | I | F | — | G | H | Y | V | G | M |
| M | I | F | — | G | H | Y | V | G | M |
| I | T | F | — | G | H | Y | V | G | M |
| M | T | F | — | G | H | Y | V | G | M |
| I | I | Y | — | G | H | Y | V | G | M |
| M | I | Y | — | G | H | Y | V | G | M |
| I | T | Y | — | G | H | Y | V | G | M |
| M | T | Y | — | G | H | Y | V | G | M |
| I | I | F | E | — | H | Y | V | G | M |
| M | I | F | E | — | H | Y | V | G | M |
| I | T | F | E | — | H | Y | V | G | M |
| M | T | F | E | — | H | Y | V | G | M |
| I | I | Y | E | — | H | Y | V | G | M |
| M | I | Y | E | — | H | Y | V | G | M |
| I | T | Y | E | — | H | Y | V | G | M |
| M | T | Y | E | — | H | Y | V | G | M |
| I | I | F | — | — | H | Y | V | G | M |
| M | I | F | — | — | H | Y | V | G | M |
| I | T | F | — | — | H | Y | V | G | M |
| M | T | F | — | — | H | Y | V | G | M |
| I | I | Y | — | — | H | Y | V | G | M |
| M | I | Y | — | — | H | Y | V | G | M |
| I | T | Y | — | — | H | Y | V | G | M |
| M | T | Y | — | — | H | Y | V | G | M |
| I | I | F | E | G | — | Y | V | G | M |
| M | I | F | E | G | — | Y | V | G | M |
| I | T | F | E | G | — | Y | V | G | M |
| M | T | F | E | G | — | Y | V | G | M |
| I | I | Y | E | G | — | Y | V | G | M |
| M | I | Y | E | G | — | Y | V | G | M |
| I | T | Y | E | G | — | Y | V | G | M |
| M | T | Y | E | G | — | Y | V | G | M |
| I | I | F | — | G | — | Y | V | G | M |
| M | I | F | — | G | — | Y | V | G | M |
| I | T | F | — | G | — | Y | V | G | M |
| M | T | F | — | G | — | Y | V | G | M |
| I | I | Y | — | G | — | Y | V | G | M |
| M | I | Y | — | G | — | Y | V | G | M |
| I | T | Y | — | G | — | Y | V | G | M |
| M | T | Y | — | G | — | Y | V | G | M |
| I | I | F | E | — | — | Y | V | G | M |
| M | I | F | E | — | — | Y | V | G | M |
| I | T | F | E | — | — | Y | V | G | M |
| M | T | F | E | — | — | Y | V | G | M |
| I | I | Y | E | — | — | Y | V | G | M |
| M | I | Y | E | — | — | Y | V | G | M |
| I | T | Y | E | — | — | Y | V | G | M |
| M | T | Y | E | — | — | Y | V | G | M |
| I | I | F | — | — | — | Y | V | G | M |
| M | I | F | — | — | — | Y | V | G | M |
| I | T | F | — | — | — | Y | V | G | M |
| M | T | F | — | — | — | Y | V | G | M |
| I | I | Y | — | — | — | Y | V | G | M |
| M | I | Y | — | — | — | Y | V | G | M |
| I | T | Y | — | — | — | Y | V | G | M |
| M | T | Y | — | — | — | Y | V | G | M |
| I | I | F | E | G | H | S | — | G | M |
| M | I | F | E | G | H | S | — | G | M |
| I | T | F | E | G | H | S | — | G | M |
| M | T | F | E | G | H | S | — | G | M |
| I | I | Y | E | G | H | S | — | G | M |
| M | I | Y | E | G | H | S | — | G | M |
| I | T | Y | E | G | H | S | — | G | M |
| M | T | Y | E | G | H | S | — | G | M |
| I | I | F | — | G | H | S | — | G | M |
| M | I | F | — | G | H | S | — | G | M |
| I | T | F | — | G | H | S | — | G | M |
| M | T | F | — | G | H | S | — | G | M |
| I | I | Y | — | G | H | S | — | G | M |
| M | I | Y | — | G | H | S | — | G | M |
| I | T | Y | — | G | H | S | — | G | M |
| M | T | Y | — | G | H | S | — | G | M |
| I | I | F | E | — | H | S | — | G | M |
| M | I | F | E | — | H | S | — | G | M |
| I | T | F | E | — | H | S | — | G | M |
| M | T | F | E | — | H | S | — | G | M |
| I | I | Y | E | — | H | S | — | G | M |
| M | I | Y | E | — | H | S | — | G | M |
| I | T | Y | E | — | H | S | — | G | M |
| M | T | Y | E | — | H | S | — | G | M |
| I | I | F | — | — | H | S | — | G | M |
| M | I | F | — | — | H | S | — | G | M |
| I | T | F | — | — | H | S | — | G | M |
| M | T | F | — | — | H | S | — | G | M |
| I | I | Y | — | — | H | S | — | G | M |
| M | I | Y | — | — | H | S | — | G | M |
| I | T | Y | — | — | H | S | — | G | M |
| M | T | Y | — | — | H | S | — | G | M |
| I | I | F | E | G | — | S | — | G | M |
| M | I | F | E | G | — | S | — | G | M |
| I | T | F | E | G | — | S | — | G | M |
| M | T | F | E | G | — | S | — | G | M |
| I | I | Y | E | G | — | S | — | G | M |
| M | I | Y | E | G | — | S | — | G | M |
| I | T | Y | E | G | — | S | — | G | M |
| M | T | Y | E | G | — | S | — | G | M |
| I | I | F | — | G | — | S | — | G | M |
| M | I | F | — | G | — | S | — | G | M |
| I | T | F | — | G | — | S | — | G | M |
| M | T | F | — | G | — | S | — | G | M |
| I | I | Y | — | G | — | S | — | G | M |
| M | I | Y | — | G | — | S | — | G | M |
| I | T | Y | — | G | — | S | — | G | M |
| M | T | Y | — | G | — | S | — | G | M |
| I | I | F | E | — | — | S | — | G | M |
| M | I | F | E | — | — | S | — | G | M |
| I | T | F | E | — | — | S | — | G | M |
| M | T | F | E | — | — | S | — | G | M |
| I | I | Y | E | — | — | S | — | G | M |
| M | I | Y | E | — | — | S | — | G | M |
| I | T | Y | E | — | — | S | — | G | M |
| M | T | Y | E | — | — | S | — | G | M |
| I | I | F | — | — | — | S | — | G | M |
| M | I | F | — | — | — | S | — | G | M |
| I | T | F | — | — | — | S | — | G | M |
| M | T | F | — | — | — | S | — | G | M |
| I | I | Y | — | — | — | S | — | G | M |
| M | I | Y | — | — | — | S | — | G | M |
| I | T | Y | — | — | — | S | — | G | M |
| M | T | Y | — | — | — | S | — | G | M |
| I | I | F | E | G | H | Y | — | G | M |
| M | I | F | E | G | H | Y | — | G | M |
| I | T | F | E | G | H | Y | — | G | M |
| M | T | F | E | G | H | Y | — | G | M |
| I | I | Y | E | G | H | Y | — | G | M |
| M | I | Y | E | G | H | Y | — | G | M |
| I | T | Y | E | G | H | Y | — | G | M |
| M | T | Y | E | G | H | Y | — | G | M |
| I | I | F | — | G | H | Y | — | G | M |
| M | I | F | — | G | H | Y | — | G | M |
| I | T | F | — | G | H | Y | — | G | M |
| M | T | F | — | G | H | Y | — | G | M |
| I | I | Y | — | G | H | Y | — | G | M |
| M | I | Y | — | G | H | Y | — | G | M |
| I | T | Y | — | G | H | Y | — | G | M |
| M | T | Y | — | G | H | Y | — | G | M |
| I | I | F | E | — | H | Y | — | G | M |
| M | I | F | E | — | H | Y | — | G | M |
| I | T | F | E | — | H | Y | — | G | M |
| M | T | F | E | — | H | Y | — | G | M |
| I | I | Y | E | — | H | Y | — | G | M |
| M | I | Y | E | — | H | Y | — | G | M |
| I | T | Y | E | — | H | Y | — | G | M |
| M | T | Y | E | — | H | Y | — | G | M |
| I | I | F | — | — | H | Y | — | G | M |
| M | I | F | — | — | H | Y | — | G | M |
| I | T | F | — | — | H | Y | — | G | M |
| M | T | F | — | — | H | Y | — | G | M |
| I | I | Y | — | — | H | Y | — | G | M |
| M | I | Y | — | — | H | Y | — | G | M |
| I | T | Y | — | — | H | Y | — | G | M |
| M | T | Y | — | — | H | Y | — | G | M |
| I | I | F | E | G | — | Y | — | G | M |
| M | I | F | E | G | — | Y | — | G | M |
| I | T | F | E | G | — | Y | — | G | M |
| M | T | F | E | G | — | Y | — | G | M |
| I | I | Y | E | G | — | Y | — | G | M |
| M | I | Y | E | G | — | Y | — | G | M |
| I | T | Y | E | G | — | Y | — | G | M |
| M | T | Y | E | G | — | Y | — | G | M |
| I | I | F | — | G | — | Y | — | G | M |
| M | I | F | — | G | — | Y | — | G | M |
| I | T | F | — | G | — | Y | — | G | M |
| M | T | F | — | G | — | Y | — | G | M |
| I | I | Y | — | G | — | Y | — | G | M |
| M | I | Y | — | G | — | Y | — | G | M |
| I | T | Y | — | G | — | Y | — | G | M |
| M | T | Y | — | G | — | Y | — | G | M |
| I | I | F | E | — | — | Y | — | G | M |
| M | I | F | E | — | — | Y | — | G | M |
| I | T | F | E | — | — | Y | — | G | M |
| M | T | F | E | — | — | Y | — | G | M |
| I | I | Y | E | — | — | Y | — | G | M |
| M | I | Y | E | — | — | Y | — | G | M |
| I | T | Y | E | — | — | Y | — | G | M |
| M | T | Y | E | — | — | Y | — | G | M |
| I | I | F | — | — | — | Y | — | G | M |
| M | I | F | — | — | — | Y | — | G | M |
| I | T | F | — | — | — | Y | — | G | M |
| M | T | F | — | — | — | Y | — | G | M |
| I | I | Y | — | — | — | Y | — | G | M |
| M | I | Y | — | — | — | Y | — | G | M |
| I | T | Y | — | — | — | Y | — | G | M |
| M | T | Y | — | — | — | Y | — | G | M |
| I | I | F | E | G | H | S | V | Y | M |
| M | I | F | E | G | H | S | V | Y | M |
| I | T | F | E | G | H | S | V | Y | M |
| M | T | F | E | G | H | S | V | Y | M |
| I | I | Y | E | G | H | S | V | Y | M |
| M | I | Y | E | G | H | S | V | Y | M |
| I | T | Y | E | G | H | S | V | Y | M |
| M | T | Y | E | G | H | S | V | Y | M |
| I | I | F | — | G | H | S | V | Y | M |
| M | I | F | — | G | H | S | V | Y | M |
| I | T | F | — | G | H | S | V | Y | M |
| M | T | F | — | G | H | S | V | Y | M |
| I | I | Y | — | G | H | S | V | Y | M |
| M | I | Y | — | G | H | S | V | Y | M |
| I | T | Y | — | G | H | S | V | Y | M |
| M | T | Y | — | G | H | S | V | Y | M |
| I | I | F | E | — | H | S | V | Y | M |
| M | I | F | E | — | H | S | V | Y | M |
| I | T | F | E | — | H | S | V | Y | M |
| M | T | F | E | — | H | S | V | Y | M |
| I | I | Y | E | — | H | S | V | Y | M |
| M | I | Y | E | — | H | S | V | Y | M |
| I | T | Y | E | — | H | S | V | Y | M |
| M | T | Y | E | — | H | S | V | Y | M |
| I | I | F | — | — | H | S | V | Y | M |
| M | I | F | — | — | H | S | V | Y | M |
| I | T | F | — | — | H | S | V | Y | M |
| M | T | F | — | — | H | S | V | Y | M |
| I | I | Y | — | — | H | S | V | Y | M |
| M | I | Y | — | — | H | S | V | Y | M |
| I | T | Y | — | — | H | S | V | Y | M |
| M | T | Y | — | — | H | S | V | Y | M |
| I | I | F | E | G | — | S | V | Y | M |
| M | I | F | E | G | — | S | V | Y | M |
| I | T | F | E | G | — | S | V | Y | M |
| M | T | F | E | G | — | S | V | Y | M |
| I | I | Y | E | G | — | S | V | Y | M |
| M | I | Y | E | G | — | S | V | Y | M |
| I | T | Y | E | G | — | S | V | Y | M |
| M | T | Y | E | G | — | S | V | Y | M |
| I | I | F | — | G | — | S | V | Y | M |
| M | I | F | — | G | — | S | V | Y | M |
| I | T | F | — | G | — | S | V | Y | M |
| M | T | F | — | G | — | S | V | Y | M |
| I | I | Y | — | G | — | S | V | Y | M |
| M | I | Y | — | G | — | S | V | Y | M |
| I | T | Y | — | G | — | S | V | Y | M |
| M | T | Y | — | G | — | S | V | Y | M |
| I | I | F | E | — | — | S | V | Y | M |
| M | I | F | E | — | — | S | V | Y | M |
| I | T | F | E | — | — | S | V | Y | M |
| M | T | F | E | — | — | S | V | Y | M |
| I | I | Y | E | — | — | S | V | Y | M |
| M | I | Y | E | — | — | S | V | Y | M |
| I | T | Y | E | — | — | S | V | Y | M |
| M | T | Y | E | — | — | S | V | Y | M |
| I | I | F | — | — | — | S | V | Y | M |
| M | I | F | — | — | — | S | V | Y | M |
| I | T | F | — | — | — | S | V | Y | M |
| M | T | F | — | — | — | S | V | Y | M |
| I | I | Y | — | — | — | S | V | Y | M |
| M | I | Y | — | — | — | S | V | Y | M |
| I | T | Y | — | — | — | S | V | Y | M |
| M | T | Y | — | — | — | S | V | Y | M |
| I | I | F | E | G | H | Y | V | Y | M |
| M | I | F | E | G | H | Y | V | Y | M |
| I | T | F | E | G | H | Y | V | Y | M |
| M | T | F | E | G | H | Y | V | Y | M |
| I | I | Y | E | G | H | Y | V | Y | M |
| M | I | Y | E | G | H | Y | V | Y | M |
| I | T | Y | E | G | H | Y | V | Y | M |
| M | T | Y | E | G | H | Y | V | Y | M |
| I | I | F | — | G | H | Y | V | Y | M |
| M | I | F | — | G | H | Y | V | Y | M |
| I | T | F | — | G | H | Y | V | Y | M |
| M | T | F | — | G | H | Y | V | Y | M |
| I | I | Y | — | G | H | Y | V | Y | M |
| M | I | Y | — | G | H | Y | V | Y | M |
| I | T | Y | — | G | H | Y | V | Y | M |
| M | T | Y | — | G | H | Y | V | Y | M |
| I | I | F | E | — | H | Y | V | Y | M |
| M | I | F | E | — | H | Y | V | Y | M |
| I | T | F | E | — | H | Y | V | Y | M |
| M | T | F | E | — | H | Y | V | Y | M |
| I | I | Y | E | — | H | Y | V | Y | M |
| M | I | Y | E | — | H | Y | V | Y | M |
| I | T | Y | E | — | H | Y | V | Y | M |
| M | T | Y | E | — | H | Y | V | Y | M |
| I | I | F | — | — | H | Y | V | Y | M |
| M | I | F | — | — | H | Y | V | Y | M |
| I | T | F | — | — | H | Y | V | Y | M |
| M | T | F | — | — | H | Y | V | Y | M |
| I | I | Y | — | — | H | Y | V | Y | M |
| M | I | Y | — | — | H | Y | V | Y | M |
| I | T | Y | — | — | H | Y | V | Y | M |
| M | T | Y | — | — | H | Y | V | Y | M |
| I | I | F | E | G | — | Y | V | Y | M |
| M | I | F | E | G | — | Y | V | Y | M |
| I | T | F | E | G | — | Y | V | Y | M |
| M | T | F | E | G | — | Y | V | Y | M |
| I | I | Y | E | G | — | Y | V | Y | M |
| M | I | Y | E | G | — | Y | V | Y | M |
| I | T | Y | E | G | — | Y | V | Y | M |
| M | T | Y | E | G | — | Y | V | Y | M |
| I | I | F | — | G | — | Y | V | Y | M |
| M | I | F | — | G | — | Y | V | Y | M |
| I | T | F | — | G | — | Y | V | Y | M |
| M | T | F | — | G | — | Y | V | Y | M |
| I | I | Y | — | G | — | Y | V | Y | M |
| M | I | Y | — | G | — | Y | V | Y | M |
| I | T | Y | — | G | — | Y | V | Y | M |
| M | T | Y | — | G | — | Y | V | Y | M |
| I | I | F | E | — | — | Y | V | Y | M |
| M | I | F | E | — | — | Y | V | Y | M |
| I | T | F | E | — | — | Y | V | Y | M |
| M | T | F | E | — | — | Y | V | Y | M |
| I | I | Y | E | — | — | Y | V | Y | M |
| M | I | Y | E | — | — | Y | V | Y | M |
| I | T | Y | E | — | — | Y | V | Y | M |
| M | T | Y | E | — | — | Y | V | Y | M |
| I | I | F | — | — | — | Y | V | Y | M |
| M | I | F | — | — | — | Y | V | Y | M |
| I | T | F | — | — | — | Y | V | Y | M |
| M | T | F | — | — | — | Y | V | Y | M |
| I | I | Y | — | — | — | Y | V | Y | M |
| M | I | Y | — | — | — | Y | V | Y | M |
| I | T | Y | — | — | — | Y | V | Y | M |
| M | T | Y | — | — | — | Y | V | Y | M |
| I | I | F | E | G | H | S | — | Y | M |
| M | I | F | E | G | H | S | — | Y | M |
| I | T | F | E | G | H | S | — | Y | M |
| M | T | F | E | G | H | S | — | Y | M |
| I | I | Y | E | G | H | S | — | Y | M |
| M | I | Y | E | G | H | S | — | Y | M |
| I | T | Y | E | G | H | S | — | Y | M |
| M | T | Y | E | G | H | S | — | Y | M |
| I | I | F | — | G | H | S | — | Y | M |
| M | I | F | — | G | H | S | — | Y | M |
| I | T | F | — | G | H | S | — | Y | M |
| M | T | F | — | G | H | S | — | Y | M |
| I | I | Y | — | G | H | S | — | Y | M |
| M | I | Y | — | G | H | S | — | Y | M |
| I | T | Y | — | G | H | S | — | Y | M |
| M | T | Y | — | G | H | S | — | Y | M |
| I | I | F | E | — | H | S | — | Y | M |
| M | I | F | E | — | H | S | — | Y | M |
| I | T | F | E | — | H | S | — | Y | M |
| M | T | F | E | — | H | S | — | Y | M |
| I | I | Y | E | — | H | S | — | Y | M |
| M | I | Y | E | — | H | S | — | Y | M |
| I | T | Y | E | — | H | S | — | Y | M |
| M | T | Y | E | — | H | S | — | Y | M |
| I | I | F | — | — | H | S | — | Y | M |
| M | I | F | — | — | H | S | — | Y | M |
| I | T | F | — | — | H | S | — | Y | M |
| M | T | F | — | — | H | S | — | Y | M |
| I | I | Y | — | — | H | S | — | Y | M |
| M | I | Y | — | — | H | S | — | Y | M |
| I | T | Y | — | — | H | S | — | Y | M |
| M | T | Y | — | — | H | S | — | Y | M |
| I | I | F | E | G | — | S | — | Y | M |
| M | I | F | E | G | — | S | — | Y | M |
| I | T | F | E | G | — | S | — | Y | M |
| M | T | F | E | G | — | S | — | Y | M |
| I | I | Y | E | G | — | S | — | Y | M |
| M | I | Y | E | G | — | S | — | Y | M |
| I | T | Y | E | G | — | S | — | Y | M |
| M | T | Y | E | G | — | S | — | Y | M |
| I | I | F | — | G | — | S | — | Y | M |
| M | I | F | — | G | — | S | — | Y | M |
| I | T | F | — | G | — | S | — | Y | M |
| M | T | F | — | G | — | S | — | Y | M |
| I | I | Y | — | G | — | S | — | Y | M |
| M | I | Y | — | G | — | S | — | Y | M |
| I | T | Y | — | G | — | S | — | Y | M |
| M | T | Y | — | G | — | S | — | Y | M |
| I | I | F | E | — | — | S | — | Y | M |
| M | I | F | E | — | — | S | — | Y | M |
| I | T | F | E | — | — | S | — | Y | M |
| M | T | F | E | — | — | S | — | Y | M |
| I | I | Y | E | — | — | S | — | Y | M |
| M | I | Y | E | — | — | S | — | Y | M |
| I | T | Y | E | — | — | S | — | Y | M |
| M | T | Y | E | — | — | S | — | Y | M |
| I | I | F | — | — | — | S | — | Y | M |
| M | I | F | — | — | — | S | — | Y | M |
| I | T | F | — | — | — | S | — | Y | M |
| M | T | F | — | — | — | S | — | Y | M |
| I | I | Y | — | — | — | S | — | Y | M |
| M | I | Y | — | — | — | S | — | Y | M |
| I | T | Y | — | — | — | S | — | Y | M |
| M | T | Y | — | — | — | S | — | Y | M |
| I | I | F | E | G | H | Y | — | Y | M |
| M | I | F | E | G | H | Y | — | Y | M |
| I | T | F | E | G | H | Y | — | Y | M |
| M | T | F | E | G | H | Y | — | Y | M |
| I | I | Y | E | G | H | Y | — | Y | M |
| M | I | Y | E | G | H | Y | — | Y | M |
| I | T | Y | E | G | H | Y | — | Y | M |
| M | T | Y | E | G | H | Y | — | Y | M |
| I | I | F | — | G | H | Y | — | Y | M |
| M | I | F | — | G | H | Y | — | Y | M |
| I | T | F | — | G | H | Y | — | Y | M |
| M | T | F | — | G | H | Y | — | Y | M |
| I | I | Y | — | G | H | Y | — | Y | M |
| M | I | Y | — | G | H | Y | — | Y | M |
| I | T | Y | — | G | H | Y | — | Y | M |
| M | T | Y | — | G | H | Y | — | Y | M |
| I | I | F | E | — | H | Y | — | Y | M |
| M | I | F | E | — | H | Y | — | Y | M |
| I | T | F | E | — | H | Y | — | Y | M |
| M | T | F | E | — | H | Y | — | Y | M |
| I | I | Y | E | — | H | Y | — | Y | M |
| M | I | Y | E | — | H | Y | — | Y | M |
| I | T | Y | E | — | H | Y | — | Y | M |
| M | T | Y | E | — | H | Y | — | Y | M |
| I | I | F | — | — | H | Y | — | Y | M |
| M | I | F | — | — | H | Y | — | Y | M |
| I | T | F | — | — | H | Y | — | Y | M |
| M | T | F | — | — | H | Y | — | Y | M |
| I | I | Y | — | — | H | Y | — | Y | M |
| M | I | Y | — | — | H | Y | — | Y | M |
| I | T | Y | — | — | H | Y | — | Y | M |
| M | T | Y | — | — | H | Y | — | Y | M |
| I | I | F | E | G | — | Y | — | Y | M |
| M | I | F | E | G | — | Y | — | Y | M |
| I | T | F | E | G | — | Y | — | Y | M |
| M | T | F | E | G | — | Y | — | Y | M |
| I | I | Y | E | G | — | Y | — | Y | M |
| M | I | Y | E | G | — | Y | — | Y | M |
| I | T | Y | E | G | — | Y | — | Y | M |
| M | T | Y | E | G | — | Y | — | Y | M |
| I | I | F | — | G | — | Y | — | Y | M |
| M | I | F | — | G | — | Y | — | Y | M |
| I | T | F | — | G | — | Y | — | Y | M |
| M | T | F | — | G | — | Y | — | Y | M |
| I | I | Y | — | G | — | Y | — | Y | M |
| M | I | Y | — | G | — | Y | — | Y | M |
| I | T | Y | — | G | — | Y | — | Y | M |
| M | T | Y | — | G | — | Y | — | Y | M |
| I | I | F | E | — | — | Y | — | Y | M |
| M | I | F | E | — | — | Y | — | Y | M |
| I | T | F | E | — | — | Y | — | Y | M |
| M | T | F | E | — | — | Y | — | Y | M |
| I | I | Y | E | — | — | Y | — | Y | M |
| M | I | Y | E | — | — | Y | — | Y | M |
| I | T | Y | E | — | — | Y | — | Y | M |
| M | T | Y | E | — | — | Y | — | Y | M |
| I | I | F | — | — | — | Y | — | Y | M |
| M | I | F | — | — | — | Y | — | Y | M |
| I | T | F | — | — | — | Y | — | Y | M |
| M | T | F | — | — | — | Y | — | Y | M |
| I | I | Y | — | — | — | Y | — | Y | M |
| M | I | Y | — | — | — | Y | — | Y | M |
| I | T | Y | — | — | — | Y | — | Y | M |
| M | T | Y | — | — | — | Y | — | Y | M |

“—” indicates the absence of a residue at the position with reference to SEQ ID NO: 2

TABLE 8
Exemplary Mutations of mAB 10E9.1 Light Chain (SEQ ID NO: 4) to
Germline at the Indicated Residue Number

| 28 | 30 | 32 | 43 | 85 | 90 | 91 | 94 | 107 |
|---|---|---|---|---|---|---|---|---|
| D | R | F | L | S | S | H | V | R |
| G | R | F | L | S | S | H | V | R |
| D | S | F | L | S | S | H | V | R |
| G | S | F | L | S | S | H | V | R |
| D | R | Y | L | S | S | H | V | R |
| G | R | Y | L | S | S | H | V | R |
| D | S | Y | L | S | S | H | V | R |
| G | S | Y | L | S | S | H | V | R |
| D | R | F | V | S | S | H | V | R |
| G | R | F | V | S | S | H | V | R |
| D | S | F | V | S | S | H | V | R |
| G | S | F | V | S | S | H | V | R |
| D | R | Y | V | S | S | H | V | R |
| G | R | Y | V | S | S | H | V | R |
| D | S | Y | V | S | S | H | V | R |
| G | S | Y | V | S | S | H | V | R |
| D | R | F | L | T | S | H | V | R |
| G | R | F | L | T | S | H | V | R |
| D | S | F | L | T | S | H | V | R |
| G | S | F | L | T | S | H | V | R |
| D | R | Y | L | T | S | H | V | R |
| G | R | Y | L | T | S | H | V | R |
| D | S | Y | L | T | S | H | V | R |
| G | S | Y | L | T | S | H | V | R |
| D | R | F | V | T | S | H | V | R |
| G | R | F | V | T | S | H | V | R |
| D | S | F | V | T | S | H | V | R |
| G | S | F | V | T | S | H | V | R |
| D | R | Y | V | T | S | H | V | R |
| G | R | Y | V | T | S | H | V | R |
| D | S | Y | V | T | S | H | V | R |
| G | S | Y | V | T | S | H | V | R |
| D | R | F | L | S | K | H | V | R |
| G | R | F | L | S | K | H | V | R |
| D | S | F | L | S | K | H | V | R |
| G | S | F | L | S | K | H | V | R |
| D | R | Y | L | S | K | H | V | R |
| G | R | Y | L | S | K | H | V | R |
| D | S | Y | L | S | K | H | V | R |
| G | S | Y | L | S | K | H | V | R |
| D | R | F | V | S | K | H | V | R |
| G | R | F | V | S | K | H | V | R |
| D | S | F | V | S | K | H | V | R |
| G | S | F | V | S | K | H | V | R |
| D | R | Y | V | S | K | H | V | R |
| G | R | Y | V | S | K | H | V | R |
| D | S | Y | V | S | K | H | V | R |
| G | S | Y | V | S | K | H | V | R |
| D | R | F | L | T | K | H | V | R |
| G | R | F | L | T | K | H | V | R |
| D | S | F | L | T | K | H | V | R |
| G | S | F | L | T | K | H | V | R |
| D | R | Y | L | T | K | H | V | R |
| G | R | Y | L | T | K | H | V | R |
| D | S | Y | L | T | K | H | V | R |
| G | S | Y | L | T | K | H | V | R |
| D | R | F | V | T | K | H | V | R |
| G | R | F | V | T | K | H | V | R |
| D | S | F | V | T | K | H | V | R |
| G | S | F | V | T | K | H | V | R |
| D | R | Y | V | T | K | H | V | R |
| G | R | Y | V | T | K | H | V | R |
| D | S | Y | V | T | K | H | V | R |
| G | S | Y | V | T | K | H | V | R |
| D | R | F | L | S | S | Y | V | R |
| G | R | F | L | S | S | Y | V | R |
| D | S | F | L | S | S | Y | V | R |
| G | S | F | L | S | S | Y | V | R |
| D | R | Y | L | S | S | Y | V | R |
| G | R | Y | L | S | S | Y | V | R |
| D | S | Y | L | S | S | Y | V | R |
| G | S | Y | L | S | S | Y | V | R |
| D | R | F | V | S | S | Y | V | R |
| G | R | F | V | S | S | Y | V | R |
| D | S | F | V | S | S | Y | V | R |
| G | S | F | V | S | S | Y | V | R |
| D | R | Y | V | S | S | Y | V | R |
| G | R | Y | V | S | S | Y | V | R |
| D | S | Y | V | S | S | Y | V | R |
| G | S | Y | V | S | S | Y | V | R |
| D | R | F | L | T | S | Y | V | R |
| G | R | F | L | T | S | Y | V | R |
| D | S | F | L | T | S | Y | V | R |
| G | S | F | L | T | S | Y | V | R |
| D | R | Y | L | T | S | Y | V | R |
| G | R | Y | L | T | S | Y | V | R |
| D | S | Y | L | T | S | Y | V | R |
| G | S | Y | L | T | S | Y | V | R |
| D | R | F | V | T | S | Y | V | R |
| G | R | F | V | T | S | Y | V | R |
| D | S | F | V | T | S | Y | V | R |
| G | S | F | V | T | S | Y | V | R |
| D | R | Y | V | T | S | Y | V | R |
| G | R | Y | V | T | S | Y | V | R |
| D | S | Y | V | T | S | Y | V | R |
| G | S | Y | V | T | S | Y | V | R |
| D | R | F | L | S | K | Y | V | R |
| G | R | F | L | S | K | Y | V | R |
| D | S | F | L | S | K | Y | V | R |
| G | S | F | L | S | K | Y | V | R |
| D | R | Y | L | S | K | Y | V | R |
| G | R | Y | L | S | K | Y | V | R |
| D | S | Y | L | S | K | Y | V | R |
| G | S | Y | L | S | K | Y | V | R |
| D | R | F | V | S | K | Y | V | R |
| G | R | F | V | S | K | Y | V | R |
| D | S | F | V | S | K | Y | V | R |
| G | S | F | V | S | K | Y | V | R |
| D | R | Y | V | S | K | Y | V | R |
| G | R | Y | V | S | K | Y | V | R |
| D | S | Y | V | S | K | Y | V | R |
| G | S | Y | V | S | K | Y | V | R |
| D | R | F | L | T | K | Y | V | R |
| G | R | F | L | T | K | Y | V | R |
| D | S | F | L | T | K | Y | V | R |
| G | S | F | L | T | K | Y | V | R |
| D | R | Y | L | T | K | Y | V | R |
| G | R | Y | L | T | K | Y | V | R |
| D | S | Y | L | T | K | Y | V | R |
| G | S | Y | L | T | K | Y | V | R |
| D | R | F | V | T | K | Y | V | R |
| G | R | F | V | T | K | Y | V | R |
| D | S | F | V | T | K | Y | V | R |
| G | S | F | V | T | K | Y | V | R |
| D | R | Y | V | T | K | Y | V | R |
| G | R | Y | V | T | K | Y | V | R |
| D | S | Y | V | T | K | Y | V | R |
| G | S | Y | V | T | K | Y | V | R |
| D | R | F | L | S | S | H | A | R |
| G | R | F | L | S | S | H | A | R |
| D | S | F | L | S | S | H | A | R |
| G | S | F | L | S | S | H | A | R |
| D | R | Y | L | S | S | H | A | R |
| G | R | Y | L | S | S | H | A | R |
| D | S | Y | L | S | S | H | A | R |
| G | S | Y | L | S | S | H | A | R |
| D | R | F | V | S | S | H | A | R |
| G | R | F | V | S | S | H | A | R |
| D | S | F | V | S | S | H | A | R |
| G | S | F | V | S | S | H | A | R |
| D | R | Y | V | S | S | H | A | R |
| G | R | Y | V | S | S | H | A | R |
| D | S | Y | V | S | S | H | A | R |
| G | S | Y | V | S | S | H | A | R |
| D | R | F | L | T | S | H | A | R |
| G | R | F | L | T | S | H | A | R |
| D | S | F | L | T | S | H | A | R |
| G | S | F | L | T | S | H | A | R |
| D | R | Y | L | T | S | H | A | R |
| G | R | Y | L | T | S | H | A | R |
| D | S | Y | L | T | S | H | A | R |
| G | S | Y | L | T | S | H | A | R |
| D | R | F | V | T | S | H | A | R |
| G | R | F | V | T | S | H | A | R |
| D | S | F | V | T | S | H | A | R |
| G | S | F | V | T | S | H | A | R |
| D | R | Y | V | T | S | H | A | R |
| G | R | Y | V | T | S | H | A | R |
| D | S | Y | V | T | S | H | A | R |
| G | S | Y | V | T | S | H | A | R |
| D | R | F | L | S | K | H | A | R |
| G | R | F | L | S | K | H | A | R |
| D | S | F | L | S | K | H | A | R |
| G | S | F | L | S | K | H | A | R |
| D | R | Y | L | S | K | H | A | R |
| G | R | Y | L | S | K | H | A | R |
| D | S | Y | L | S | K | H | A | R |
| G | S | Y | L | S | K | H | A | R |
| D | R | F | V | S | K | H | A | R |
| G | R | F | V | S | K | H | A | R |
| D | S | F | V | S | K | H | A | R |
| G | S | F | V | S | K | H | A | R |
| D | R | Y | V | S | K | H | A | R |
| G | R | Y | V | S | K | H | A | R |
| D | S | Y | V | S | K | H | A | R |
| G | S | Y | V | S | K | H | A | R |
| D | R | F | L | T | K | H | A | R |
| G | R | F | L | T | K | H | A | R |
| D | S | F | L | T | K | H | A | R |
| G | S | F | L | T | K | H | A | R |
| D | R | Y | L | T | K | H | A | R |
| G | R | Y | L | T | K | H | A | R |
| D | S | Y | L | T | K | H | A | R |
| G | S | Y | L | T | K | H | A | R |
| D | R | F | V | T | K | H | A | R |
| G | R | F | V | T | K | H | A | R |
| D | S | F | V | T | K | H | A | R |
| G | S | F | V | T | K | H | A | R |
| D | R | Y | V | T | K | H | A | R |
| G | R | Y | V | T | K | H | A | R |
| D | S | Y | V | T | K | H | A | R |
| G | S | Y | V | T | K | H | A | R |
| D | R | F | L | S | S | Y | A | R |
| G | R | F | L | S | S | Y | A | R |
| D | S | F | L | S | S | Y | A | R |
| G | S | F | L | S | S | Y | A | R |
| D | R | Y | L | S | S | Y | A | R |
| G | R | Y | L | S | S | Y | A | R |
| D | S | Y | L | S | S | Y | A | R |
| G | S | Y | L | S | S | Y | A | R |
| D | R | F | V | S | S | Y | A | R |
| G | R | F | V | S | S | Y | A | R |
| D | S | F | V | S | S | Y | A | R |
| G | S | F | V | S | S | Y | A | R |
| D | R | Y | V | S | S | Y | A | R |
| G | R | Y | V | S | S | Y | A | R |
| D | S | Y | V | S | S | Y | A | R |
| G | S | Y | V | S | S | Y | A | R |
| D | R | F | L | T | S | Y | A | R |
| G | R | F | L | T | S | Y | A | R |
| D | S | F | L | T | S | Y | A | R |
| G | S | F | L | T | S | Y | A | R |
| D | R | Y | L | T | S | Y | A | R |
| G | R | Y | L | T | S | Y | A | R |
| D | S | Y | L | T | S | Y | A | R |
| G | S | Y | L | T | S | Y | A | R |
| D | R | F | V | T | S | Y | A | R |
| G | R | F | V | T | S | Y | A | R |
| D | S | F | V | T | S | Y | A | R |
| G | S | F | V | T | S | Y | A | R |
| D | R | Y | V | T | S | Y | A | R |
| G | R | Y | V | T | S | Y | A | R |
| D | S | Y | V | T | S | Y | A | R |
| G | S | Y | V | T | S | Y | A | R |
| D | R | F | L | S | K | Y | A | R |
| G | R | F | L | S | K | Y | A | R |
| D | S | F | L | S | K | Y | A | R |
| G | S | F | L | S | K | Y | A | R |
| D | R | Y | L | S | K | Y | A | R |
| G | R | Y | L | S | K | Y | A | R |
| D | S | Y | L | S | K | Y | A | R |
| G | S | Y | L | S | K | Y | A | R |
| D | R | F | V | S | K | Y | A | R |
| G | R | F | V | S | K | Y | A | R |
| D | S | F | V | S | K | Y | A | R |
| G | S | F | V | S | K | Y | A | R |
| D | R | Y | V | S | K | Y | A | R |
| G | R | Y | V | S | K | Y | A | R |
| D | S | Y | V | S | K | Y | A | R |
| G | S | Y | V | S | K | Y | A | R |
| D | R | F | L | T | K | Y | A | R |
| G | R | F | L | T | K | Y | A | R |
| D | S | F | L | T | K | Y | A | R |
| G | S | F | L | T | K | Y | A | R |
| D | R | Y | L | T | K | Y | A | R |
| G | R | Y | L | T | K | Y | A | R |
| D | S | Y | L | T | K | Y | A | R |
| G | S | Y | L | T | K | Y | A | R |
| D | R | F | V | T | K | Y | A | R |
| G | R | F | V | T | K | Y | A | R |
| D | S | F | V | T | K | Y | A | R |
| G | S | F | V | T | K | Y | A | R |
| D | R | Y | V | T | K | Y | A | R |
| G | R | Y | V | T | K | Y | A | R |
| D | S | Y | V | T | K | Y | A | R |
| G | S | Y | V | T | K | Y | A | R |
| D | R | F | L | S | S | H | V | K |
| G | R | F | L | S | S | H | V | K |
| D | S | F | L | S | S | H | V | K |
| G | S | F | L | S | S | H | V | K |
| D | R | Y | L | S | S | H | V | K |
| G | R | Y | L | S | S | H | V | K |
| D | S | Y | L | S | S | H | V | K |
| G | S | Y | L | S | S | H | V | K |
| D | R | F | V | S | S | H | V | K |
| G | R | F | V | S | S | H | V | K |
| D | S | F | V | S | S | H | V | K |
| G | S | F | V | S | S | H | V | K |
| D | R | Y | V | S | S | H | V | K |
| G | R | Y | V | S | S | H | V | K |
| D | S | Y | V | S | S | H | V | K |
| G | S | Y | V | S | S | H | V | K |
| D | R | F | L | T | S | H | V | K |
| G | R | F | L | T | S | H | V | K |
| D | S | F | L | T | S | H | V | K |
| G | S | F | L | T | S | H | V | K |
| D | R | Y | L | T | S | H | V | K |
| G | R | Y | L | T | S | H | V | K |
| D | S | Y | L | T | S | H | V | K |
| G | S | Y | L | T | S | H | V | K |
| D | R | F | V | T | S | H | V | K |
| G | R | F | V | T | S | H | V | K |
| D | S | F | V | T | S | H | V | K |
| G | S | F | V | T | S | H | V | K |
| D | R | Y | V | T | S | H | V | K |
| G | R | Y | V | T | S | H | V | K |
| D | S | Y | V | S | S | H | V | K |
| G | S | Y | V | T | S | H | V | K |
| D | R | F | L | S | K | H | V | K |
| G | R | F | L | S | K | H | V | K |
| D | S | F | L | S | K | H | V | K |
| G | S | F | L | S | K | H | V | K |
| D | R | Y | L | S | K | H | V | K |
| G | R | Y | L | S | K | H | V | K |
| D | S | Y | L | S | K | H | V | K |
| G | S | Y | L | S | K | H | V | K |
| D | R | F | V | S | K | H | V | K |
| G | R | F | V | S | K | H | V | K |
| D | S | F | V | S | K | H | V | K |
| G | S | F | V | S | K | H | V | K |
| D | R | Y | V | S | K | H | V | K |
| G | R | Y | V | S | K | H | V | K |
| D | S | Y | V | S | K | H | V | K |
| G | S | Y | V | S | K | H | V | K |
| D | R | F | L | T | K | H | V | K |
| G | R | F | L | T | K | H | V | K |
| D | S | F | L | T | K | H | V | K |
| G | S | F | L | T | K | H | V | K |
| D | R | Y | L | T | K | H | V | K |
| G | R | Y | L | T | K | H | V | K |
| D | S | Y | L | T | K | H | V | K |
| G | S | Y | L | T | K | H | V | K |
| D | R | F | V | T | K | H | V | K |
| G | R | F | V | T | K | H | V | K |
| D | S | F | V | T | K | H | V | K |
| G | S | F | V | T | K | H | V | K |
| D | R | Y | V | T | K | H | V | K |
| G | R | Y | V | T | K | H | V | K |
| D | S | Y | V | T | K | H | V | K |
| G | S | Y | V | T | K | H | V | K |
| D | R | F | L | S | S | Y | V | K |
| G | R | F | L | S | S | Y | V | K |
| D | S | F | L | S | S | Y | V | K |
| G | S | F | L | S | S | Y | V | K |
| D | R | Y | L | S | S | Y | V | K |
| G | R | Y | L | S | S | Y | V | K |
| D | S | Y | L | S | S | Y | V | K |
| G | S | Y | L | S | S | Y | V | K |
| D | R | F | V | S | S | Y | V | K |
| G | R | F | V | S | S | Y | V | K |
| D | S | F | V | S | S | Y | V | K |
| G | S | F | V | S | S | Y | V | K |
| D | R | Y | V | S | S | Y | V | K |
| G | R | Y | V | S | S | Y | V | K |
| D | S | Y | V | S | S | Y | V | K |
| G | S | Y | V | S | S | Y | V | K |
| D | R | F | L | T | S | Y | V | K |
| G | R | F | L | T | S | Y | V | K |
| D | S | F | L | T | S | Y | V | K |
| G | S | F | L | T | S | Y | V | K |
| D | R | Y | L | T | S | Y | V | K |
| G | R | Y | L | T | S | Y | V | K |
| D | S | Y | L | T | S | Y | V | K |
| G | S | Y | L | T | S | Y | V | K |
| D | R | F | V | T | S | Y | V | K |
| G | R | F | V | T | S | Y | V | K |
| D | S | F | V | T | S | Y | V | K |
| G | S | F | V | T | S | Y | V | K |
| D | R | Y | V | T | S | Y | V | K |
| G | R | Y | V | T | S | Y | V | K |
| D | S | Y | V | T | S | Y | V | K |
| G | S | Y | V | T | S | Y | V | K |
| D | R | F | L | S | K | Y | V | K |
| G | R | F | L | S | K | Y | V | K |
| D | S | F | L | S | K | Y | V | K |
| G | S | F | L | S | K | Y | V | K |
| D | R | Y | L | S | K | Y | V | K |
| G | R | Y | L | S | K | Y | V | K |
| D | S | Y | L | S | K | Y | V | K |
| G | S | Y | L | S | K | Y | V | K |
| D | R | F | V | S | K | Y | V | K |
| G | R | F | V | S | K | Y | V | K |
| D | S | F | V | S | K | Y | V | K |
| G | S | F | V | S | K | Y | V | K |
| D | R | Y | V | S | K | Y | V | K |
| G | R | Y | V | S | K | Y | V | K |
| D | S | Y | V | S | K | Y | V | K |
| G | S | Y | V | S | K | Y | V | K |
| D | R | F | L | T | K | Y | V | K |
| G | R | F | L | T | K | Y | V | K |
| D | S | F | L | T | K | Y | V | K |
| G | S | F | L | T | K | Y | V | K |
| D | R | Y | L | T | K | Y | V | K |
| G | R | Y | L | T | K | Y | V | K |
| D | S | Y | L | T | K | Y | V | K |
| G | S | Y | L | T | K | Y | V | K |
| D | R | F | V | T | K | Y | V | K |
| G | R | F | V | T | K | Y | V | K |
| D | S | F | V | T | K | Y | V | K |
| G | S | F | V | T | K | Y | V | K |
| D | R | Y | V | T | K | Y | V | K |
| G | R | Y | V | T | K | Y | V | K |
| D | S | Y | V | T | K | Y | V | K |
| G | S | Y | V | T | K | Y | V | K |
| D | R | F | L | S | S | H | A | K |
| G | R | F | L | S | S | H | A | K |
| D | S | F | L | S | S | H | A | K |
| G | S | F | L | S | S | H | A | K |
| D | R | Y | L | S | S | H | A | K |
| G | R | Y | L | S | S | H | A | K |
| D | S | Y | L | S | S | H | A | K |
| G | S | Y | L | S | S | H | A | K |
| D | R | F | V | S | S | H | A | K |
| G | R | F | V | S | S | H | A | K |
| D | S | F | V | S | S | H | A | K |
| G | S | F | V | S | S | H | A | K |
| D | R | Y | V | S | S | H | A | K |
| G | R | Y | V | S | S | H | A | K |
| D | S | Y | V | S | S | H | A | K |
| G | S | Y | V | S | S | H | A | K |
| D | R | F | L | T | S | H | A | K |
| G | R | F | L | T | S | H | A | K |
| D | S | F | L | T | S | H | A | K |
| G | S | F | L | T | S | H | A | K |
| D | R | Y | L | T | S | H | A | K |
| G | R | Y | L | T | S | H | A | K |
| D | S | Y | L | T | S | H | A | K |
| G | S | Y | L | T | S | H | A | K |
| D | R | F | V | T | S | H | A | K |
| G | R | F | V | T | S | H | A | K |
| D | S | F | V | T | S | H | A | K |
| G | S | F | V | T | S | H | A | K |
| D | R | Y | V | T | S | H | A | K |
| G | R | Y | V | T | S | H | A | K |
| D | S | Y | V | T | S | H | A | K |
| G | S | Y | V | T | S | H | A | K |
| D | R | F | L | S | K | H | A | K |
| G | R | F | L | S | K | H | A | K |
| D | S | F | L | S | K | H | A | K |
| G | S | F | L | S | K | H | A | K |
| D | R | Y | L | S | K | H | A | K |
| G | R | Y | L | S | K | H | A | K |
| D | S | Y | L | S | K | H | A | K |
| G | S | Y | L | S | K | H | A | K |
| D | R | F | V | S | K | H | A | K |
| G | R | F | V | S | K | H | A | K |
| D | S | F | V | S | K | H | A | K |
| G | S | F | V | S | K | H | A | K |
| D | R | Y | V | S | K | H | A | K |
| G | R | Y | V | S | K | H | A | K |
| D | S | Y | V | S | K | H | A | K |
| G | S | Y | V | S | K | H | A | K |
| D | R | F | L | T | K | H | A | K |
| G | R | F | L | T | K | H | A | K |
| D | S | F | L | T | K | H | A | K |
| G | S | F | L | T | K | H | A | K |
| D | R | Y | L | T | K | H | A | K |
| G | R | Y | L | T | K | H | A | K |
| D | S | Y | L | T | K | H | A | K |
| G | S | Y | L | T | K | H | A | K |
| D | R | F | V | T | K | H | A | K |
| G | R | F | V | T | K | H | A | K |
| D | S | F | V | T | K | H | A | K |
| G | S | F | V | T | K | H | A | K |
| D | R | Y | V | T | K | H | A | K |
| G | R | Y | V | T | K | H | A | K |
| D | S | Y | V | T | K | H | A | K |
| G | S | Y | V | T | K | H | A | K |
| D | R | F | L | S | S | Y | A | K |
| G | R | F | L | S | S | Y | A | K |
| D | S | F | L | S | S | Y | A | K |
| G | S | F | L | S | S | Y | A | K |
| D | R | Y | L | S | S | Y | A | K |
| G | R | Y | L | S | S | Y | A | K |
| D | S | Y | L | S | S | Y | A | K |
| G | S | Y | L | S | S | Y | A | K |
| D | R | F | V | S | S | Y | A | K |
| G | R | F | V | S | S | Y | A | K |
| D | S | F | V | S | S | Y | A | K |
| G | S | F | V | S | S | Y | A | K |
| D | R | Y | V | S | S | Y | A | K |
| G | R | Y | V | S | S | Y | A | K |
| D | S | Y | V | S | S | Y | A | K |
| G | S | Y | V | S | S | Y | A | K |
| D | R | F | L | T | S | Y | A | K |
| G | R | F | L | T | S | Y | A | K |
| D | S | F | L | T | S | Y | A | K |
| G | S | F | L | T | S | Y | A | K |
| D | R | Y | L | T | S | Y | A | K |
| G | R | Y | L | T | S | Y | A | K |
| D | S | Y | L | T | S | Y | A | K |
| G | S | Y | L | T | S | Y | A | K |
| D | R | F | V | T | S | Y | A | K |
| G | R | F | V | T | S | Y | A | K |
| D | S | F | V | T | S | Y | A | K |
| G | S | F | V | T | S | Y | A | K |
| D | R | Y | V | T | S | Y | A | K |
| G | R | Y | V | T | S | Y | A | K |
| D | S | Y | V | T | S | Y | A | K |
| G | S | Y | V | T | S | Y | A | K |
| D | R | F | L | S | K | Y | A | K |
| G | R | F | L | S | K | Y | A | K |
| D | S | F | L | S | K | Y | A | K |
| G | S | F | L | S | K | Y | A | K |
| D | R | Y | L | S | K | Y | A | K |
| G | R | Y | L | S | K | Y | A | K |
| D | S | Y | L | S | K | Y | A | K |
| G | S | Y | L | S | K | Y | A | K |
| D | R | F | V | S | K | Y | A | K |
| G | R | F | V | S | K | Y | A | K |
| D | S | F | V | S | K | Y | A | K |
| G | S | F | V | S | K | Y | A | K |
| D | R | Y | V | S | K | Y | A | K |
| G | R | Y | V | S | K | Y | A | K |
| D | S | Y | V | S | K | Y | A | K |
| G | S | Y | V | S | K | Y | A | K |
| D | R | F | L | T | K | Y | A | K |
| G | R | F | L | T | K | Y | A | K |
| D | S | F | L | T | K | Y | A | K |
| G | S | F | L | T | K | Y | A | K |
| D | R | Y | L | T | K | Y | A | K |
| G | R | Y | L | T | K | Y | A | K |
| D | S | Y | L | T | K | Y | A | K |
| G | S | Y | L | T | K | Y | A | K |
| D | R | F | V | T | K | Y | A | K |
| G | R | F | V | T | K | Y | A | K |
| D | S | F | V | T | K | Y | A | K |
| G | S | F | V | T | K | Y | A | K |
| D | R | Y | V | T | K | Y | A | K |
| G | R | Y | V | T | K | Y | A | K |
| D | S | Y | V | T | K | Y | A | K |
| G | S | Y | V | T | K | Y | A | K |

TABLE 9
Exemplary Mutations of mAB 15A1.2 Heavy Chain
(SEQ ID NO: 6) to Germline at the Indicated Residue Number

| 60 | 99 | 106 | 108 | 110 |
|---|---|---|---|---|
| F | G | F | W | W |
| Y | G | F | W | W |
| F | — | F | W | W |
| Y | — | F | W | W |
| F | G | L | W | W |
| Y | G | L | W | W |
| F | — | L | W | W |
| Y | — | L | W | W |
| F | G | F | — | W |
| Y | G | F | — | W |
| F | — | F | — | W |
| Y | — | F | — | W |
| F | G | L | — | W |
| Y | G | L | — | W |
| F | — | L | — | W |
| Y | — | L | — | W |
| F | G | F | W | M |
| Y | G | F | W | M |
| F | — | F | W | M |
| Y | — | F | W | M |
| F | G | L | W | M |
| Y | G | L | W | M |
| F | — | L | W | M |
| Y | — | L | W | M |
| F | G | F | — | M |
| Y | G | F | — | M |
| F | — | F | — | M |
| Y | — | F | — | M |
| F | G | L | — | M |
| Y | G | L | — | M |
| F | — | L | — | M |
| Y | — | L | — | M |

“—” indicates the absence of a residue at that position with reference to SEQ ID NO: 6

TABLE 10
Exemplary Mutations of mAB 15A1.2 Light Chain (SEQ ID NO: 8) to
Germline at the Indicated Residue Number

| 31 | 39 | 45 | 50 | 92 | 96 |
|---|---|---|---|---|---|
| N | T | Q | G | D | P |
| S | T | Q | G | D | P |
| N | K | Q | G | D | P |
| S | K | Q | G | D | P |
| N | T | K | G | D | P |
| S | T | K | G | D | P |
| N | K | K | G | D | P |
| S | K | K | G | D | P |
| N | T | Q | A | D | P |
| S | T | Q | A | D | P |
| N | K | Q | A | D | P |
| S | K | Q | A | D | P |
| N | T | K | A | D | P |
| S | T | K | A | D | P |
| N | K | K | A | D | P |
| S | K | K | A | D | P |
| N | T | Q | G | N | P |
| S | T | Q | G | N | P |
| N | K | Q | G | N | P |
| S | K | Q | G | N | P |
| N | T | K | G | N | P |
| S | T | K | G | N | P |
| N | K | K | G | N | P |
| S | K | K | G | N | P |
| N | T | Q | A | N | P |
| S | T | Q | A | N | P |
| N | K | Q | A | N | P |
| S | K | Q | A | N | P |
| N | T | K | A | N | P |
| S | T | K | A | N | P |
| N | K | K | A | N | P |
| S | K | K | A | N | P |
| N | T | Q | G | D | W |
| S | T | Q | G | D | W |
| N | K | Q | G | D | W |
| S | K | Q | G | D | W |
| N | T | K | G | D | W |
| S | T | K | G | D | W |
| N | K | K | G | D | W |
| S | K | K | G | D | W |
| N | T | Q | A | D | W |
| S | T | Q | A | D | W |
| N | K | Q | A | D | W |
| S | K | Q | A | D | W |
| N | T | K | A | D | W |
| S | T | K | A | D | W |
| N | K | K | A | D | W |
| S | K | K | A | D | W |
| N | T | Q | G | N | W |
| S | T | Q | G | N | W |
| N | K | Q | G | N | W |
| S | K | Q | G | N | W |
| N | T | K | G | N | W |
| S | T | K | G | N | W |
| N | K | K | G | N | W |
| S | K | K | G | N | W |
| N | T | Q | A | N | W |
| S | T | Q | A | N | W |
| N | K | Q | A | N | W |
| S | K | Q | A | N | W |
| N | T | K | A | N | W |
| S | T | K | A | N | W |
| N | K | K | A | N | W |
| S | K | K | A | N | W |

TABLE 11
Exemplary Mutations of mAB 15A11.1 Heavy Chain (SEQ ID NO: 10) to
Germline at the Indicated Residue Number

| 27 | 40 | 72 | 99 | 100 | 105 | 106 | 107 |
|---|---|---|---|---|---|---|---|
| D | T | G | E | G | S | E | E |
| Y | T | G | E | G | S | E | E |
| D | A | G | E | G | S | E | E |
| Y | A | G | E | G | S | E | E |
| D | T | E | E | G | S | E | E |
| Y | T | E | E | G | S | E | E |
| D | A | E | E | G | S | E | E |
| Y | A | E | E | G | S | E | E |
| D | T | G | — | G | S | E | E |
| Y | T | G | — | G | S | E | E |
| D | A | G | — | G | S | E | E |
| Y | A | G | — | G | S | E | E |
| D | T | E | — | G | S | E | E |
| Y | T | E | — | G | S | E | E |
| D | A | E | — | G | S | E | E |
| Y | A | E | — | G | S | E | E |
| D | T | G | E | — | S | E | E |
| Y | T | G | E | — | S | E | E |
| D | A | G | E | — | S | E | E |
| Y | A | G | E | — | S | E | E |
| D | T | E | E | — | S | E | E |
| Y | T | E | E | — | S | E | E |
| D | A | E | E | — | S | E | E |
| Y | A | E | E | — | S | E | E |
| D | T | G | — | — | S | E | E |
| Y | T | G | — | — | S | E | E |
| D | A | G | — | — | S | E | E |
| Y | A | G | — | — | S | E | E |
| D | T | E | — | — | S | E | E |
| Y | T | E | — | — | S | E | E |
| D | A | E | — | — | S | E | E |
| Y | A | E | — | — | S | E | E |
| D | T | G | E | G | — | E | E |
| Y | T | G | E | G | — | E | E |
| D | A | G | E | G | — | E | E |
| Y | A | G | E | G | — | E | E |
| D | T | E | E | G | — | E | E |
| Y | T | E | E | G | — | E | E |
| D | A | E | E | G | — | E | E |
| Y | A | E | E | G | — | E | E |
| D | T | G | — | G | — | E | E |
| Y | T | G | — | G | — | E | E |
| D | A | G | — | G | — | E | E |
| Y | A | G | — | G | — | E | E |
| D | T | E | — | G | — | E | E |
| Y | T | E | — | G | — | E | E |
| D | A | E | — | G | — | E | E |
| Y | A | E | — | G | — | E | E |
| D | T | G | E | — | — | E | E |
| Y | T | G | E | — | — | E | E |
| D | A | G | E | — | — | E | E |
| Y | A | G | E | — | — | E | E |
| D | T | E | E | — | — | E | E |
| Y | T | E | E | — | — | E | E |
| D | A | E | E | — | — | E | E |
| Y | A | E | E | — | — | E | E |
| D | T | G | — | — | — | E | E |
| Y | T | G | — | — | — | E | E |
| D | A | G | — | — | — | E | E |
| Y | A | G | — | — | — | E | E |
| D | T | E | — | — | — | E | E |
| Y | T | E | — | — | — | E | E |
| D | A | E | — | — | — | E | E |
| Y | A | E | — | — | — | E | E |
| D | T | G | E | G | S | — | E |
| Y | T | G | E | G | S | — | E |
| D | A | G | E | G | S | — | E |
| Y | A | G | E | G | S | — | E |
| D | T | E | E | G | S | — | E |
| Y | T | E | E | G | S | — | E |
| D | A | E | E | G | S | — | E |
| Y | A | E | E | G | S | — | E |
| D | T | G | — | G | S | — | E |
| Y | T | G | — | G | S | — | E |
| D | A | G | — | G | S | — | E |
| Y | A | G | — | G | S | — | E |
| D | T | E | — | G | S | — | E |
| Y | T | E | — | G | S | — | E |
| D | A | E | — | G | S | — | E |
| Y | A | E | — | G | S | — | E |
| D | T | G | E | — | S | — | E |
| Y | T | G | E | — | S | — | E |
| D | A | G | E | — | S | — | E |
| Y | A | G | E | — | S | — | E |
| D | T | E | E | — | S | — | E |
| Y | T | E | E | — | S | — | E |
| D | A | E | E | — | S | — | E |
| Y | A | E | E | — | S | — | E |
| D | T | G | — | — | S | — | E |
| Y | T | G | — | — | S | — | E |
| D | A | G | — | — | S | — | E |
| Y | A | G | — | — | S | — | E |
| D | T | E | — | — | S | — | E |
| Y | T | E | — | — | S | — | E |
| D | A | E | — | — | S | — | E |
| Y | A | E | — | — | S | — | E |
| D | T | G | E | G | — | — | E |
| Y | T | G | E | G | — | — | E |
| D | A | G | E | G | — | — | E |
| Y | A | G | E | G | — | — | E |
| D | T | E | E | G | — | — | E |
| Y | T | E | E | G | — | — | E |
| D | A | E | E | G | — | — | E |
| Y | A | E | E | G | — | — | E |
| D | T | G | — | G | — | — | E |
| Y | T | G | — | G | — | — | E |
| D | A | G | — | G | — | — | E |
| Y | A | G | — | G | — | — | E |
| D | T | E | — | G | — | — | E |
| Y | T | E | — | G | — | — | E |
| D | A | E | — | G | — | — | E |
| Y | A | E | — | G | — | — | E |
| D | T | G | E | — | — | — | E |
| Y | T | G | E | — | — | — | E |
| D | A | G | E | — | — | — | E |
| Y | A | G | E | — | — | — | E |
| D | T | E | E | — | — | — | E |
| Y | T | E | E | — | — | — | E |
| D | A | E | E | — | — | — | E |
| Y | A | E | E | — | — | — | E |
| D | T | G | — | — | — | — | E |
| Y | T | G | — | — | — | — | E |
| D | A | G | — | — | — | — | E |
| Y | A | G | — | — | — | — | E |
| D | T | E | — | — | — | — | E |
| Y | T | E | — | — | — | — | E |
| D | A | E | — | — | — | — | E |
| Y | A | E | — | — | — | — | E |
| D | T | G | E | G | S | E | — |
| Y | T | G | E | G | S | E | — |
| D | A | G | E | G | S | E | — |
| Y | A | G | E | G | S | E | — |
| D | T | E | E | G | S | E | — |
| Y | T | E | E | G | S | E | — |
| D | A | E | E | G | S | E | — |
| Y | A | E | E | G | S | E | — |
| D | T | G | — | G | S | E | — |
| Y | T | G | — | G | S | E | — |
| D | A | G | — | G | S | E | — |
| Y | A | G | — | G | S | E | — |
| D | T | E | — | G | S | E | — |
| Y | T | E | — | G | S | E | — |
| D | A | E | — | G | S | E | — |
| Y | A | E | — | G | S | E | — |
| D | T | G | E | — | S | E | — |
| Y | T | G | E | — | S | E | — |
| D | A | G | E | — | S | E | — |
| Y | A | G | E | — | S | E | — |
| D | T | E | E | — | S | E | — |
| Y | T | E | E | — | S | E | — |
| D | A | E | E | — | S | E | — |
| Y | A | E | E | — | S | E | — |
| D | T | G | — | — | S | E | — |
| Y | T | G | — | — | S | E | — |
| D | A | G | — | — | S | E | — |
| Y | A | G | — | — | S | E | — |
| D | T | E | — | — | S | E | — |
| Y | T | E | — | — | S | E | — |
| D | A | E | — | — | S | E | — |
| Y | A | E | — | — | S | E | — |
| D | T | G | E | G | — | E | — |
| Y | T | G | E | G | — | E | — |
| D | A | G | E | G | — | E | — |
| Y | A | G | E | G | — | E | — |
| D | T | E | E | G | — | E | — |
| Y | T | E | E | G | — | E | — |
| D | A | E | E | G | — | E | — |
| Y | A | E | E | G | — | E | — |
| D | T | G | — | G | — | E | — |
| Y | T | G | — | G | — | E | — |
| D | A | G | — | G | — | E | — |
| Y | A | G | — | G | — | E | — |
| D | T | E | — | G | — | E | — |
| Y | T | E | — | G | — | E | — |
| D | A | E | — | G | — | E | — |
| Y | A | E | — | G | — | E | — |
| D | T | G | E | — | — | E | — |
| Y | T | G | E | — | — | E | — |
| D | A | G | E | — | — | E | — |
| Y | A | G | E | — | — | E | — |
| D | T | E | E | — | — | E | — |
| Y | T | E | E | — | — | E | — |
| D | A | E | E | — | — | E | — |
| Y | A | E | E | — | — | E | — |
| D | T | G | — | — | — | E | — |
| Y | T | G | — | — | — | E | — |
| D | A | G | — | — | — | E | — |
| Y | A | G | — | — | — | E | — |
| D | T | E | — | — | — | E | — |
| Y | T | E | — | — | — | E | — |
| D | A | E | — | — | — | E | — |
| Y | A | E | — | — | — | E | — |
| D | T | G | E | G | S | — | — |
| Y | T | G | E | G | S | — | — |
| D | A | G | E | G | S | — | — |
| Y | A | G | E | G | S | — | — |
| D | T | E | E | G | S | — | — |
| Y | T | E | E | G | S | — | — |
| D | A | E | E | G | S | — | — |
| Y | A | E | E | G | S | — | — |
| D | T | G | — | G | S | — | — |
| Y | T | G | — | G | S | — | — |
| D | A | G | — | G | S | — | — |
| Y | A | G | — | G | S | — | — |
| D | T | E | — | G | S | — | — |
| Y | T | E | — | G | S | — | — |
| D | A | E | — | G | S | — | — |
| Y | A | E | — | G | S | — | — |
| D | T | G | E | — | S | — | — |
| Y | T | G | E | — | S | — | — |
| D | A | G | E | — | S | — | — |
| Y | A | G | E | — | S | — | — |
| D | T | E | E | — | S | — | — |
| Y | T | E | E | — | S | — | — |
| D | A | E | E | — | S | — | — |
| Y | A | E | E | — | S | — | — |
| D | T | G | — | — | S | — | — |
| Y | T | G | — | — | S | — | — |
| D | A | G | — | — | S | — | — |
| Y | A | G | — | — | S | — | — |
| D | T | E | — | — | S | — | — |
| Y | T | E | — | — | S | — | — |
| D | A | E | — | — | S | — | — |
| Y | A | E | — | — | S | — | — |
| D | T | G | E | G | — | — | — |
| Y | T | G | E | G | — | — | — |
| D | A | G | E | G | — | — | — |
| Y | A | G | E | G | — | — | — |
| D | T | E | E | G | — | — | — |
| Y | T | E | E | G | — | — | — |
| D | A | E | E | G | — | — | — |
| Y | A | E | E | G | — | — | — |
| D | T | G | — | G | — | — | — |
| Y | T | G | — | G | — | — | — |
| D | A | G | — | G | — | — | — |
| Y | A | G | — | G | — | — | — |
| D | T | E | — | G | — | — | — |
| Y | T | E | — | G | — | — | — |
| D | A | E | — | G | — | — | — |
| Y | A | E | — | G | — | — | — |
| D | T | G | E | — | — | — | — |
| Y | T | G | E | — | — | — | — |
| D | A | G | E | — | — | — | — |
| Y | A | G | E | — | — | — | — |
| D | T | E | E | — | — | — | — |
| Y | T | E | E | — | — | — | — |
| D | A | E | E | — | — | — | — |
| Y | A | E | E | — | — | — | — |
| D | T | G | — | — | — | — | — |
| Y | T | G | — | — | — | — | — |
| D | A | G | — | — | — | — | — |
| Y | A | G | — | — | — | — | — |
| D | T | E | — | — | — | — | — |
| Y | T | E | — | — | — | — | — |
| D | A | E | — | — | — | — | — |
| Y | A | E | — | — | — | — | — |

“—” indicates the absence of a residue at that position with reference to SEQ ID NO: 10

TABLE 12
Exemplary Mutations of mAB 15A11.1 Light Chain
(SEQ ID NO: 12) to Germline at the Indicated Residue Number
93
R
S

### Example 8

### Expression of Human Heparanase in Human Tumour Cells

MCF7 and MDA MB231 breast tumour cells that expressed little endogenous heparanase were transfected with an expression construct comprising a full-length human heparanase cDNA under the control of a CMV promoter in a pCR3.1 Bid vector. Selection and subcloning of transfected cells led to the isolation of cell lines expressing heparanase, MCF7-Hpa1#19 and MDA MB231-Hpa1#4.

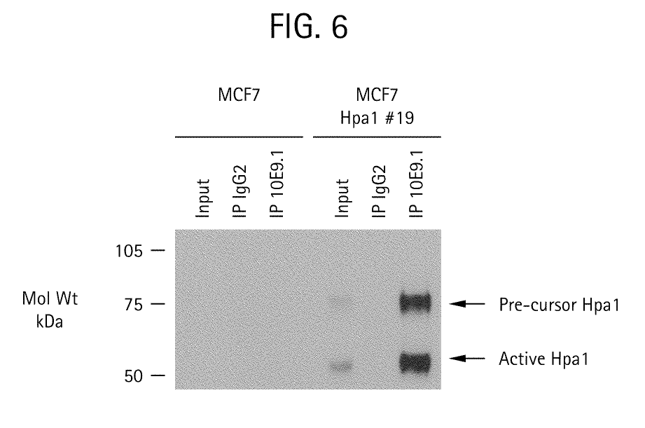

Heparanase expression and specificity of anti-heparanase antibodies was demonstrated by immunoprecipitation and Western blotting. Briefly, protein lysates (400 μg in RIPA lysis buffer—137 mM NaCl, 20 mM Tris pH7.4, 10% glycerol, 1% Triton, 0.5% Sodium deoxycholate, 0.1% SDS, 2 mM EDTA, 1 mM dithiothreitol, 4 mM pefabloc, 10 mM glycerol-2-phosphate, 5 mM NaF and protease inhibitor cocktail) from both parental and heparanase transfected cells were incubated with 10 μg IgG2 control or anti-heparanase (10E9.1) antibodies for 1 hour at 4° C. Following immunoprecipitation with protein G agarose beads and washing with RIPA buffer, samples were separated on 10% bis-tris gels and transferred to nitrocellulose for Western blotting. Heparanase was detected using an anti-heparanase polyclonal antibody (Biochain Cat. No. Z5010104), that recognises both precursor and active heparanase. Both MCF7 and MDA MB231 heparanase transfected cells were shown to express significant levels of both precursor (65 kDa) and active heparanase (50 kDa). The specificity of anti-human heparanase antibody 10E9.1 was shown by immunoprecipitation of both precursor and active forms of heparanase; representative data from MCF7 parental and MCF7-Hpa1#19 cells are shown in FIG. 6.

### Example 9

Treatment of Cells with Anti-Heparanase Antibodies

MDA MB231-Hpa1#4 cells were cultured in the presence of 25 μg per ml anti-human heparanase antibody or control IgG2 antibody (Sigma Cat. No. 1504-1MG) for 72 hrs. Following the removal of culture medium, protein extracts were prepared from the cells in 100 μl RIPA buffer and separated on agarose gels and transferred to nitrocellulose as described above. Western blots were probed with an anti-heparanase antibody that detected only pro-heparanase (SantaCruz Cat. No. SC25825). Equal protein loading of the lanes was demonstrated by Western blotting for GAPDH (glyceraldehyde phosphate dehydrogenase) using mouse monoconal 6C5 (Ambion/In Vitrogen Cat. No. AM4300).

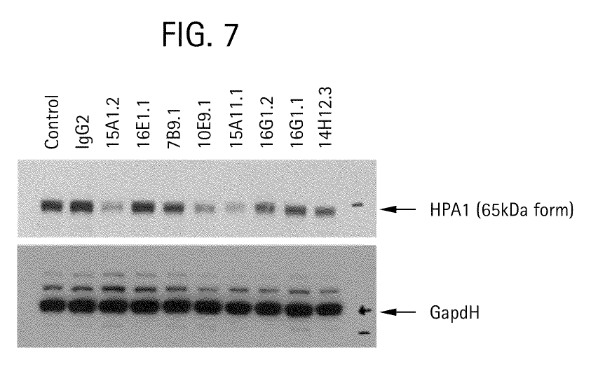

Treatment with 10E9.1, 15A1.2 and 15A11.1 resulted in a significant inhibition of heparanase expression compared to untreated or IgG2 treated control (FIG. 7). Antibodies that were less effective inhibitors of heparanase, or less effective binders to immobilised heparanase, were less effective inhibitors of heparanase expression.

MCF7-Hpa1#19 cells were cultured until confluent and then grown overnight (˜12 hrs) at 37° C. in serum free medium. Following overnight incubation, culture medium was replaced with serum free medium containing 25 μg per ml anti-heparanase monoclonal antibody 10E9.1 or a control IgG2 antibody (Sigma Cat. No. 1504-1MG) and incubated for a further 24 hrs at 37° C. Cell cultures were then treated for 2 hrs with PMA 0.1-10 μM, to stimulate heparanase expression (Sasaki N et al, J Immunol. 172: 3830, 2004). Conditioned medium from the cultures was harvested and concentrated 10-fold and protein lysates in RIPA buffer were prepared from the cells. Proteins from conditioned medium or cell lysates were separated on agarose gels as described previously and heparanase was detected using an anti-heparanase polyclonal antibody (Biochain Cat. No. Z5010104).

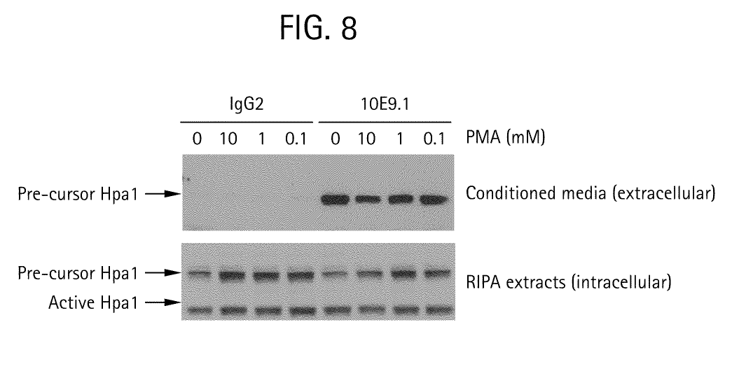

Treatment of cells with mAb 10E9.1 resulted in an accumulation of precursor (65 kDa) heparanase in the culture medium compared to cells treated with the control IgG2 antibody (FIG. 8). It was noteworthy that no active heparanase (50 kDa) was detected in the conditioned medium, whereas the cell lysates from both IgG2 and 10E9.1 treated cells clearly showed the presence of intracellular precursor and active heparanase. Stimulation of MCF7-Hpa1#19 cells with PMA resulted in an increase in intracellular heparanase, which appeared to be attenuated in cells treated with 10E9.1.

Reduced expression of heparanase and accumulation of the precursor form of heparanase following treatment with the anti-human heparanase antibody suggests that in addition to inhibiting enzyme activity, the anti-heparanase antibodies are inhibiting expression and processing of heparanase. Although additional experiments would be required to determine the precise mechanism, binding of anti-heparanase antibodies to the precursor protein may have inhibited efficient uptake of the protein into the cell, subsequent processing and secretion of the active form of heparanase. The fact that total levels of heparanase are also reduced by treatment with the anti-heparanase antibodies (FIG. 7) suggests that there may be a feedback mechanism leading to reduced levels of intracellular heparanase (see FIG. 8).

The data suggest that in addition to inhibiting heparanase enzyme activity, the anti-human heparanase antibodies may also reduce the overall expression of heparanase in targeted tissues and cells.

### Example 10

### Binding of Anti-Heparanase Antibodies to Tumour Cells

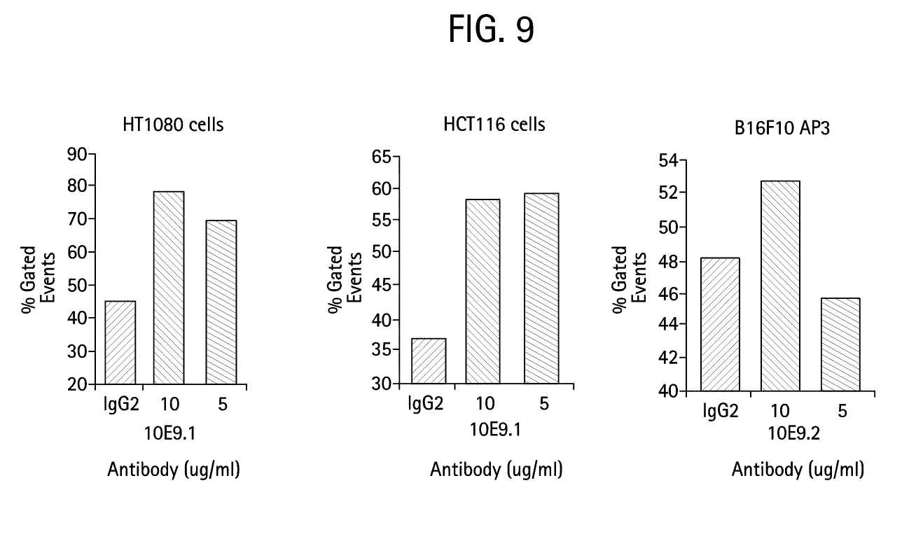

Although heparanase is found as a secreted protein, a proportion of heparanase is found in conjunction with cell surface heparan sulfate. The ability of anti-human heparanase antibodies to bind to cell surface heparanase was tested in a FACS assay. HT1080 human fibrosarcoma cells, HCT116 human colorectal tumour cells and B16F10 AP3 murine melanoma cells were each seeded into 6-well plates and cultured for 48 hrs prior to the addition of anti-heparanase antibody 10E9.1 or control IgG2 antibody (Sigma Cat. No. 1504-1MG). Antibodies were added to the cells to a final concentration of 5 or 10 μg per ml and incubated with the cells for 2 hrs at 37° C. The cells were then washed with phosphate buffered saline (PBS) and harvested by incubation with trypsin. The cells were pelleted by centrifugation and resuspended in 0.5 ml PBS, 0.1% BSA containing 1/200 dilution of AlexaFluor 488 anti-human IgG. The cells were incubated for 1 hr at room temperature, washed with PBS/BSA, resuspended in PBS and analysed by FACS. Anti-heparanase 10E9.1 was shown to bind extensively to HT1080 and HCT116 cells (FIGS. 9a and 9b). In agreement with the cross species binding and inhibition of murine heparanase data presented hereinabove, 10E9.1 was also shown to bind to the cell surface of B16F10 AP3 murine melanoma cells (FIG. 9c). In the case of the murine tumour cells, the increase of specific binding compared to the IgG2 control antibody was less than that observed for the human tumour cells, and the signal observed at 5 μg per ml 10E9.1 was significantly less than that of the control antibody. The differences likely reflect a lower affinity for murine heparanase in addition to any differences in absolute heparanase expression.

### Example 11

### Anti-Heparanase Antibodies Inhibit Cell Surface Heparanase Activity

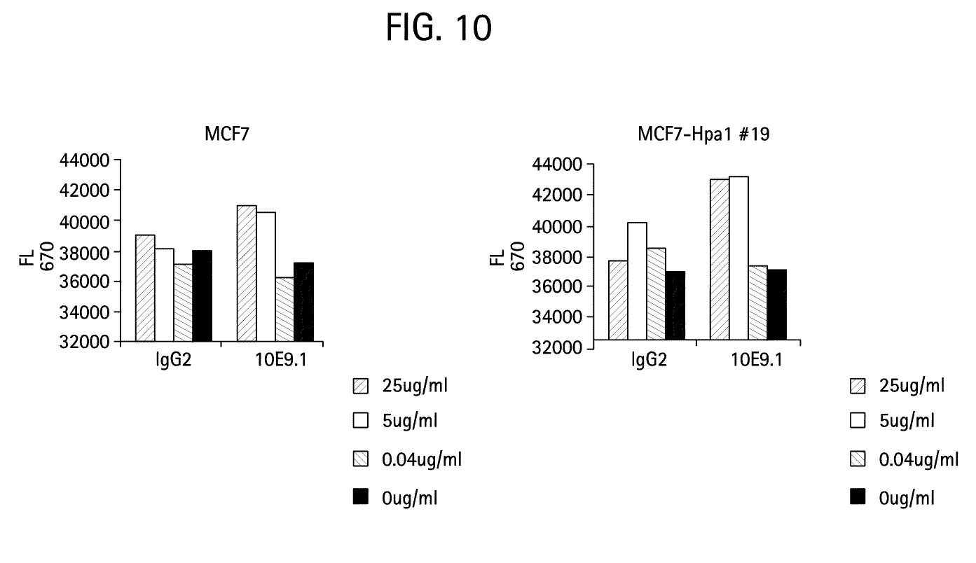

Cell surface heparanase activity was measured by a modification of the methodology used in the recombinant enzyme screen (Example 4). Parental MCF7 or MCF7-Hpa1#19 cells were cultured in non-tissue culture (non-adherent) 96-well plates, seeded at 10⁶ cells per well. After overnight incubation at 37° C. anti-heparanase 10E9.1 or control IgG2 (Sigma Cat. 1504-1MG) antibodies were added to the cells, to a final concentration of 0.04-25 μg per ml, and incubated for 1 hr at room temperature. Thereafter Biotin Heparan Sulfate Europium Criptate (CisBio Cat. No. 61BHSKAA) was added to the cells (final concentration 20 ng per ml) and incubated at 37° C. for 1 hr. Degradation of the heparan sulfate substrate was determined following the addition of Streptavidin-XL665 (final concentration 250 ng per ml; CisBio Cat. No. 611SAXLA) to the cells and fluorescence measured using excitation at 330 nm and emission at 670 nm. In the absence of anti-heparanase antibody or in the presence of control IgG2, the minimum fluorescence at 670 nm is the result of degradation of heparan sulfate (see FIG. 10). In contrast, incubation with 10E9.1 at a concentration of 5 or 25 μg per ml resulted in complete inhibition of cell surface heparanase activity (high fluorescence at 670 nm). As the fluorescence values at 5 and 25 μg per ml of 10E9.1 are very similar, this likely represents total inhibition of available cell surface heparanase activity by 10E9.1.

### Example 12

### In Vivo Activity of Anti-Heparanase Antibodies

MCF7-Hpa1#19 and HCT116 tumour cells were harvested from sub-confluent cultures. A total of 5×10⁶ (MCF7-Hpa1#19) or 10⁷ (HCT116) tumour cells were inoculated subcutaneously into the flank of male SCID mice. HCT116 tumour cells were implanted in PBS; MCF7-HPA1#9 cells were implanted in PBS containing 50% matrigel.

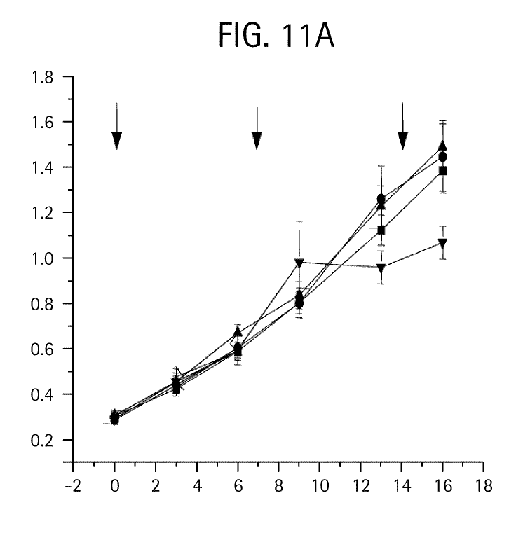

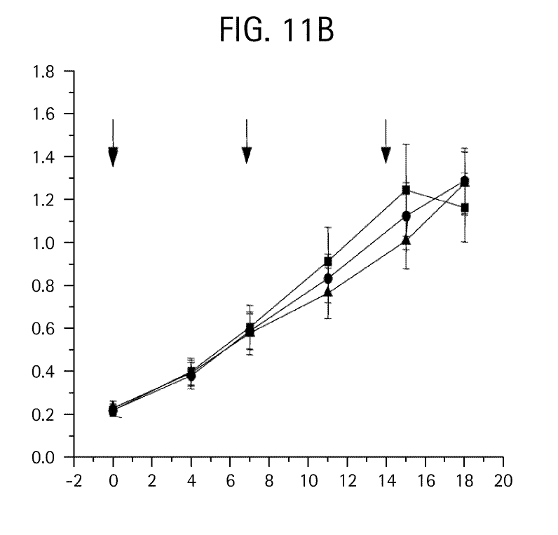

When tumours had attained ˜200 mm³, tumour bearing mice were randomised into treatment groups and dosed with the following; PBS; an isotype matched control IgG2 antibody, 15 mg per kg; anti-heparanase antibody 10E9.1, 7.5 or 15 mg per kg. All treatments were administered once weekly i.p. Tumour growth was measured twice per week using calipers and tumour volumes calculated according to an ellipsoid volume is formula (3.142/6×length×width×width) from measures of the largest tumour diameter and two opposing widths (FIG. 11).

MCF7-Hpa1#19 or HCT116 tumour growth was not inhibited by 10E9.1 and similar data was obtained with anti-heparanase 15A1.2 and 15A11.1 antibodies (data not shown). Both tumour cell lines were known to express active heparanase. The lack of inhibition of tumor growth exhibited by the antibodies is likely a reflection that this subcutaneous tumour growth model is inappropriate for testing the in vivo activity of the antibodies.

Heparanase activity is thought to contribute to tumour progression by requiring contributions from both host and tumour heparan sulfate compartments which may not be correctly represented in subcutaneous model above. It is believed that metastatic tumor models such as those described below and in Yang et al, Blood 105:1303, 2005; Sanderson et al, J Cellular Biochem 96: 897, 2005 are likely to be more appropriate model for assessing the efficacy of the anti-heparanase antibodies.

### Example 13

### In Vivo Activity of Anti-Heparanase Antibodies

Treatment with anti-human heparanase antibodies to test the effect of heparanase inhibition on the contribution of heparanase and modification/degradation of heparan sulfate to tumour invasion and tumour growth is tested in the context of the tumour microenvironment.

MDA MB231 breast tumour cells are implanted in the mammary fat pad and grow as a primary tumour and form distant metastases in the lungs after approximately 4 weeks. After tumour cell transplantation and when palpable tumours (˜100 mm³) have formed, mice are randomised into groups and treated with anti-human heparanase or control antibodies. Tumour bearing mice are randomised into treatment groups and dosed with the following; PBS; an isotype matched control IgG2 antibody, 15 mg per kg; anti-heparanase antibody 10E9.1, 15A11.1 or 15A1.2, 7.5 or 15 mg per kg. All treatments are administered once weekly i.p. Tumour growth in the mammary fat pad is measured twice per week using calipers and tumour volumes calculated according to an ellipsoid volume formula (3.142/6×length×width×width) from measures of the largest tumour diameter and two opposing widths. Lung tumour burden is measured by direct counting of lung metastases, weight of lungs, or by histological examination, compared to equivalent measures from control animals.

Alternatively, injection of tumour cells into the tail vein frequently gives rise to colonies of tumour cells growing in the lungs; or tumour cells may be injected into the spleen whereby tumour colonies develop in the liver; or tumour cells may be directly injected into the tibia whereby tumours develop in the bone. In each of these models, treatment with anti-human heparanase antibodies would test the effect of heparanase inhibition on tumour growth and invasion in an organ-specific heparan sulfate microenvironment.

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An antibody that specifically binds to heparanase and inhibits heparanase enzyme activity, wherein the antibody also binds to and inhibits murine and cynomolgus heparanase enzyme activity.

2. An antibody that specifically binds to heparanase with a Kd of less than 200 pM and inhibits heparanase enzyme activity.

3. The antibody of claim 1 that specifically binds to pro-heparanase and thereby inhibits the activation of pro-heparanase to heparanase.

4-5. (canceled)

6. An antibody according to claim 1, wherein the antibody comprises any one or more of:
(a)(i) a VH CDR1 having an amino acid sequence identical to or comprising 1, 2, or 3 amino acid residue substitutions relative to the Kabat VH CDR1 sequence of monoclonal antibody 10E9.1 (SEQ ID NO.:47);
(ii) a VH CDR2 having an amino acid sequence identical to or comprising 1, 2, or 3 amino acid residue substitutions relative to the Kabat VH CDR2 sequence of monoclonal antibody 10E9.1 (SEQ ID NO.:48);
(iii) a VH CDR3 having an amino acid sequence identical to or comprising 1, 2, or 3 amino acid residue substitutions relative to the Kabat VH CDR3 sequence of monoclonal antibody 10E9.1 (SEQ ID NO.:49);
(iv) a VL CDR1 having an amino acid sequence identical to or comprising 1, 2, or 3 amino acid residue substitutions relative to the Kabat VL CDR1 sequence of monoclonal antibody 10E9.1 (SEQ ID NO.:56);
(v) a VL CDR2 having an amino acid sequence identical to or comprising 1, 2, or 3 amino acid residue substitutions relative to the Kabat VL CDR2 sequence of monoclonal antibody 10E9.1 (SEQ ID NO.:57); and
(vi) a VL CDR3 having an amino acid sequence identical to or comprising 1, 2, or 3 amino acid residue substitutions relative to the Kabat VL CDR3 sequence of monoclonal antibody 10E9.1 (SEQ ID NO.:58), or
(b) (i) a VH CDR1 having an amino acid sequence identical to or comprising 1, 2, or 3 amino acid residue substitutions relative to the Kabat VH CDR1 sequence of monoclonal antibody 15A1.2 (SEQ ID NO.:53);
(ii) a VH CDR2 having an amino acid sequence identical to or comprising 1, 2, or 3 amino acid residue substitutions relative to the Kabat VH CDR2 sequence of monoclonal antibody 15A1.2 (SEQ ID NO.:54);
(iii) a VH CDR3 having an amino acid sequence identical to or comprising 1, 2, or 3 amino acid residue substitutions relative to the Kabat VH CDR3 sequence of monoclonal antibody 15A1.2 (SEQ ID NO.:55);
(iv) a VL CDR1 having an amino acid sequence identical to or comprising 1, 2, or 3 amino acid residue substitutions relative to the Kabat VL CDR1 sequence of monoclonal antibody 15A1.2 (SEQ ID NO.:62);
(v) a VL CDR2 having an amino acid sequence identical to or comprising 1, 2, or 3 amino acid residue substitutions relative to the Kabat VL CDR2 sequence of monoclonal antibody 15A1.2 (SEQ ID NO.:63); and
(vi) a VL CDR3 having an amino acid sequence identical to or comprising 1, 2, or 3 amino acid residue substitutions relative to the Kabat VL CDR3 sequence of monoclonal antibody 15A1.2 (SEQ ID NO.:64), or
(c) (i) a VH CDR1 having an amino acid sequence identical to or comprising 1, 2, or 3 amino acid residue substitutions relative to the Kabat VH CDR1 sequence of monoclonal antibody 15A11.1 (SEQ ID NO.:50);
(ii) a VH CDR2 having an amino acid sequence identical to or comprising 1, 2, or 3 amino acid residue substitutions relative to the Kabat VH CDR2 sequence of monoclonal antibody 15A11.1 (SEQ ID NO.:51);
(iii) a VH CDR3 having an amino acid sequence identical to or comprising 1, 2, or 3 amino acid residue substitutions relative to the Kabat VH CDR3 sequence of monoclonal antibody 15A11.1 (SEQ ID NO.:52);
(iv) a VL CDR1 having an amino acid sequence identical to or comprising 1, 2, or 3 amino acid residue substitutions relative to the Kabat VL CDR1 sequence of monoclonal antibody 15A11.1 (SEQ ID NO.:59);
(v) a VL CDR2 having an amino acid sequence identical to or comprising 1, 2, or 3 amino acid residue substitutions relative to the Kabat VL CDR2 sequence of monoclonal antibody 15A11.1 (SEQ ID NO.:60); and
(vi) a VL CDR3 having an amino acid sequence identical to or comprising 1, 2, or 3 amino acid residue substitutions relative to the Kabat VL CDR3 sequence of monoclonal antibody 15A11.1 (SEQ ID NO.:61).

7. An antibody according to claim 6, wherein the antibody comprises any one or more of:
(a) (i) a VH CDR1 having the amino acid sequence of VH CDR1 of monoclonal antibody 10E9.1 (SEQ ID NO.:47);
(ii) a VH CDR2 having the amino acid sequence of VH CDR2 of monoclonal antibody 10E9.1 (SEQ ID NO.:48);
(iii) a VH CDR3 having the amino acid sequence of VH CDR3 of monoclonal antibody 10E9.1 (SEQ ID NO.:49);
(iv) a VL CDR1 having the amino acid sequence of VL CDR1 of monoclonal antibody 10E9.1 (SEQ ID NO.:56);
(v) a VL CDR2 having the amino acid sequence of VL CDR2 of monoclonal antibody 10E9.1 (SEQ ID NO.:57); and
(vi) a VL CDR3 having the amino acid sequence of VL CDR3 of monoclonal antibody 10E9.1 (SEQ ID NO.:58), or
(b) (i) a VH CDR1 having the amino acid sequence of the VH CDR1 of monoclonal antibody 15A1.2 (SEQ ID NO.:53);
(ii) a VH CDR2 having the amino acid sequence of the VH CDR2 of monoclonal antibody 15A1.2 (SEQ ID NO.:54);
(iii) a VH CDR3 having the amino acid sequence of the VH CDR3 of monoclonal antibody 15A1.2 (SEQ ID NO.:55);
(iv) a VL CDR1 having the amino acid sequence of the VL CDR1 of monoclonal antibody 15A1.2 (SEQ ID NO.:62);
(v) a VL CDR2 having the amino acid sequence of the VL CDR2 of monoclonal antibody 15A1.2 (SEQ ID NO.:63); and
(vi) a VL CDR3 having the amino acid sequence of the VL CDR3 of monoclonal antibody 15A1.2 (SEQ ID NO.:64), or
(c) (i) a VH CDR1 having the amino acid sequence of the VH CDR1 of monoclonal antibody 15A11.1 (SEQ ID NO.:50);
(ii) a VH CDR2 having the amino acid sequence of the VH CDR2 of monoclonal antibody 15A11.1 (SEQ ID NO.:51);
(iii) a VH CDR3 having the amino acid sequence of the VH CDR3 of monoclonal antibody 15A11.1 (SEQ ID NO.:52);
(iv) a VL CDR1 having the amino acid sequence of the VL CDR1 of monoclonal antibody 15A11.1 (SEQ ID NO.:59);
(v) a VL CDR2 having the amino acid sequence of the VL CDR2 of monoclonal antibody 15A11.1 (SEQ ID NO.:60); and
(vi) a VL CDR3 having the amino acid sequence of the VL CDR3 of monoclonal antibody 15A11.1 (SEQ ID NO.:61).

8. An antibody according to claim 6, wherein the antibody comprises a VH chain domain comprising three CDRs and a VL chain domain comprising three CDRs, wherein
(a) the VH CDR3 has an amino acid sequence selected from:
(i) VH CDR3 of monoclonal antibody 10E9.1 (SEQ ID NO.:49);
(ii) VH CDR3 of monoclonal antibody 15A1.2 (SEQ ID NO.:55); or
(iii) VH CDR3 of monoclonal antibody 15A11.1 (SEQ ID NO.:52), or
(b) the VL CDR3 has an amino acid sequence selected from:
(i) VL CDR3 of monoclonal antibody 10E9.1 (SEQ ID NO.:58);
(ii) VL CDR3 of monoclonal antibody 15A1.2 (SEQ ID NO.:64); or
(iii) VL CDR3 of monoclonal antibody 15A11.1 (SEQ ID NO.:61).

9. An antibody according to claim 8, wherein the antibody comprises a VH chain domain comprising three CDRs and a VL chain domain comprising three CDRs, wherein the three CDRs of the VH chain domain comprise
(a) (i) a VH CDR1 having the amino acid sequence of the VH CDR1 of monoclonal antibody 10E9.1 (SEQ ID NO.:47);
(ii) a VH CDR2 having the amino acid sequence of the VH CDR2 of monoclonal antibody 10E9.1 (SEQ ID NO.:48); and
(iii) a VH CDR3 having the amino acid sequence of the VH CDR3 of monoclonal antibody 10E9.1 (SEQ ID NO.:49), or
(b) (i) a VH CDR1 having the amino acid sequence of the VH CDR1 of monoclonal antibody 15A1.2 (SEQ ID NO.:53);
(ii) a VH CDR2 having the amino acid sequence of the VH CDR2 of monoclonal antibody 15A1.2 (SEQ ID NO.:54); and
(iii) a VH CDR3 having the amino acid sequence of the VH CDR3 of monoclonal antibody 15A1.2 (SEQ ID NO.:55), or
(c) (i) a VH CDR1 having the amino acid sequence of the VH CDR1 of monoclonal antibody 15A11.1 (SEQ ID NO.:50);
(ii) a VH CDR2 having the amino acid sequence of the VH CDR2 of monoclonal antibody 15A11.1 (SEQ ID NO.:51); and
(iii) a VH CDR3 having the amino acid sequence of the VH CDR3 of monoclonal antibody 15A11.1 (SEQ ID NO.:52).

10. An antibody according to claim 8, wherein the antibody comprises a VH chain domain comprising three CDRs and a VL chain domain comprising three CDRs, wherein the three CDRs of the VL chain domain comprise:
(a) (i) a VL CDR1 having the amino acid sequence of the VL CDR1 of monoclonal antibody 10E9.1 (SEQ ID NO.:56);
(ii) a VL CDR2 having the amino acid sequence of the VL CDR2 of monoclonal antibody 10E9.1 (SEQ ID NO.:57); and
(iii) a VL CDR3 having the amino acid sequence of the VL CDR3 of monoclonal antibody 10E9.1 (SEQ ID NO.:58), or
(b) (i) a VL CDR1 having the amino acid sequence of the VL CDR1 of monoclonal antibody 15A1.2(SEQ ID NO.:62);
(ii) a VL CDR3 having the amino acid sequence of the VL CDR2 of monoclonal antibody 15A1.2 (SEQ ID NO.:63); and
(iii) a VL CDR3 having the amino acid sequence of the VL CDR3 of monoclonal antibody 15A1.2 (SEQ ID NO.:64), or.
(c) (i) a VL CDR1 having the amino acid sequence of the VL CDR1 of monoclonal antibody 15A11.1 (SEQ ID NO.:59);
(ii) a VL CDR2 having the amino acid sequence of the VL CDR2 of monoclonal antibody 15A11.1 (SEQ ID NO.:60); and
(iii) a VL CDR3 having the amino acid sequence of the VL CDR3 of monoclonal antibody 15A11.1(SEQ ID NO.:61).

11-13. (canceled)

14. The antibody according to claim 1, wherein said antibody comprises a sequence comprising
a) SEQ ID NO.: 2 and SEQ ID NO.: 4;
b) SEQ ID NO.: 6 and SEQ ID NO.: 8; or
c) SEQ ID NO.: 10 and SEQ ID NO.: 12.

15. The antibody according to claim 1, wherein said antibody is any one of the monoclonal antibodies as shown in Table 1;
monoclonal antibody 10E9.1;
monoclonal antibody 15A1.2; or
monoclonal antibody 15A11.1.

16. An antibody which cross-competes with the antibody of claim 1 for binding to heparanase.

17. An antibody that binds to the same epitope on heparanase as the antibody of claim 1.

18. An antibody according to claim 1 wherein said antibody is
a monoclonal antibody;
a fully human monoclonal antibody; or
a binding fragment of a fully human monoclonal antibody.

19. (canceled)

20. A nucleic acid molecule encoding the antibody of claim 17.

21. A vector comprising the nucleic acid molecule of claim 20.

22. A host cell comprising the vector of claim 21.

23. A method of treating a proliferative, angiogenic, cell adhesion or invasion-related disease in an animal, comprising:
selecting an animal in need of treatment for a proliferative, angiogenic, cell adhesion or invasion-related disease; and
administering to said animal a therapeutically effective dose of the antibody of claim 1.

24. The method of claim 23, wherein said proliferative, angiogenic, cell adhesion or invasion-related diseases selected from the group consisting of: melanoma, skin cancer, small cell lung cancer, non-small cell lung cancer, salivary gland, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, thyroid cancer, endometrial cancer, kidney cancer, colon cancer, colorectal cancer, pancreatic cancer, esophageal carcinoma, brain/CNS cancers, neuronal cancers, head and neck cancers, mesothelioma, sarcomas, biliary (cholangiocarcinoma), small bowel adenocarcinoma, pediatric malignancies, epidermoid carcinoma, sarcomas, cancer of the pleural/peritoneal membranes and leukaemia, including acute myeloid leukaemia, acute lymphoblastic leukaemia, and multiple myeloma.

25. A method of treating a non-neoplastic disease in an animal, comprising:
selecting an animal in need of treatment for a non-neoplastic disease and
administering to said animal a therapeutically effective dose of the antibody of claim 1.

26. The method of claim 25, wherein said non-neoplastic disease is selected from the group consisting of arthritis, atherosclerosis and nephropathy.

27. (canceled)
